SEQUENCE LISTING (57 sequences)

SEQ ID NO: 1 - DNA, 15958 bp, Homo sapiens
  CDS (1)..(15147)
atgcccaagttcaagatgccattgttcggggcgtcagccccaggcaagtccatggaggcctcggtggatgtgtctgcgccgaaggtggaggccgacgtgagcctcctctccatgcagggggacctcaagaccactgacctcagcgtccagaccccttccgctgacctggaggtccaggatggccaagtggatgtgaaacttccggagggccccctgcccgagggagccagcctcaaagggcacctgcccaaggtgcagaggcccagtttgaagatgcccaaagtggacctcaagggccccaagctggacctgaaaggccccaaggcggaagtgacagcccccgatgtgaagatgtctctgtccagcatggaggtggacgtccaggccccgagagcaaagctggatggtgcgcggctggagggggacctgtccctggccgacaaggaggtgactgccaaagacagcaagttcaaaatgcccaagttcaagatgccatcattcggggtgtcggccccaggcaagtccatggaggactcggtggatgtgtctgcgccgaaggtggaggccgacgtgagcctctcctccatgcagggggacctcaaggccactgacctcagcattcagcccccttccgctgacctggaggtccaggctggccaagtggatgtgaaacttccggagggccctgtgcccgagggagccggccccaaagtgcacctgcccaaagtggagatgcccagtttcaagatgcccaaagtggacctcaagggcccccagatagatgttaagggccccaagctggacctgaaaggccccaaggcggaagtgacagcccccgatggcgaggtgtctctgcccagcatggaggtggatgtccaggcccagaaggccaagctggatggtgcgtggctggagggggacctgtccctggccgacaaggacgtgactgccaaagacagcaagttcaaaatgcccaagttcaagatgccgtcgttcggggtatcggccccagggaagtccatcaaggccttggtggatgtgtctgcacccaaggtggaggccgacctgagtctcccctccatgcagggggacctgaagaccactgacctcagcattcagcctgcttctactgacctgaaggtccaggctgaccaggtggatgtgaagctcccggagggccacctgcccgagggagctggccttaaagggcacttgcccaaggtggagatgcccagtttcaagatgcccaaagtggccctcaagggcccccaggtggacgtcaagggccccaagctggacctgaaaagccccaaggcggaagtcacagcccctgatgtggaggtgtctctgcccagcgtggaggtggacgtcgaggccccgggagccaagctggacagtgcgcggctggagggggaactgtccctggccgacaaggatgtgactgccaaagacagcaggttcaaaatgcccaagttcaagatgccatcgttcggggcgtcagccccaggcaagtccatcgaggcctcggtggatgtgtctgcacccaaagtggaggccgacgtgagtctcccctccatgcagggggacctcaagaccactgacctcagcattcagcccccttccgctgacctggaggtccacgctggccaggtggacgtgaagctcctggagggccacgtgcctgagggagccggcttcaaagggcacctgcccaaggtgcagatgcctagtttgaagatgcccaaagtggacctcaagggcccccaggtggaagtcaggggccccaagctggacctgaaaggtcataaggcagaggtgacggcccacgaagtggctgtgtctctgcccagtgtggaggtggacatgcaggccccgggagccaagttggatggcgcacagctggacggggacctgtccctggctgacaaggacgtgactgccaaagacagcaagttcaaaatgcccaagttcaagatgccgtcgttcggggtgtctgccccaggcaagtccattgaggcctccgtggacctgtctgcacccaaggtggaggccgacatgagcctcccctccatgcagggggacctcaagaccactgacctcagcattcagcccccttccactgacctggagctccaggctggccaattggacgtgaaactcccagagggccccgtgcccgagggagccggcctcaaagggcacctgcccaagctgcagatgcccagtttcaaggtgcccaaagtggacctcaagggccctgaaatagacatcaagggccccaagctggacctaaaagaccccaaggtggaagtgacagcccctgatgtggaggtttctctgcccagcgtggaggtggatgtcgaggccccaggagccaagctggatggtggacggctggaggaggacatgtccctggccgacaaggacttgactaccaaagacagcaagttcaaaatgcccaagttcaagatgccgtcgttcggggtgtctgccccaggcaagtccatcgaggcctcagtggatgtgtctgcgccgaaggtggaggccgacgtgagcctcccctccatgcagggggacctcaaggccactgacctgagcatacagcccccttctgctgacctggaggtccaggctggccaagtggacgtgaaactcccagagggccctgtgtccgagggagccggcctcaaagggcacctgcccaaagtgcagatgcccagtttcaagatgcccaaagtggacctcaaggggccccagatagatgttaagggccccaagctggacctgaaaggccccaaggtggaagtgacagcccccgatgtgaagatgtctctgtccagcatggaggtggacgtccaggccccgagagcaaagctggatggtgcgcagctggagggggacctgtccctggccgacaaggcggtgactgccaaagacagcaagttcaaaatgcccaagttcaagatgccatcatttggggtgtcggccccaggcaagtccatcgaggcctcggtggatgtgtctgagccgaaggtggaagctgatgtgagcctcccctccatgcagggggacctgaagaccactgacctcagcattcagtccccttccgccgacctggaggtccaggctggccaagtgaacgtgaaactcccggagggcccccttcccgagggagccggcttcaaagggcacctccccaaggtgcagatgcccagtttgaagatgcccaaagtggccctcaagggcccccagatggacgtcaagggccccaagctggacctgaaaggccccaaggcggaggtgatggcccccgacgtggaggtgtctctgcccagcgtggaggtggacgtcgaggctccaggagccaagctggacagtgtgcggctggagggtgacctgtccctggccgacaaggatgtgactgccaaagacagcaagttcaaaatgcccaagttcaagatgccgtcgttcggggtgtctgccccaggcaagtccatcgaggcctcggtggatgtgtctgcgccgaaggtggaggccgaagtgagcctcccctccatgcagggggacctcaagaccacggacctctgcattccgctcccttctgcagacctggtggtccaggctggccaagtggacatgaagctcccggagggccaggtgcccgagggagccggcctcaaagggcacttgcccaaggtggatatgcccagtttcaagatgcccaaagtggacctcaagggcccccagacagatgttaagggcgccaagctggacctgaaaggccccaaggcggaagtgacagcccccgatgtcgaggtgtctctgcccagcatggaggtggatgtccaggcccagaaggctaagctggatggtgcgcggctggagggagacctgtccctggccgacaaggacatgactgccaaagacagcaagttcaaaatgcccaaattcaagatgccgtcgttcggggtatcggccccagggaggtccatcgaggcctcggtggatgtgcctgcacccaaggtggaggccgacgtgagtctcccctccatgcagggggacctgaagaccactgacctcagcattcagcccccttctgccgacctgaaggtccagactggccaggtggatgtgaagctcccggagggccacgtgcccgagggagctggcctcaaagggcacctgcccaaggtggagatgcccagtttgaagatgcccaaagtggacctcaagggcccccaggtggacatcaagggccccaaactggacctaaaagaccccaaggtggaaatgagagtccccgatgtcgaggtgtctctgcccagcatggaggtggacgtccaggccccaagagccaagctggatagtgcgcatctgcagggggacctgaccctggccaacaaggacctgactaccaaagacagcaagttcaaaatgcccaagttcaagatgccgtcgtttggggtgtctgccccaggcaagtccatcgaggcctcggtggatgtgtctccacccaaggtggaggccgacatcaagggccccaagctggacctaaaagaccccaaggtggaagtgacagcccctgatgtggaggtgtctctgcccagcgtggaggtggacgtcaaggccccaggagccaagctggatggtgcgcggctggagggggacatgtccctggccgacaaggacgtgactgccaaagacagcaagttcaaaatgcccaagttcaagatgctgtcgtttggggtgtctgcccttggcaagtccatcgaggcctcagcggatgtgtctgcgttgaaggtggaggccgacgtgagcctcccctccatgcagggggacctcaagaccactgacctcagcgttcagcccccttccgctgacctggaggtccaggctggccaagtggatgtgaaactcccagagggccccgtgccggagggagccggcctcaaagggcacctgcccaagctgcagatgcccagtttcaagatgcccaaagtagatctcaagggcccccagatagatgtcaagggccccaagctggacctgaaaggccccaagacggacgtgatggcccccgacgtggaggtgtctcagcccagcgtggaggtggatgtcgaggccccgggagccaagctggatggtgcgtggctggagggggacctgtctgtggcggacaaggatgtgactaccaaagacagcaggttcaaaattcccaagttcaagatgccgtcattcggggtgtctgccccaggcaagtccatcgaggcctcggtggatgtgtctgcgccgaaggtggaggccgacgggagcctctcctccatgcagggggacctcaaggccactgacctcagcattcagcccccttccgctgacctggaggtccaggctggccaagtggacgtgaaactcccagagggccctgtgccggagggagccggcctcaaagggcacctgcccaaggtgcagatgcccagtttcaagatgcctgaaatggacctcaagggcccccagctagatgtcaagggccccaagctggacctgaaaggccccaaggcggaagtgacagcccccgatgtggagatgtctctgtccagcatggaggtggacgtccaggccccgagagcaaagctggatggtgcgcggctggagggggacctgtccctggccgacaagggtgtgacagccaaagatagcaagttcaaaatgcccaagttcaagatgccatcattcagggtgtcggccccaggcgagtccatcgaggcgttggtggatgtgtctgagctgaaggtggaagccgacatgagcctcccctccatgcaaggggaccttaagaccactgacatcagcattcagcccccctctgcccaactggaggtccaggctggccaggtggatgtgaaactcccagagggccacgttcccgagggagccggcctcaaagggcacctgcccaagctgcagatgcccagtttcaagatgcctgaagtggacctcaagggcccccagatagatgttaagggccccaacgtggacctgaaaggccccaaggcggaagtgacagcccccgatgtgaagatgtctctgtccagcatggaggtggacgtccaggccccgagagcaaagctggatggtgcgcggctggagggggacctgtccctggccgacaagggcatgacagccaaagacagcaagttcaaaatgcccaagttcaagatgccgtcattcggggtgtcggccccaggcaagtccatcgaggcctcggtggatgtgtctgagctgaaggtggaagctgacgggagcttcccctccatgcaaggggatcttaagaccactgacatccgcattcagcccccctccgcccaactggaggtccaggctggccaggtggacgtgaaactcccagagggccacgttcccgagggagccggcctcaaagggcacctgcccaaggtgcagatgcccagtttcaagatgcccaaagtggatctcaagggcccccagatagacgtcaagggccccaagctggacctgaaaggccccaaggcggaggtgacggcccccgacgtggaggtgtctctgcccagcgtggaggtggacgtcgaggccccgagagcaaagctggatggtgcacggctggagggtgacctgtccctggccgacaaggatgtgactgccaaagacagcaagttcaaaatgcccaagttcaagatgccgtcgttcggggtgtctgccccaggcaagtccattgaggtctcggtggatgtgtctgcgccgaaggtggaggccgaagtgagcctcccctccatgcagggggacctgaagaccactgacatcagcattgagcccccctctgcccaactggaggtccaggctggccaggtggacctgaagctcccagagggccacgttcccgagggagctggcctcaaagggcacctgcccaagttgcagatgcccagtttcaagatgcccaaagtagatcgcaagggaccccagatagatgtcaagggccccaagctggacctgaaaggcccgaagacggacgtgacggcccccgacgtggaggtgtctcagcccggcatggaggtggatgtcgaggccccaggagccaagttggatggtgcacggctggagggggacctgtccctggccgacaaggatgtgactgccaaagacagcaagttcaaaatgcccaagttcaagatgccgtcgttcggggtgtctgccccaggcaagtccattgaggtcttggtggatgtgtctgcgccaaaggtggaggccgacctgagcctcccctccatgcagggggacctgaagaacactgacatcagcattgagcccccctctgcccaactggaggtccaggctggccaggtggacgtgaagctcccagagggccacgttctcgagggagctggcctcaaagggcacctgcccaagttgcagatgcccagtttcaagatgcccaaagtagatcgcaagggcccccagatagacatcaagggccccaagctggacctgaaaggcccgaagatggatgtgacggcccccgacgtggaggtgtctcagcccagcatggaggtggacgtcgaggccccaggagccaagttggatggtgcacggctggagggggacctgtccctggccgacaaggatgtgactgccaaagacagcaagttcaaaatgcccaaattcaagatgccgtcgtacagggcgtctgccccaggcaagtccatccaggcctcggtggatgtgtctgcgccgaaggcggaggccgacgtgagcctcccctccatgcagggggacctcaagaccactgacctcagcattcagctcccttctgtggacctggaggtccaggctggccaggtggacgtgaagctcccggagggccacgtgcccgagggagctggcctcaaagggcacctgcccaaggtggagatgcccagtttcaagatgcccaaagtggacctcaagagcccccaggtggacatcaagggccccaagctggacctaaaagtccccaaggcggaagtgacagtccctgatgtggaggtgtctctgcccagcgtggaggtggacgtccaggccccgagagccaagctggatggtgcgcggctggagggggacctgtccctggctgaaaaggatgtgactgccaaagacagcaagttcaaaatgcccaagttcaagatgccctccttcggggtgtcggccccaggcaggtccatcgaggcctcgctggatgtgtctgcgccgaaggtggaggccgacgtgagcctctcctccatgcagggggacctcaaggccactgacctcagcattcagcccccttccgctgacctggaggtccaggctgtccaagtggatgtggaactcctggagggccccgtgcccgagggagccggcctcaaagggcacctgcccaaagtggagatgcccagtttaaagacgcccaaagtggacctcaagggcccccagatagatgttaagggccccaagctggacctgaaaggccccaaggcagaagtgagagtccccgatgtcgaggtgtctctgcccagcgtggaggtggatgtccaggccccgaaggccaagctggatgctgggcggctggagggagacctgtccctggctgacaaggacgtgactgccaaagacagcaagttcaaaatgcccaaattcaagatgccgtcattcagggtatcggccccagggaagtccatggaggcctcggtggatgtgtctgcacccaaggtggaagccgatgtgagtctcccctccatgcagggggacctgaagaccactgacctcagcattcagcccccttctgccgacctgaaggtccaggctggccagatggatgtgaagctcccggagggccaggtgcccgagggagccggcctcaaagagcacctgcccaaggtggagatgcccagtttgaagatgcccaaagtggacctcaagggcccccaggtggacatcaagggccccaagctggacctaaaagtctccaaggcggaagtcacagcccctgatgtggaggtgtctctgcccagcgtggaggtggacgtccaggccccaagagccaaactggatagtgcacagctggagggggacctgtccctggccgacaaggatgtgactgccaaagacagcaaattcaaaatgcccaagttcaagatgccgtcatttggggtgtctgccccaggcaagtccattgaggcctcggtgcacgtgtctgcacccaaggtggaggccgatgtgagtctcccctccatgcagggggacctcaagaccactgacctcagcattcagccccattctgccgacctgacggtccaagctcgccaggtggacatgaaactcctggagggccacgtgcccgaggaagccggcctcaaaggacacctgcccaaggtgcagatgcccagtttcaagatgcccaaagtcgacctcaagggccctgaaatagacatcaagggccccaagctggacctaaaagaccccaaggtggaagtgacagcccctgatgtggaggtttctctgcccagcgtggaggtggacgtcgaggccccaggagccaagctggatggtgcgcggctggagggggacctgtccctggccgacaaggacatgacggccaaagacagcaagttcaaaatgcccaagttcaagatgccgtcgttcggggtgtctgccccaggcaagtccatggaggcatcagtggatgtgaccgcgccaaaggtggaggccgacgtgagcctcccttccatgcagggggacctcaaggccactgacctcagcgttcagcccccttccgctgacctggaggtccaggctggccaagtggacgtgaaactcccagagggccccgtgcccgagggagccagcctcaaagggcacctgcccaaggtgcagatgcccagtttcaagatgcccaaagtggacctcaagggcccccagatagatgttaagggccccaagctggacctgaaaggccccaaggcggaagtgacagcccctgatgtgaagatgtctctgtccagcatggaggtggacgtccaggccccgagagcaaagctggatggtgtgcagctggagggggacctgtccctggccgacaaggatgtgactgccaaagacagcaagttcaaaatgcccaagttcaagatgccatcattcggggtgtcggccccaggcaagtccatggaggcgtccgtggatgtgtctgagctgaaggcgaaagccgacgtgagcctcccctccatgcagggggacctcaagaccactgacctcagcattcagtccccttccgccgacctggaggtccaggctggccaagtggacgtgaaactcccggagggccccctgcccaagggagccggcctcaaagggcacctccccaaggtgcagatgccctgtttgaagatgcccaaagtggccctcaagggcccccaggtggatgtcaagggccccaagctggacctgaaaggccccaaggcggatgtgatgacccccgtcgtggaggtgtctctgcccagcatggaggtggacgtcgaggccccgggagccaagctggacagtgtgcggctggagggtgacctgtccctagccgacaaggacatgactgccaaagacagcaagttcaaaatgcccaagttcaagatgccgtcgttcggggtgtctgccccaggcaagtccatcgaggcctcgttggatgtgtctgcgctgaaggtggaggctgacgtgagcctcccctccatgcagggggacctgaagaccactcacctcagcattcagcccccttccgctgatctggaggtccaggctggccaagaggatgtgaaactcccagagggccctgtgcatgagggagccggcctcaaagggcacctgccgaagctgcagatgcccagtttcaaggtacccaaagtggacctcaagggtccccagatagacgttaatgtccccaagctggacctgaaaggccccaaggtggaggtgacgtcccccaacctggacgtgtctctgcccagcatggaggtggacatccaagccccaggagccaagctggacagtacgcggctggagggggacctgtccctggctgacaaggacgtgactgccaaagacagcaagttcaaaatgcccaagttcaagatgccatcctttgggatgttgtccccaggcaagtccatcgaggtctcggtggatgtgtctgcgccaaagatggaggccgacatgagcattccctccatgcagggggacctcaagaccactgacctccgcattcaggccccttccgccgacctggaggtccaggctggccaggtggacttgaaacttccagaaggccacatgcccgaggtagccggcctcaaagggcacctgcccaaggtggagatgcccagtttcaagatgcccaaagtggacctcaagggcccccaggtggacgtcaagggccccaagctggacctgaaaggcccaaaggcagaggtgatggcccccgatgtggaggtgtctctgcccagcgtggagacggatgtccaggccccaggatccatgctggatggtgcgcggcttgagggggacctgtccctggcccacgaggatgtagctgggaaagacagtaagtttcaaggaccaaaactgagcacgtctggttttgaatggtcgtcaaagaaagtttccatgtcttcctctgaaatcgaaggaaatgttacattccatgagaagacttccacatttcccattgtggaatctgttgttcatgaaggtgatcttcatgatccatctcgcgatggtaacttggggcttgctgttggagaagttggaatggattcgaagtttaagaaactgcattttaaagtgcccaaagtttcattttcttctaccaaaactcctaaagatagtttagtcccaggtgcaaagtctagcataggtctttccacgattcctttatcatcttcagaatgctcaagttttgaattacaacaggtttcggcttgttcagagccatccatgcagatgcctaaggtgggttttgctgggtttccatcatcccggcttgatctcactggtcctcactttgaatcttctattctctctccctgtgaggatgttacacttacaaaataccaggtgactgttcccagagctgccttggcccctgagcttgctctggaaattccttctgggtctcaggctgatattcctcttcccaagacagagtgctccactgacctgcagcctccagagggagttccaacatctcaagctgagagtcactctggcccactgaattccatgattcctgtttctcttggtcaggtatcttttcctaaattctataaaccaaagtttgtgttttcagtcccccaaatggcagttcctgagggagacctacatgcagcagtgggtgccccagtcatgtctcctcttagccctggagaaagagtgcagtgccccttgccaagcacccagctgccatccccaggcacctgtgtgtctcagggcccagaagagcttgtggcctccttgcagacatcagtagtggcccctggagaagccccttctgaagatgctgaccacgaagggaaagggagtcccttgaaaatgcctaagattaagcttccatcatttaggtggtccccgaagaaggaaacagggccaaaggtggacccagaatgcagcgtggaggactcaaaactcagcctggttttagacaaggatgaagtggccccgcagtctgccatccacatggatctgcctcctgagagggatggagagaaggggaggagcacaaagcctggctttgccatgccaaaacttgcacttcccaaaatgaaggcttctaagagtggggtcagcctgccacagagagacgtggatccttccctttctagtgccacagcagggggtagctttcaagacacagaaaaggccagcagtgacggtggtaggggaggacttggtgcaacagcaagtgccacaggaagtgagggtgtgaacctccaccggccacaggtccacattcccagtttgggctttgccaaacctgatctcagatcctccaaggccaaggtggaggtgagccagcctgaagctgacctgcctcttcccaaacatgatctgtctaccgaaggtgacagcagaggatgtgggctcggggatgtcccagtgagccagccttgtggggaggggatagcccccacacctgaagatcccctccagccatcctgtagaaaaccagatgctgaagtcctcacagtggaaagcccagaggaggaagccatgaccaagtactcgcaggaaagctggtttaaaatgcccaagttccgcatgcccagccttaggcgctctttcagggacagaggcggggctggaaagctggaagtggctcagacacaggcaccggcagcaacagggggtgaagcagcagctaaagtcaaagagttccttgtttctgggtcaaacgtggaggcagctatgtccctacagctcccagaggcagatgcagaagtgacagcttctgagagcaaatcatccacagatattctaaggtgtgatcttgacagcacaggcttgaagctgcacctctccactgctgggatgactggggatgagctttccacttctgaggtcaggatccatccatccaaaggacctctcccttttcagatgcctggcatgaggcttccagaaacccaggttcttccaggagaaatagatgagactcctctttccaagccaggacatgaccttgccagcatggaggataaaacagagaaatggtcttcccagcctgaaggtccacttaaattgaaagcttcaagtactgatatgccatcccagatttctgtggttaatgtggatcaactgtgggaagattctgtcctaactgtcaaattccccaaattaatggtaccaaggttctccttccctgcccccagctcagaggatgatgtgttcatccccactgtgagggaagtgcagtgtccagaggccaatattgatacagccctttgtaaggaaagtccggggctctggggagccagcatcctgaaggcaggtgctggggtccctggggagcagcctgtggaccttaacctgcctttggaagctcccccaatttcaaaggtcagagtgcatattcagggtgctcaggttgaaagtcaagaggtcactatacacagcatagtgacaccagagtttgtagatctctcagtacccaggactttttccactcagattgtgcgggaatcagagatccccacgtcagagattcaaacaccttcgtacggattttccttattaaaagtgaaaatcccagagccccacacgcaggctagagtgtacacaacaatgactcaacactctaggactcaggagggcacagaagaggctcccatacaagccaccccaggagtagactccatttctggagatctccagcctgacactggagaaccatttgagatgatctcttccagcgtcaatgtactgggacagcaaacactcacatttgaagttccttctggccaccagcttgcagacagctgttcagatgaggagccagcagaaattcttgagtttccccctgatgatagccaagaggcaaccacaccactggcagatgaaggcagggctccaaaagacaaaccagaaagtaaaaaatctggtctgctctggttttggcttccaaacattgggttttcctcttctgttgatgagacaggtgttgattccaaaaatgacgtccagagatctgctcccattcaaacacagcctgaggcacgaccagaggcagaactgcctaaaaaacaggagaaggcaggctggttccgatttcccaaattagggttctcctcatctcctaccaagaaaagcaaaagcaccgaagatggggcagagctggaagaacaaaaacttcaagaagaaacaatcacgttttttgatgcccgagaaagtttctcccctgaagagaaggaagagggtgaactgatcgggcctgtgggcactgggctggactccagagtgatggtgacatccgcggcaagaacagagttaatcctgcccgagcaggacagaaaagctgacgatgaaagcaaagggtcaggcctgggaccaaatgaaggctgagaggtatggctcatcagtacaagagagatgcaaaaaactaagttggaaagtaaaggctacacacacatatggagcaccccatcccacagcacattacatccacctcacttcacagaacggagaacagagcagaaatgaccagaacacctttgtcaccatcacacagccctcctaaaatggaaccaaagcttcccagctccctcaaagctttggatgcaaagaaggcaccctgacttccacaagacaccagaattcacacggtactcagaggcactgctggggaagtttgttggtctttattagataaatttccagagacctgtccataatacccaacagaacatgactgtttctttgaggaaagggttataatgtctgtggtgtacaagtcgtttttggtataacttctttcctgctgctgctgcttcccggcaaacatagttttcctatttcaggcagagtgcggtatattccaggaaacactgtttcctactcacttagcttacttctttgttgaatgcctcactaatggcaagtttcaagatgttttgggtgacaatgcacacatgctgggcaaaagggtgatggccagtggctggcagctgggccagcagaagctaggacatctgtgagttgtcattctcatctatccatgtccactggcctgccagcatccgccagtgccttgccagtgtgcacggtcccacactgtggcccctgagtcccctaatgtacacgctgcagccagaatgcagatggagctggcttggctgttccctggatgggcaataaagaaagtgctgcatcccat

SEQ ID NO: 2 - protein, 5048 aa, Homo sapiens
MPKFKMPLFGASAPGKSMEASVDVSAPKVEADVSLLSMQGDLKTTDLSVQTPSADLEVQDGQVDVKLPEGPLPEGASLKGHLPKVQRPSLKMPKVDLKGPKLDLKGPKAEVTAPDVKMSLSSMEVDVQAPRAKLDGARLEGDLSLADKEVTAKDSKFKMPKFKMPSFGVSAPGKSMEDSVDVSAPKVEADVSLSSMQGDLKATDLSIQPPSADLEVQAGQVDVKLPEGPVPEGAGPKVHLPKVEMPSFKMPKVDLKGPQIDVKGPKLDLKGPKAEVTAPDGEVSLPSMEVDVQAQKAKLDGAWLEGDLSLADKDVTAKDSKFKMPKFKMPSFGVSAPGKSIKALVDVSAPKVEADLSLPSMQGDLKTTDLSIQPASTDLKVQADQVDVKLPEGHLPEGAGLKGHLPKVEMPSFKMPKVALKGPQVDVKGPKLDLKSPKAEVTAPDVEVSLPSVEVDVEAPGAKLDSARLEGELSLADKDVTAKDSRFKMPKFKMPSFGASAPGKSIEASVDVSAPKVEADVSLPSMQGDLKTTDLSIQPPSADLEVHAGQVDVKLLEGHVPEGAGFKGHLPKVQMPSLKMPKVDLKGPQVEVRGPKLDLKGHKAEVTAHEVAVSLPSVEVDMQAPGAKLDGAQLDGDLSLADKDVTAKDSKFKMPKFKMPSFGVSAPGKSIEASVDLSAPKVEADMSLPSMQGDLKTTDLSIQPPSTDLELQAGQLDVKLPEGPVPEGAGLKGHLPKLQMPSFKVPKVDLKGPEIDIKGPKLDLKDPKVEVTAPDVEVSLPSVEVDVEAPGAKLDGGRLEEDMSLADKDLTTKDSKFKMPKFKMPSFGVSAPGKSIEASVDVSAPKVEADVSLPSMQGDLKATDLSIQPPSADLEVQAGQVDVKLPEGPVSEGAGLKGHLPKVQMPSFKMPKVDLKGPQIDVKGPKLDLKGPKVEVTAPDVKMSLSSMEVDVQAPRAKLDGAQLEGDLSLADKAVTAKDSKFKMPKFKMPSFGVSAPGKSIEASVDVSEPKVEADVSLPSMQGDLKTTDLSIQSPSADLEVQAGQVNVKLPEGPLPEGAGFKGHLPKVQMPSLKMPKVALKGPQMDVKGPKLDLKGPKAEVMAPDVEVSLPSVEVDVEAPGAKLDSVRLEGDLSLADKDVTAKDSKFKMPKFKMPSFGVSAPGKSIEASVDVSAPKVEAEVSLPSMQGDLKTTDLCIPLPSADLVVQAGQVDMKLPEGQVPEGAGLKGHLPKVDMPSFKMPKVDLKGPQTDVKGAKLDLKGPKAEVTAPDVEVSLPSMEVDVQAQKAKLDGARLEGDLSLADKDMTAKDSKFKMPKFKMPSFGVSAPGRSIEASVDVPAPKVEADVSLPSMQGDLKTTDLSIQPPSADLKVQTGQVDVKLPEGHVPEGAGLKGHLPKVEMPSLKMPKVDLKGPQVDIKGPKLDLKDPKVEMRVPDVEVSLPSMEVDVQAPRAKLDSAHLQGDLTLANKDLTTKDSKFKMPKFKMPSFGVSAPGKSIEASVDVSPPKVEADIKGPKLDLKDPKVEVTAPDVEVSLPSVEVDVKAPGAKLDGARLEGDMSLADKDVTAKDSKFKMPKFKMLSFGVSALGKSIEASADVSALKVEADVSLPSMQGDLKTTDLSVQPPSADLEVQAGQVDVKLPEGPVPEGAGLKGHLPKLQMPSFKMPKVDLKGPQIDVKGPKLDLKGPKTDVMAPDVEVSQPSVEVDVEAPGAKLDGAWLEGDLSVADKDVTTKDSRFKIPKFKMPSFGVSAPGKSIEASVDVSAPKVEADGSLSSMQGDLKATDLSIQPPSADLEVQAGQVDVKLPEGPVPEGAGLKGHLPKVQMPSFKMPEMDLKGPQLDVKGPKLDLKGPKAEVTAPDVEMSLSSMEVDVQAPRAKLDGARLEGDLSLADKGVTAKDSKFKMPKFKMPSFRVSAPGESIEALVDVSELKVEADMSLPSMQGDLKTTDISIQPPSAQLEVQAGQVDVKLPEGHVPEGAGLKGHLPKLQMPSFKMPEVDLKGPQIDVKGPNVDLKGPKAEVTAPDVKMSLSSMEVDVQAPRAKLDGARLEGDLSLADKGMTAKDSKFKMPKFKMPSFGVSAPGKSIEASVDVSELKVEADGSFPSMQGDLKTTDIRIQPPSAQLEVQAGQVDVKLPEGHVPEGAGLKGHLPKVQMPSFKMPKVDLKGPQIDVKGPKLDLKGPKAEVTAPDVEVSLPSVEVDVEAPRAKLDGARLEGDLSLADKDVTAKDSKFKMPKFKMPSFGVSAPGKSIEVSVDVSAPKVEAEVSLPSMQGDLKTTDISIEPPSAQLEVQAGQVDLKLPEGHVPEGAGLKGHLPKLQMPSFKMPKVDRKGPQIDVKGPKLDLKGPKTDVTAPDVEVSQPGMEVDVEAPGAKLDGARLEGDLSLADKDVTAKDSKFKMPKFKMPSFGVSAPGKSIEVLVDVSAPKVEADLSLPSMQGDLKNTDISIEPPSAQLEVQAGQVDVKLPEGHVLEGAGLKGHLPKLQMPSFKMPKVDRKGPQIDIKGPKLDLKGPKMDVTAPDVEVSQPSMEVDVEAPGAKLDGARLEGDLSLADKDVTAKDSKFKMPKFKMPSYRASAPGKSIQASVDVSAPKAEADVSLPSMQGDLKTTDLSIQLPSVDLEVQAGQVDVKLPEGHVPEGAGLKGHLPKVEMPSFKMPKVDLKSPQVDIKGPKLDLKVPKAEVTVPDVEVSLPSVEVDVQAPRAKLDGARLEGDLSLAEKDVTAKDSKFKMPKFKMPSFGVSAPGRSIEASLDVSAPKVEADVSLSSMQGDLKATDLSIQPPSADLEVQAVQVDVELLEGPVPEGAGLKGHLPKVEMPSLKTPKVDLKGPQIDVKGPKLDLKGPKAEVRVPDVEVSLPSVEVDVQAPKAKLDAGRLEGDLSLADKDVTAKDSKFKMPKFKMPSFRVSAPGKSMEASVDVSAPKVEADVSLPSMQGDLKTTDLSIQPPSADLKVQAGQMDVKLPEGQVPEGAGLKEHLPKVEMPSLKMPKVDLKGPQVDIKGPKLDLKVSKAEVTAPDVEVSLPSVEVDVQAPRAKLDSAQLEGDLSLADKDVTAKDSKFKMPKFKMPSFGVSAPGKSIEASVHVSAPKVEADVSLPSMQGDLKTTDLSIQPHSADLTVQARQVDMKLLEGHVPEEAGLKGHLPKVQMPSFKMPKVDLKGPEIDIKGPKLDLKDPKVEVTAPDVEVSLPSVEVDVEAPGAKLDGARLEGDLSLADKDMTAKDSKFKMPKFKMPSFGVSAPGKSMEASVDVTAPKVEADVSLPSMQGDLKATDLSVQPPSADLEVQAGQVDVKLPEGPVPEGASLKGHLPKVQMPSFKMPKVDLKGPQIDVKGPKLDLKGPKAEVTAPDVKMSLSSMEVDVQAPRAKLDGVQLEGDLSLADKDVTAKDSKFKMPKFKMPSFGVSAPGKSMEASVDVSELKAKADVSLPSMQGDLKTTDLSIQSPSADLEVQAGQVDVKLPEGPLPKGAGLKGHLPKVQMPCLKMPKVALKGPQVDVKGPKLDLKGPKADVMTPVVEVSLPSMEVDVEAPGAKLDSVRLEGDLSLADKDMTAKDSKFKMPKFKMPSFGVSAPGKSIEASLDVSALKVEADVSLPSMQGDLKTTHLSIQPPSADLEVQAGQEDVKLPEGPVHEGAGLKGHLPKLQMPSFKVPKVDLKGPQIDVNVPKLDLKGPKVEVTSPNLDVSLPSMEVDIQAPGAKLDSTRLEGDLSLADKDVTAKDSKFKMPKFKMPSFGMLSPGKSIEVSVDVSAPKMEADMSIPSMQGDLKTTDLRIQAPSADLEVQAGQVDLKLPEGHMPEVAGLKGHLPKVEMPSFKMPKVDLKGPQVDVKGPKLDLKGPKAEVMAPDVEVSLPSVETDVQAPGSMLDGARLEGDLSLAHEDVAGKDSKFQGPKLSTSGFEWSSKKVSMSSSEIEGNVTFHEKTSTFPIVESVVHEGDLHDPSRDGNLGLAVGEVGMDSKFKKLHFKVPKVSFSSTKTPKDSLVPGAKSSIGLSTIPLSSSECSSFELQQVSACSEPSMQMPKVGFAGFPSSRLDLTGPHFESSILSPCEDVTLTKYQVTVPRAALAPELALEIPSGSQADIPLPKTECSTDLQPPEGVPTSQAESHSGPLNSMIPVSLGQVSFPKFYKPKFVFSVPQMAVPEGDLHAAVGAPVMSPLSPGERVQCPLPSTQLPSPGTCVSQGPEELVASLQTSVVAPGEAPSEDADHEGKGSPLKMPKIKLPSFRWSPKKETGPKVDPECSVEDSKLSLVLDKDEVAPQSAIHMDLPPERDGEKGRSTKPGFAMPKLALPKMKASKSGVSLPQRDVDPSLSSATAGGSFQDTEKASSDGGRGGLGATASATGSEGVNLHRPQVHIPSLGFAKPDLRSSKAKVEVSQPEADLPLPKHDLSTEGDSRGCGLGDVPVSQPCGEGIAPTPEDPLQPSCRKPDAEVLTVESPEEEAMTKYSQESWFKMPKFRMPSLRRSFRDRGGAGKLEVAQTQAPAATGGEAAAKVKEFLVSGSNVEAAMSLQLPEADAEVTASESKSSTDILRCDLDSTGLKLHLSTAGMTGDELSTSEVRIHPSKGPLPFQMPGMRLPETQVLPGEIDETPLSKPGHDLASMEDKTEKWSSQPEGPLKLKASSTDMPSQISVVNVDQLWEDSVLTVKFPKLMVPRFSFPAPSSEDDVFIPTVREVQCPEANIDTALCKESPGLWGASILKAGAGVPGEQPVDLNLPLEAPPISKVRVHIQGAQVESQEVTIHSIVTPEFVDLSVPRTFSTQIVRESEIPTSEIQTPSYGFSLLKVKIPEPHTQARVYTTMTQHSRTQEGTEEAPIQATPGVDSISGDLQPDTGEPFEMISSSVNVLGQQTLTFEVPSGHQLADSCSDEEPAEILEFPPDDSQEATTPLADEGRAPKDKPESKKSGLLWFWLPNIGFSSSVDETGVDSKNDVQRSAPIQTQPEARPEAELPKKQEKAGWFRFPKLGFSSSPTKKSKSTEDGAELEEQKLQEETITFFDARESFSPEEKEEGELIGPVGTGLDSRVMVTSAARTELILPEQDRKADDESKGSGLGPNEG

SEQ ID NO: 3 - DNA, 2731 bp, Homo sapiens
  CDS (216)..(2318)
gcgcttggcgggagatagaaaagtgcttcaacccgcgccggcggcgactgcagttcctgcgagcgaggagcgcgggacctgctgacacgctgacgccttcgagcgcggcccggggcccggagcggccggagcagcccgggtcctgaccccggcccggctcccgctccgggctctgccggcgggcgggcgagcgcggcgcggtccgggccggggggatgtctcggcggacgcgctgcgaggatctggatgagctgcactaccaggacacagattcagatgtgccggagcagagggatagcaagtgcaaggtcaaatggacccatgaggaggacgagcagctgagggccctggtgaggcagtttggacagcaggactggaagttcctggccagccacttccctaaccgcactgaccagcaatgccagtacaggtggctgagagttttgaatccagaccttgtcaaggggccatggaccaaagaggaagaccaaaaagtcatcgagctggttaagaagtatggcacaaagcagtggacactgattgccaagcacctgaagggccggctggggaagcagtgccgtgaacgctggcacaaccacctcaaccctgaggtgaagaagtcttgctggaccgaggaggaggaccgcatcatctgcgaggcccacaaggtgctgggcaaccgctgggccgagatcgccaagatgttgccagggaggacagacaatgctgtgaagaatcactggaactctaccatcaaaaggaaggtggacacaggaggcttcttgagcgagtccaaagactgcaagcccccagtgtacttgctgctggagctcgaggacaaggacggcctccagagtgcccagcccacggaaggccagggaagtcttctgaccaactggccctccgtccctcctaccataaaggaggaggaaaacagtgaggaggaacttgcagcagccaccacatcgaaggaacaggagcccatcggtacagatctggacgcagtgcgaacaccagagcccttggaggaattcccgaagcgtgaggaccaggaaggctccccaccagaaacgagcctgccttacaagtgggtggtggaggcagctaacctcctcatccccgctgtgggttctagcctctctgaagccctggacttgatcgagtcggaccctgatgcttggtgtgacctgagtaaatttgacctccctgaggaaccatctgcagaggacagtatcaacaacagcctagtgcagctgcaagcgtcacatcagcagcaagtcctgccaccccgccagccttccgccctggtgcccagtgtgaccgagtaccgcctggatggccacaccatctcagacctgagccggagcagccggggcgagctgatccccatctcccccagcactgaagtcgggggctctggcattggcacaccgccctctgtgctcaagcggcagaggaagaggcgtgtggctctgtcccctgtcactgagaatagcaccagtctgtccttcctggattcctgtaacagcctcacgcccaagagcacacctgttaagaccctgcccttctcgccctcccagtttctgaacttctggaacaaacaggacacattggagctggagagcccctcgctgacatccaccccagtgtgcagccagaaggtggtggtcaccacaccactgcaccgggacaagacacccctgcaccagaaacatgctgcgtttgtaaccccagatcagaagtactccatggacaacactccccacacgccaaccccgttcaagaacgccctggagaagtacggacccctgaagcccctgccacagaccccgcacctggaggaggacttgaaggaggtgctgcgttctgaggctggcatcgaactcatcatcgaggacgacatcaggcccgagaagcagaagaggaagcctgggctgcggcggagccccatcaagaaagtccggaagtctctggctcttgacattgtggatgaggatgtgaagctgatgatgtccacactgcccaagtctctatccttgccgacaactgccccttcaaactcttccagcctcaccctgtcaggtatcaaagaagacaacagcttgctcaaccagggcttcttgcaggccaagcccgagaaggcagcagtggcccagaagccccgaagccacttcacgacacctgcccctatgtccagtgcctggaagacggtggcctgcggggggaccagggaccagcttttcatgcaggagaaagcccggcagctcctgggccgcctgaagcccagccacacatctcggaccctcatcttgtcctgaggtgttgagggtgtcacgagcccattctcatgtttacaggggttgtgggggcagagggggtctgtgaatctgagagtcattcaggtgacctcctgcagggagccttctgccaccagcccctccccagactctcaggtggaggcaacagggccatgtgctgccctgttgccgagcccagctgtgggcggctcctggtgctaacaacaaagttccacttccaggtctgcctggttccctccccaaggccacagggagctccgtcagcttctcccaagcccacgtcaggcctggcctcatctcagaccctgcttaggatgggggatgtggccaggggtgctcctgtgctcaccctctcttggtgcatttttttggaagaataaaattgcctctctcttaaaaaaaaaaaaaaaaaa

SEQ ID NO: 4 - protein, 700 aa, Homo sapiens
MSRRTRCEDLDELHYQDTDSDVPEQRDSKCKVKWTHEEDEQLRALVRQFGQQDWKFLASHFPNRTDQQCQYRWLRVLNPDLVKGPWTKEEDQKVIELVKKYGTKQWTLIAKHLKGRLGKQCRERWHNHLNPEVKKSCWTEEEDRIICEAHKVLGNRWAEIAKMLPGRTDNAVKNHWNSTIKRKVDTGGFLSESKDCKPPVYLLLELEDKDGLQSAQPTEGQGSLLTNWPSVPPTIKEEENSEEELAAATTSKEQEPIGTDLDAVRTPEPLEEFPKREDQEGSPPETSLPYKWVVEAANLLIPAVGSSLSEALDLIESDPDAWCDLSKFDLPEEPSAEDSINNSLVQLQASHQQQVLPPRQPSALVPSVTEYRLDGHTISDLSRSSRGELIPISPSTEVGGSGIGTPPSVLKRQRKRRVALSPVTENSTSLSFLDSCNSLTPKSTPVKTLPFSPSQFLNFWNKQDTLELESPSLTSTPVCSQKVVVTTPLHRDKTPLHQKHAAFVTPDQKYSMDNTPHTPTPFKNALEKYGPLKPLPQTPHLEEDLKEVLRSEAGIELIIEDDIRPEKQKRKPGLRRSPIKKVRKSLALDIVDEDVKLMMSTLPKSLSLPTTAPSNSSSLTLSGIKEDNSLLNQGFLQAKPEKAAVAQKPRSHFTTPAPMSSAWKTVACGGTRDQLFMQEKARQLLGRLKPSHTSRTLILS

SEQ ID NO: 5 - DNA, 1207 bp, Homo sapiens
  CDS (369)..(1037)
cctctccgccacttccctcgcttctgaccatagtttgcggggaagggagcgagcgcgtcgaaaaccaaggaacgtgcgcgctgacgtcacggttgaggctcggagctgaggggccgcggagggcgtggcctgcgggcggttataaagaggcagtggtgcgcgcgcggccggctcagtgctgccgggcaccggggcggcgggttggtctacgctgtgcgcggcggacgtcggaggcagcggggagcggagcggggccgccggggcctctccagggccgcagcggcagcagttgggccccccgccccggccggcggaccgaagaacgcaggaagggggccggggggacccgcccccggccggccgcagccatgaactccaacgtggagaacctacccccgcacatcatccgcctggtgtacaaggaggtgacgacactgaccgcagacccacccgatggcatcaaggtctttcccaacgaggaggacctcaccgacctccaggtcaccatcgagggccctgaggggaccccatatgctggaggtctgttccgcatgaaactcctgctggggaaggacttccctgcctccccacccaagggctacttcctgaccaagatcttccacccgaacgtgggcgccaatggcgagatctgcgtcaacgtgctcaagagggactggacggctgagctgggcatccgacacgtactgctgaccatcaagtgcctgctgatccaccctaaccccgagtctgcactcaacgaggaggcgggccgcctgctcttggagaactacgaggagtatgcggctcgggcccgtctgctcacagagatccacgggggcgccggcgggcccagcggcagggccgaagccggtcgggccctggccagtggcactgaagcttcctccaccgaccctggggccccagggggcccgggaggggctgagggtcccatggccaagaagcatgctggcgagcgcgataagaagctggcggccaagaaaaagacggacaagaagcgggcgctgcggcggctgtagtgggctctcttcctccttccaccgtgaccccaacctctcctgtcccctccctccaactctgtctctaagttatttaaattatggctggggtcggggagggtacagggggcactgggacctggatttgtttttctaaataaagttggaaaagcagaaaaaaaaaaaaaaaa

SEQ ID NO: 6 - protein, 222 aa, Homo sapiens
MNSNVENLPPHIIRLVYKEVTTLTADPPDGIKVFPNEEDLTDLQVTIEGPEGTPYAGGLFRMKLLLGKDFPASPPKGYFLTKIFHPNVGANGEICVNVLKRDWTAELGIRHVLLTIKCLLIHPNPESALNEEAGRLLLENYEEYAARARLLTEIHGGAGGPSGRAEAGRALASGTEASSTDPGAPGGPGGAEGPMAKKHAGERDKKLAAKKKTDKKRALRRL

SEQ ID NO: 7 - DNA, 927 bp, Homo sapiens
  CDS (126)..(719)
cgcgcagcgctggtaccccgttggtccgcgcgttgctgcgttgtgaggggtgtcagctcagtgcatcccaggcagctcttagtgtggagcagtgaactgtgtgtggttccttctacttggggatcatgcagagagcttcacgtctgaagagagagctgcacatgttagccacagagccacccccaggcatcacatgttggcaagataaagaccaaatggatgacctgcgagctcaaatattaggtggagccaacacaccttatgagaaaggtgtttttaagctagaagttatcattcctgagaggtacccatttgaacctcctcagatccgatttctcactccaatttatcatccaaacattgattctgctggaaggatttgtctggatgttctcaaattgccaccaaaaggtgcttggagaccatccctcaacatcgcaactgtgttgacctctattcagctgctcatgtcagaacccaaccctgatgacccgctcatggctgacatatcctcagaatttaaatataataagccagccttcctcaagaatgccagacagtggacagagaagcatgcaagacagaaacaaaaggctgatgaggaagagatgcttgataatctaccagaggctggtgactccagagtacacaactcaacacagaaaaggaaggccagtcagctagtaggcatagaaaagaaatttcatcctgatgtttaggggacttgtcctggttcatcttagttaatgtgttctttgccaaggtgatctaagttgcctaccttgaatttttttttaaatatatttgatgacataatttttgtgtagtttatttatcttgtacatatgtattttgaaatcttttaaacctgaaaaataaatagtcatttaatgttgaaaaaaaaaaaaaaaaaaaaaaaaaaaaaaa

SEQ ID NO: 8 - protein, 197 aa, Homo sapiens
MQRASRLKRELHMLATEPPPGITCWQDKDQMDDLRAQILGGANTPYEKGVFKLEVIIPERYPFEPPQIRFLTPIYHPNIDSAGRICLDVLKLPPKGAWRPSLNIATVLTSIQLLMSEPNPDDPLMADISSEFKYNKPAFLKNARQWTEKHARQKQKADEEEMLDNLPEAGDSRVHNSTQKRKASQLVGIEKKFHPDV

SEQ ID NO: 9 - DNA, 23 bp, Artificial
  An artificially synthesized primer sequence for RT-PCR
gagaaggaagagggtgaactgat

SEQ ID NO: 10 - DNA, 23 bp, Artificial
  An artificially synthesized primer sequence for RT-PCR
cagtggacatggatagatgagaa

SEQ ID NO: 11 - DNA, 20 bp, Artificial
  An artificially synthesized primer sequence for RT-PCR
gaagccacttcacgacacct

SEQ ID NO: 12 - DNA, 22 bp, Artificial
  An artificially synthesized primer sequence for RT-PCR
atcctaagcagggtctgagatg

SEQ ID NO: 13 - DNA, 23 bp, Artificial
  An artificially synthesized primer sequence for RT-PCR
tacttcctgaccaagatcttcca

SEQ ID NO: 14 - DNA, 23 bp, Artificial
  An artificially synthesized primer sequence for RT-PCR
ttagagacagagttggagggagg

SEQ ID NO: 15 - DNA, 23 bp, Artificial
  An artificially synthesized primer for RT-PCR
caaatattaggtggagccaacac

SEQ ID NO: 16 - DNA, 23 bp, Artificial
  An artificially synthesized primer for RT-PCR
tagatcaccttggcaaagaacac

SEQ ID NO: 17 - DNA, 20 bp, Artificial
  An artificially synthesized primer sequence for RT-PCR
aggatgcagaaggagatcac

SEQ ID NO: 18 - DNA, 20 bp, Artificial
  An artificially synthesized primer sequence for RT-PCR
agaaagggtgtaacgcaact

SEQ ID NO: 19 - DNA, 21 bp, Artificial
  An artificially synthesized primer sequence for RT-PCR
cacccccactgaaaaagatga

SEQ ID NO: 20 - DNA, 19 bp, Artificial
  An artificially synthesized primer sequence for RT-PCR
tacctgtggagcaacctgc

SEQ ID NO: 21 - DNA, 23 bp, Artificial
  An artificially synthesized primer sequence for RT-PCR
aaggattatgaggaggttggtgt

SEQ ID NO: 22 - DNA, 23 bp, Artificial
  An artificially synthesized primer sequence for RT-PCR
cttgggtctgtaacaaagcattc

SEQ ID NO: 23 - DNA, 20 bp, Artificial
  An artificially synthesized primer sequence for RT-PCR
gatcaacatccacagcgaga

SEQ ID NO: 24 - DNA, 20 bp, Artificial
  An artificially synthesized primer sequence for RT-PCR
tgtcacagagccgaatacca

SEQ ID NO: 25 - RNA, 21 nt, Artificial
  An artificially synthesized oligonucleotide for dsRNA
gauaugccaucccagauuuuu

SEQ ID NO: 26 - RNA, 21 nt, Artificial
  An artificially synthesized oligonucleotide for dsRNA
aaaucugggauggcauaucuu

SEQ ID NO: 27 - RNA, 21 nt, Artificial
  An artificially synthesized oligonucleotide for dsRNA
gucaaauuccccaaauuaauu

SEQ ID NO: 28 - RNA, 21 nt, Artificial
  An artificially synthesized oligonucleotide for dsRNA
uuaauuuggggaauuugacuu

SEQ ID NO: 29 - RNA, 21 nt, Artificial
  An artificially synthesized oligonucleotide for dsRNA
guguccagaggccaauauuuu

SEQ ID NO: 30 - RNA, 21 nt, Artificial
  ′An artificially synthesized oligonucleotide for dsRNA
aauauuggccucuggacacuu

SEQ ID NO: 31 - RNA, 21 nt, Artificial
  An artificially synthesized oligonucleotide for dsRNA
ggcagggcuccaaaagacauu

SEQ ID NO: 32 - RNA, 21 nt, Artificial
  An artificially synthesized oligonucleotide for dsRNA
ugucuuuuggagcccugccuu

SEQ ID NO: 33 - RNA, 21 nt, Artificial
  An artificially synthesized oligonucleotide for dsRNA
ggagcccaucgguacagauuu

SEQ ID NO: 34 - RNA, 21 nt, Artificial
  An artificially synthesized oligonucleotide for dsRNA
aucuguaccgaugggcuccuu

SEQ ID NO: 35 - RNA, 21 nt, Artificial
  An artificially synthesized oligonucleotide for dsRNA
cggcggagccccaucaagauu

SEQ ID NO: 36 - RNA, 21 nt, Artificial
  An artificially synthesized oligonucleotide for dsRNA
ucuugauggggcuccgccguu

SEQ ID NO: 37 - RNA, 21 nt, Artificial
  An artificially synthesized oligonucleotide for dsRNA
gcggagccccaucaagaaauu

SEQ ID NO: 38 - RNA, 21 nt, Artificial
  ′An artificially synthesized oligonucleotide for dsRNA
uuucuugauggggcuccgcuu

SEQ ID NO: 39 - RNA, 21 nt, Artificial
  An artificially synthesized oligonucleotide for dsRNA
gaugugaagcugaugauguuu

SEQ ID NO: 40 - RNA, 21 nt, Artificial
  An artificially synthesized oligonucleotide for dsRNA
acaucaucagcuucacaucuu

SEQ ID NO: 41 - RNA, 21 nt, Artificial
  An artificially synthesized oligonucleotide for dsRNA
ugcugaccaucaagugccuuu

SEQ ID NO: 42 - RNA, 21 nt, Artificial
  An artificially synthesized oligonucleotide for dsRNA
aggcacuugauggucagcauu

SEQ ID NO: 43 - RNA, 21 nt, Artificial
  An artificially synthesized oligonucleotide for dsRNA
ccauaugcuggaggucuguuu

SEQ ID NO: 44 - RNA, 21 nt, Artificial
  An artificially synthesized oligonucleotide for dsRNA
acagaccuccagcauaugguu

SEQ ID NO: 45 - RNA, 21 nt, Artificial
  An artificially synthesized oligonucleotide for dsRNA
agagagagcugcacauguuuu

SEQ ID NO: 46 - RNA, 21 nt, Artificial
  An artificially synthesized oligonucleotide for dsRNA
aacaugugcagcucucucuuu

SEQ ID NO: 47 - DNA, 19 bp, Artificial
  A target sequence
gatatgccatcccagattt

SEQ ID NO: 48 - DNA, 19 bp, Artificial
  A target sequence
gtcaaattccccaaattaa

SEQ ID NO: 49 - DNA, 19 bp, Artificial
  A target sequence
gtgtccagaggccaatatt

SEQ ID NO: 50 - DNA, 19 bp, Artificial
  A target sequence
ggcagggctccaaaagaca

SEQ ID NO: 51 - DNA, 19 bp, Artificial
  A target sequence
ggagcccatcggtacagat

SEQ ID NO: 52 - DNA, 19 bp, Artificial
  A target sequence
cggcggagccccatcaaga

SEQ ID NO: 53 - DNA, 19 bp, Artificial
  A target sequence
gcggagccccatcaagaaa

SEQ ID NO: 54 - DNA, 19 bp, Artificial
  A target sequence
gatgtgaagctgatgatgt

SEQ ID NO: 55 - DNA, 19 bp, Artificial
  A target sequence
tgctgaccatcaagtgcct

SEQ ID NO: 56 - DNA, 19 bp, Artificial
  A target sequence
ccatatgctggaggtctgt

SEQ ID NO: 57 - DNA, 19 bp, Artificial
  A target sequence
agagagagctgcacatgtt

(19) United States
(12) Patent Application Publication
(10) Pub. No.: US 2010/0273855 A1
(43) Pub. Date: Oct. 28, 2010

(54) COMPOSITIONS AND METHODS OF TREATING CANCER

(75) Inventors: Akira Togashi, Kanagawa (JP); Ryutaro Tobita, Kanagawa (JP); Yuka Ishizaki, Kanagawa (JP); Akiko Konuma, Kanagawa (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ONCOTHERAPY SCIENCE, INC., Kawasaki-shi, Kanagawa (JP)

(21) Appl. No.: 12/666,253

(22) PCT Filed: Jun. 26, 2008
(86) PCT No.: PCT/JP2008/001665

§ 371 Date: Jun. 14, 2010

Related U.S. Application Data
(60) Provisional application No. 60/937,616, filed on Jun. 27, 2007.

(51) Int. Cl. A61K 31/713; C07H 21/00; C12N 15/63; A61P 35/00

(52) U.S. Cl. 514/44 A; 536/24.5; 435/320.1

(57) ABSTRACT

The invention features a method for treating cancer by administering a double-stranded nucleic acid molecule against a CX gene selected from the group consisting of C14orf78, MYBL2, UBE2S and UBE2T. The invention also features products, including the double-stranded nucleic acid molecules and vectors encoding them, as well as compositions comprising the molecules or vectors, useful in the provided methods. The methods of the invention are suited for the treatment of cancers including lung cancer, breast cancer, bladder cancer, esophagus cancer, prostate cancer, cholangiocellular carcinoma and testicular seminoma.

### CROSS-REFERENCE TO RELATED APPLICATIONS

The present application claims the benefit of U.S. Provisional Application No. 60/937,616, filed Jun. 27, 2007, the entire disclosure of which is hereby incorporated herein by reference.

### TECHNICAL FIELD

The present invention relates to the field of biological science, more specifically to the field of cancer research. In particular, the present invention relates to a double-stranded nucleic acid molecule which inhibits the expression of a CX gene selected from the group of C14orf78, MYBL2, UBE2S and UBE2T genes, and a composition comprising the same. The present invention further relates to methods of treating cancer using the molecules or compositions.

### BACKGROUND ART

### Pancreatic Cancer (Pancreatic Ductal Adenocarcinoma)

Pancreatic ductal adenocarcinoma (PDAC) is the forth leading cause of cancer death in the Western world and shows one of the worst mortality rates among malignancies, with a 5-year survival rate of only 4% (DiMagno EP et al., Gastroenterology 1999 December, 117(6): 1464-84; Zervos EE et al., Cancer Control 2004 January-February, 11(1): 23-31; Jemal A et al., CA Cancer J Clin 2003 January-February, 53(1): 5-26). Approximately 30,700 patients are diagnosed with pancreatic cancer in the United States alone, and nearly 30,000 will die of the disease (Jemal A et al., CA Cancer J Clin 2003 January-February, 53(1): 5-26). Because most PDAC patients are diagnosed at an advanced stage, none of the available therapies are effective. Surgical resection is the only possible cure at present, however 80% to 90% of PDAC patients who undergo surgery recur and die from this disease (DiMagno EP et al., Gastroenterology 1999 December, 117(6): 1464-84; Zervos EE et al., Cancer Control 2004 January-February, 11(1): 23-31). Some approaches in surgery and chemotherapy, including 5-fluorouracil (5-FU) or gemcitabine, with or without radiation, can improve patients' quality of life (DiMagno EP et al., Gastroenterology 1999 December, 117(6): 1464-84; Zervos EE et al., Cancer Control 2004 January-February, 11(1): 23-31). However, these treatments show only limited effect on long-term survival because PDACs are extremely aggressive and chemo-resistant. To overcome this almost hopeless situation, development of novel molecular therapies for PDAC through identification of molecular targets is an urgent priority.

Lung Cancer

Lung cancer is the leading cause of cancer deaths worldwide, and non small-cell lung cancer (NSCLC) accounts for nearly 80% of those cases (Greenlee R T et al., CA Cancer J Clin 2001 January-February, 51(1): 15-36). Many genetic alterations associated with development and progression of lung cancer have been reported, but the precise molecular mechanisms remain unclear (Sozzi G, Eur J Cancer 2001 October, 37 Suppl 7: S63-73). Within the last decade several newly developed chemotherapeutic agents such as paclitaxel, docetaxel, gemcitabine, and vinorelbine have begun to offer multiple choices for treatment of patients with advanced lung cancer; however, each of those regimens confers only a modest survival benefit compared with cisplatin-based therapies (Kelly K et al., J Clin Oncol 2001 Jul. 1, 19(13): 3210-8; Schiller J H et al., N Engl J Med 2002 Jan. 10, 346(2): 92-8). Hence, novel therapeutic strategies such as molecular-targeted drugs and antibodies and cancer vaccines are eagerly being sought.

Compared with other types of lung cancer, small-cell lung cancer (SCLC) has a greater tendency to be widely disseminated by the time of diagnosis and is highly aggressive, clinically characterized by rapid growth, frequent invasion, and metastasis (Ihde D C, N Engl J Med 1992 Nov. 12, 327(20): 1434-41). SCLC is a common type of lung cancer that is generally classified within the spectrum of neuroendocrine lung neoplasms, the origin of SCLC is thought to be derived from neural crest. It is well-known that SCLC initially may be sensitive to chemo- and radiotherapy, but unfortunately, many of them will become resistant to any therapy.

Breast Cancer

One million women worldwide are diagnosed with breast cancer every year. Estrogen receptor (ER)-positive breast cancers generally have a better prognosis because adjuvant hormonal therapy with anti-estrogen reagents such as tamoxifen or tremifen is usually effective regardless of age, menopausal status, axillary-node involvement, or tumor size. Estrogen deprivation therapy with a non-steroidal third-generation aromatase inhibitor is even more effective than tamoxifen for endocrine treatment of post-menopausal women with ER-positive advanced breast cancers (Nabholtz J M et al., J Clin Oncol 2000 Nov. 15, 18(22): 3758-67; Mouridsen H et al., J Clin Oncol 2001 May 15, 19(10): 2596-606). While these agents are of significant clinical value, the major limitation of endocrine therapy remains the nearly universal development of chemo-resistance. Most ER-positive breast cancers that respond initially to endocrine therapies acquire resistance to anti-estrogen therapy and convert to ER-negative tumors. Unfortunately, ER-negative breast cancers tend to be more aggressive as well as unresponsive to anti-estrogens (Goldhirsch A et al., J Clin Oncol 2003 Sep. 1, 21(17): 3357-65, Epub 2003 Jul. 7). Numerous targeted therapies are being investigated for this disease, including tyrosine kinase inhibitors (Gee J M et al., Endocrinology 2003 November, 144(11): 5105-17, Epub 2003 Aug. 7; Moulder SL & Arteaga CL, Clin Breast Cancer 2003 June, 4(2): 142-5; Okubo S et al., Br J Cancer 2004 Jan. 12, 90(1): 236-44; Schneeweiss A et al., Anticancer Drugs 2004 March, 15(3): 235-8; Warburton C et al., Clin Cancer Res 2004 Apr. 1, 10(7): 2512-24), however promising results have been achieved in only a limited number of patients thus far with some recipients suffering severe adverse reactions.

Bladder Cancer

Bladder cancer is the second most common genitourinary tumor in human populations, having an incidence of 261,000 new cases each year worldwide. Most bladder cancers present as superficial disease and are likely to recur in 50% to 75% of instances (Heney N M et al., J Urol 1983 December, 130(6): 1083-6). Thus, the ongoing prevalence of this cancer far exceeds its primary incidence. Moreover, although only 15% to 25% of these cases are likely to progress, an additional 25% of cases are invasive at initial presentation (Kaye K W & Lange P H, J Urol 1982 July, 128(1): 31-3). Therefore this cancer is requiring a high surveillance. Although radical cystectomy is considered currently the common treatment for patients with localized but muscle-invasive bladder cancer, about 50% of such patients develop metastases within 2 years after cystectomy and subsequently die of the disease (Sternberg C N, Ann Oncol 1995 February, 6(2): 113-26).

Esophagus Cancer

Cancer in the esophagus is a worldwide malignant neoplasm in particular in Pacific countries. Surgery remains the standard approach for treatment of patients with locoregional advanced disease that is resectable. Curative resection is feasible in 50% of cases, yet local or distant lesions are common after resection (Tepper J, J Clin Oncol 2000 February, 18(3): 453-4). The 5-year survival is only ˜30% for stage III and stage 1V patients undergoing surgery. Some adjuvant multimodality therapies have been attempted to control both local and systemic disease (Coia L R et al., J Clin Oncol, 2000 February, 18(3): 455-62; Pouliquen X et al., Ann Surg 1996 February, 223(2): 127-33). However, unresectable and relapsed esophageal cancers can be resistant to presently available chemotherapy or radiation therapy regimens, and there is almost no clear advantage of these regimens on overall survival. Consequently, development of a new effective therapeutic approach such as molecular-targeting therapy is needed to expand treatment modalities.

Prostate Cancer

Prostate cancer is the most common malignancy in males and the second leading cause of cancer-related death in the United States and Europe (Gronberg H, Lancet 2003 Mar. 8, 361(9360): 859-64), and frequency of prostate cancer has been increasing significantly in most developed countries probably due to prevalent western-style diet and the explosion of the aging population (Hsing A W & Devesa S S, Epidemiol Rev 2001, 23(1): 3-13; Feldman B J & Feldman D, Nat Rev Cancer 2001 October, 1(1): 34-45). Surgical and radiation therapies are effective to the localized disease, but nearly 30% of treated prostate cancer patients still suffer from the relapse of the disease (Han M et al., J Urol 2001 August, 166(2): 416-9; Isaacs W et al., Cancer Cell 2002 August, 2(2): 113-6). Most of the patients with relapsed or advanced disease respond well to androgen ablation therapy because prostate cancers are usually androgen-dependent at a relatively early stage. However, they often acquire androgen-independent phenotype and show no or very poor response to the androgen ablation therapy. No effective anticancer drug or therapy is presently available to the advanced or recurrent androgen-independent prostate cancer. Hence, development of new therapies based on the molecular mechanisms of prostate carcinogenesis or hormone refractory is urgently and eagerly required.

Testicular Seminoma

Although testicular germ cell tumors (TGCTs) account for around 1-2% of all cancers in males, they are the most common cancers found in males aged 20 to 40 year-old age group (Chaganti, R. et al. Cancer Res., 60: 1475-1482, 2000.), and the incidence has been markedly increasing over the past several decades (Bergstorm, R., et al. J. Natl. Cancer Inst., 88: 727-733, 1996, 3; Zheng, T., et al. Int. J. Cancer, 65: 723-729, 1996.). TGCTs are divided into two main histological types, the seminoma, which resembles the undifferentiated germ cells and the nonseminoma, which can resemble both embryonic and extra-embryonic tissues due to their ability to differentiate down either pathway (Smiraglia, D. J., et al. Oncogene, 21: 3909-3916, 2002.). Seminoma is the most common histologic testis tumor in TGCTs and account for approximately 60% to 65% of all TGCTs (Richie, J. P. et al. Cambell's Urology Seventh Edition, pp 2411-2452. Philadelphia: W.B Sauders Co., 1998). Currently, Alpha-fetoprotein (AFP), human beta-subunit chorionic gonadotropin (HCG beta) and lactic dehydrogenase (LDH) have been used as diagnostic tumor markers of TGCTs (Van Brussel, J. P. and Mikisch, G. H. J. BJU International, 83: 910-917, 1999). However, a specific therapeutic target for seminoma has not been identified.

Cholangiocellular Carcinoma

Cholangiocellular carcinoma is a malignant neoplasm arising from the biliary epithelium that was first described by Durand-Fardel in 1840. Today, it continues to defy diagnosis and treatment. It is difficult to diagnose in part because of its relative rarity, and because it is clinically silent until it becomes advanced disease with obstructive symptoms. The worldwide incidence of cholangiocellular carcinoma has risen over the past three decades. There is marked geographic variability in the prevalence of this disease, due in large part to regional environmental risk factors. Surgical resection remains the only curative treatment, and high priorities are improving diagnostic methods, and clinical staging for resection once the disease is suspected. A recent trend towards aggressive surgical management has improved outcomes. Chemotherapy, palliative stenting, and radiation are reserved for patients who are not resectable, those with recurrence after surgery, and those who decline surgical intervention. Recent trials using combination systemic chemotherapy and neoadjuvant chemoradiation are promising, but require further study.

Colon Cancer

Colon cancer is a leading cause of cancer deaths in developed countries. Specifically, more than 130,000 new cases of colorectal cancer in the United States are reported each year. Colon cancer represents about 15% of all cancers. Of these, approximately 5% are directly related to inherited genetic defects. Many patients have a diagnosis of pre-cancerous colon or rectal polyps before the onset of cancer. While many small colorectal polyps are benign, some types may progress to cancer. The most widely used screening test for colorectal cancer is colonoscopy. This method is used to visualize a suspicious growth and/or take a tissue biopsy. Typically, the tissue biopsy is histologically examined and a diagnosis delivered based on the microscopic appearance of the biopsied cells. However, this method is limited in that it yields subjective results and can not be used for very early detection of pre-cancerous states. The development of a sensitive, specific and convenient diagnostic system for detecting very early-stage colorectal cancers or pre-malignant lesions is highly desirable as it could ultimately eliminate this disease.

RNAi

RNA interference can be induced in a cell by different species of double-stranded nucleic acid molecules, including short interfering RNA (siRNA), e.g., double-stranded RNA (dsRNA) and short hairpin RNA (shRNA), and short interfering DNA/RNA (siD/RNA), e.g., double-stranded DNA/RNA (dsD/RNA) and short hairpin DNA/RNA (shD/RNA). In RNAi, one strand of double-stranded nucleic acid molecule has the polynucleotide sequence that is identical or substantially identical to the nucleotide sequence in the targeted gene transcript (mRNA) whereas the second strand of the double-stranded nucleic acid molecule has a complementary sequence thereto. Without wishing to be bound to theory, it is accepted that once the double-stranded nucleic acid molecules are introduced into a cell or are generated from longer double-stranded nucleic acid molecules in the cell by the RNaseIII like enzyme, the double-stranded nucleic acid molecule associates with a protein complex, known as the RNA-induced silencing complex (RISC). The RISC then guides the small double-stranded nucleic acid molecule to the mRNA where the two strands of the double-stranded nucleic acid molecule separate, the antisense strand associates with the mRNA and a nuclease cleaves the mRNA at the site where the antisense strand of the double-stranded nucleic acid molecule binds (Hammond S M et al., Nature 2000 Mar. 16, 404(6775): 293-6). The mRNA is subsequently further degraded by cellular nucleases. Short hairpin types have been shown to be potent RNAi triggers and in some instances maybe more effective than double-stranded nucleic acid molecules (Siolas D et al., Nat Biotechnol 2005 February, 23(2): 227-31, Epub 2004 Dec. 26). shRNAs may be produced by chemical synthesis as well as recombinant methods.

Recent years, a new approach of cancer therapy using gene-specific siRNA was carried out in clinical trials (Bumcrot D et al., Nat Chem Biol 2006 December, 2(12): 711-9). RNAi has earned a place among the major technology platforms (Putral L N et al., Drug News Perspect 2006 July-August, 19(6): 317-24; Frantz S, Nat Rev Drug Discov 2006 July, 5(7): 528-9; Dykxhoorn D M et al., Gene Ther 2006 March, 13(6): 541-52).

Atelocollagen, a Novel Delivery Tool for siRNA

Collagen is a triple helical fibrous protein observed in the various connective tissues. Atelocollagen obtained by pepsin treatment shows quite low immunogenicity because it is free from telopeptides involved in antigenicity (Stenzel K H, et al. Annu. Rev. Biophys Bioeng., 1974; 3: 231-53). Furthermore atelocollagen enhances cellular uptake, nuclease resistance and prolonged release of genes and oligonucleotides (Ochiya T, et al. Curr. Gene Ther., 2001; 1: 31-52). Atelocollagen has excellent properties which display low-toxicity and low-immunogenicity when it is transplanted in vivo (Ochiya T, et al. Curr. Gene Ther., 2001; 1: 31-52; Sano A, et al. Adv. Drug Deliv. Rev., 2003; 55: 1651-77). Recent studies of Ochiya et al. showed atelocollagen was available as a carrier of siRNA (Minakuchi Y, et al. Nucleic Acids Res. 2004; 32: e109; Takeshita F, et al. Proc Natl Acad Sci USA. 2005 Aug. 23; 102: 12177-82).

### Non Patent Citation 1: DiMagno EP et al., Gastroenterology 1999 December, 117(6): 1464-84

### Non Patent Citation 2: Zervos EE et al., Cancer Control 2004 January-February, 11(1): 23-31

### Non Patent Citation 3: Jemal A et al., CA Cancer J Clin 2003 January-February, 53(1): 5-26

### Non Patent Citation 4: Greenlee R T et al., CA Cancer J Clin 2001 January-February, 51(1): 15-36

### Non Patent Citation 5: Sozzi G, Eur J Cancer 2001 October, 37 Suppl 7: S63-73

### Non Patent Citation 6: Kelly K et al., J Clin Oncol 2001 Jul. 1, 19(13): 3210-8

### Non Patent Citation 7: Schiller J H et al., N Engl J Med 2002 Jan. 10, 346(2): 92-8

### Non Patent Citation 8: Ihde D C, N Engl J Med 1992 Nov. 12, 327(20): 1434-41

### Non Patent Citation 9: Nabholtz J M et al., J Clin Oncol 2000 Nov. 15, 18(22):3758-67

### Non Patent Citation 10: Mouridsen H et al., J Clin Oncol 2001 May 15, 19(10): 2596-606

### Non Patent Citation 11: Goldhirsch A et al., J Clin Oncol 2003 Sep. 1, 21(17): 3357-65, Epub 2003 Jul. 7

### Non Patent Citation 12: Gee J M et al., Endocrinology 2003 November, 144(11): 5105-17, Epub 2003 Aug. 7

### Non Patent Citation 13: Moulder S L & Arteaga C L, Clin Breast Cancer 2003 June, 4(2): 142-5

### Non Patent Citation 14: Okubo S et al., Br J Cancer 2004 Jan. 12, 90(1): 236-44

### Non Patent Citation 15: Schneeweiss A et al., Anticancer Drugs 2004 March, 15(3): 235-8

### Non Patent Citation 16: Warburton C et al., Clin Cancer Res 2004 Apr. 1, 10(7): 2512-24

### Non Patent Citation 17: Heney N M et al., J Urol 1983 December, 130(6): 1083-6

### Non Patent Citation 18: Kaye K W & Lange P H, J Urol 1982 July, 128(1): 31-3

### Non Patent Citation 19: Sternberg C N, Ann Oncol 1995 February, 6(2): 113-26

### Non Patent Citation 20: Tepper J, J Clin Oncol 2000 February, 18(3): 453-4

### Non Patent Citation 21: Coia L R et al., J Clin Oncol, 2000 February, 18(3): 455-62

### Non Patent Citation 22: Pouliquen X et al., Ann Surg 1996 February, 223(2): 127-33

### Non Patent Citation 23: Gronberg H, Lancet 2003 Mar. 8, 361(9360): 859-64

### Non Patent Citation 24: Hsing A W & Devesa S S, Epidemiol Rev 2001, 23(1): 3-13

### Non Patent Citation 25: Feldman B J & Feldman D, Nat Rev Cancer 2001 October, 1(1): 34-45

### Non Patent Citation 26: Han M et al., J Urol 2001 August, 166(2): 416-9

### Non Patent Citation 27: Isaacs W et al., Cancer Cell 2002 August, 2(2): 113-6

Non Patent Citation 28: Chaganti, R. et al. Cancer Res., 60: 1475-1482, 2000
Non Patent Citation 29: Bergstorm, R., et al. J. Natl. Cancer Inst., 88: 727-733, 1996, 3
Non Patent Citation 30: Zheng, T., et al. Int. J. Cancer, 65: 723-729, 1996.
Non Patent Citation 31: Smiraglia, D. J., et al. Oncogene, 21: 3909-3916, 2002.
Non Patent Citation 32: Richie, J. P. et al. Cambell's Urology Seventh Edition, pp 2411-2452. Philadelphia: W.B Sauders Co., 1998

### Non Patent Citation 33: Van Brussel, J. P. and Mikisch, G. H. J. BJU International, 83: 910-917, 1999

### Non Patent Citation 34: Hammond S M et al., Nature 2000 Mar. 16, 404(6775): 293-6

### Non Patent Citation 35: Siolas D et al., Nat Biotechnol 2005 February, 23(2): 227-31, Epub 2004 Dec. 26

### Non Patent Citation 36: Bumcrot D et al., Nat Chem Biol 2006 December, 2(12): 711-9

### Non Patent Citation 37: Putral L N et al., Drug News Perspect 2006 July-August, 19(6): 317-24

### Non Patent Citation 38: Frantz S, Nat Rev Drug Discov 2006 July, 5(7): 528-9

### Non Patent Citation 39: Dykxhoorn DM et al., Gene Ther 2006 March, 13(6): 541-52

Non Patent Citation 40: Stenzel K H, et al. Annu. Rev. Biophys Bioeng., 1974; 3: 231-53
Non Patent Citation 41: Ochiya T, et al. Curr. Gene Ther., 2001; 1: 31-52
Non Patent Citation 42: Sano A, et al. Adv. Drug Deliv. Rev., 2003; 55: 1651-77
Non Patent Citation 43: Minakuchi Y, et al. Nucleic Acids Res. 2004; 32: e109
Non Patent Citation 44: Takeshita F, et al. Proc Natl Acad Sci USA. 2005 Aug. 23; 102: 12177-82

### SUMMARY OF THE INVENTION

The present invention is based on the discovery that double-stranded nucleic acid molecules comprising specific sequences (in particular, SEQ ID NOs: 47 to 57) are effective for inhibiting cellular growth of various cancer cells, including those involved in pancreatic cancer, lung cancer, breast cancer, bladder cancer, esophagus cancer, prostate cancer, testicular seminoma, colon cancer and cholangiocellular carcinoma. Specifically, small interfering RNAs (siRNAs) targeting C14orf78, MYBL2, UBE2S and UBE2T genes are provided by the present invention.

According to an aspect of the present invention, the double-stranded nucleic acid molecules may be encoded in vectors and expressed from the vectors both in vivo and in vitro.

The double-stranded nucleic acid molecules and vectors of the present invention have the ability to inhibit cell growth of cells expressing a target gene (C14orf78, MYBL2, UBE2S or UBE2T genes). Thus, the invention provides methods for inhibiting cell growth and treating cancer by administering the double-stranded nucleic acid molecules or vectors of the present invention. Such methods include administering to a subject a composition comprising one or more of the double-stranded nucleic acid molecules or vectors.

Another aspect of the invention relates to compositions for treating cancer containing at least one of the double-stranded nucleic acid molecules or vectors of the present invention.

### DISCLOSURE OF INVENTION

### Profiles of Identified Therapeutic Candidates for Cancers

C14orf78 gene (Genbank Accession No. XM_{—}290629; SEQ ID NO: 1) encodes a giant protein (SEQ ID NO: 2; hereinafter, referred to as ‘C14orf78 protein’) with a molecular weight of 668 kDa. C14orf78 and AHNAK1 proteins belong to the same family as described previously (Komuro A et al., Proc Natl Acad Sci USA 2004 Mar. 23, 101(12): 4053-8, Epub 2004 Mar. 8). The size of AHNAK1 protein is a differentiation-related protein localized in interphase nuclei. A recent study reported that stimulation of cardiomyocytes by adrenergic agonists activated the phosphorylation of membrane-associated form AHNAK1 protein (Komuro A et al., Proc Natl Acad Sci USA 2004 Mar. 23, 101(12): 4053-8, Epub 2004 Mar. 8). The phosphorylated AHNAK1 protein co-precipitates with antibodies against two different subunits of the L-type voltage-regulated calcium channel, indicating that the protein is bound to calcium channels (Komuro A et al., Proc Natl Acad Sci USA 2004 Mar. 23, 101(12): 4053-8, Epub 2004 Mar. 8).

Another report found that no obvious abnormality could be detected in the phenotype of AHNAK1 knockout mice (Komuro A et al., Proc Natl Acad Sci USA 2004 Mar. 23, 101(12): 4053-8, Epub 2004 Mar. 8), indicating that AHNAK1 is not an essential factor for cellular proliferation and differentiation so far.

The protein encoded by MYBL2 gene (GenBank Accession No. NM_{—}002466; SEQ ID NO: 3 encoding SEQ ID NO: 4) functions as a transcription factor involved in cell cycle progression affecting cell proliferation, differentiation and apoptosis (Oh I H & Reddy E P, Oncogene 1999 May 13, 18(19): 3017-33; Weston K, Curr Opin Genet Dev 1998 February, 8(1): 76-81). MYBL2 protein also has been shown to act as either an activator or a repressor of gene transcription (Klempnauer K H & Sippel A E, EMBO J 1987 September, 6(9): 2719-25; Biedenkapp H et al., Nature 1988 Oct. 27, 335(6193): 835-7; Nomura N et al., Nucleic Acids Res 1988 Dec. 9, 16(23): 11075-89). MYBL2 gene expression has been previously reported to be limited to proliferating cells by an E2F-dependent mechanism, whereas the activity of the MYBL2 protein is stimulated by the CDK2/cyclin A complex in S-phase (Robinson C et al., Oncogene 1996 May 2, 12(9): 1855-64). The function of MYBL2 protein in mitosis relates at least partly to its ability to regulate cyclin B1 gene expression (Okada M et al., EMBO J 2002 Feb. 15, 21(4): 675-84.).

Both proteins encoded by UBE2S gene (GenBank Accession No. NM_{—}014501; SEQ ID NO: 5 encoding SEQ ID NO: 6) and UBE2T gene (GenBank Accession No. NM_{—}014176; SEQ ID NO: 7 encoding SEQ ID NO: 8) have one ubiquitin-conjugating enzyme E2 catalytic domain, and are thought to be ubiquitin-conjugating enzymes which contribute to the proteolytic pathway. Recent studies revealed that UBE2S protein, a putative ubiquitin E2 ligase, specifically targets pVHL (von Hippel-Lindau protein) for degradation; and over-expression of UBE2S gene remarkably promotes cell growth (Ohh M Cancer Cell 2006 August, 10(2): 95-7; Jung C R et al., Nat Med 2006 July, 12(7): 809-16, Epub 2006 Jul. 2).

pVHL functions as a substrate recognition module of ubiquitin ligase E3 complex which ubiquitinates hypoxia-inducible factor-1 alpha (HIF-1 alpha) under normoxic condition. HIF-1 alpha is normally degraded during normoxia, however, escapes from proteolytic machinery under hypoxia. This extraordinary accumulation of HIF-1 alpha evokes target gene activation which is involved in metabolic adaptation such as tumor vascularization, metabolization for cell survival, cell growth and differentiation (Semenza G L, Trends Mol Med 2001 August, 7(8): 345-50; Pugh C W & Ratcliffe P J, Nat Med 2003 June, 9(6): 677-84). Therefore, depletion of pVHL via ubiquitin pathway by UBE2S protein causes aberrant HIF-1 alpha accumulation, and consequently may promote cancer cell growth.

Protein ubiquitylation occurs through an ATP-dependent pathway. The first step requires ATP and ubiquitin is bound by a thioester linkage through its C-terminal glycine residue to an ubiquitin-activating enzyme (E1). Ubiquitin is then transferred to ubiquitin-conjugating enzymes (E2s) by trans-thiol esterification and then to a epsilon-amino group of a lysine residue in target protein, which is generally facilitated by an ubiquitin-protein ligase (E3). The conjugated ubiquitin itself may serve as an ubiquitylation substrate and repeated ubiquitylation leads to the formation of a polyubiquitin chain. Polyubiquitylated target proteins are transferred to the 26S proteasome. The ubiquitin-26S proteasome (UPS) pathway is a major mechanism in eukaryotic cells wherein normal and misfolded cytosolic or membrane proteins are degraded.

### DEFINITION

The words “a”, “an”, and “the” as used herein mean “at least one” unless otherwise specifically indicated.

The gene(s) that differentially expressed in cancer are collectively referred to herein as “CX gene(s)”, “CX nucleic acid(s)” or “CX polynucleotide(s)” and the corresponding encoded polypeptides are referred to as “CX polypeptide(s)” or “CX protein(s)”. In the present invention, a CX gene is selected from the group consisting of C14orf78 gene (may be referred to as “C14orf78”; GenBank Accession No. XM_{—}290629; SEQ ID NO: 1) encoding a giant protein (hereinafter referred to as “C14orf78 protein”; SEQ ID NO: 2), MYBL2 gene (may be referred to as “MYBL2”; GenBank Accession No. NM_{—}002466; SEQ ID NO: 3) encoding a protein having the sequence of SEQ ID NO: 4 (hereinafter referred to as “MYBL2 protein”), UBE2S gene (may be referred to as “UBE2S”; GenBank Accession No. NM_{—}014501; SEQ ID NO: 5) encoding a protein having the sequence of SEQ ID NO: 6 (hereinafter referred to as “UBE2S protein”) and UBE2T gene (may be referred to as “UBE2T”; GenBank Accession No. NM_{—}014176; SEQ ID NO: 7) encoding a protein having the sequence of SEQ ID NO: 8 (hereinafter referred to as “UBE2T protein”). Herein, these CX genes may also be referred to as “target gene(s)” and comprise at least one target sequence therein.

A target sequence is a nucleotide sequence within a CX gene, which will result in suppress of translation of the whole mRNA if a double-stranded nucleic acid molecule of the invention binds thereto. A nucleotide sequence within a CX gene can be determined to be a target sequence when a double-stranded polynucleotide comprising a sequence corresponding to the target sequence inhibits expression of the CX gene in a cell expressing the CX gene. According to the present invention, the following sequences were discovered to function as the target sequences:

C14orf78 gene:
nucleotides

### 13846-13864 (SEQ ID NO: 47),

### 13909-13927 (SEQ ID NO: 48),

### 14001-14019 (SEQ ID NO: 49) and

### 14647-14665 (SEQ ID NO: 50) of SEQ ID NO: 1;

MYBL2 gene:
nucleotides

### 977-995 (SEQ ID NO: 51),

### 1938-1956 (SEQ ID NO: 52),

### 1940-1958 (SEQ ID NO: 53) and

### 1995-2013 (SEQ ID NO: 54) of SEQ ID NO: 3;

UBE2S gene:
nucleotides

### 706-724 (SEQ ID NO: 55) and

### 528-546 (SEQ ID NO: 56) of SEQ ID NO: 5; and

UBE2T gene:
nucleotides

### 148-166 (SEQ ID NO: 57) of SEQ ID NO: 7.

As used herein, the term “organism” refers to any living entity composed of at least one cell. A living organism can be as simple as, for example, a single eukaryotic cell or as complex as a mammal, including a human being.

As used herein, the term “biological sample” refers to a whole organism or a subset of its tissues, cells or component parts (e.g., body fluids, including but not limited to blood, mucus, lymphatic fluid, synovial fluid, cerebrospinal fluid, saliva, amniotic fluid, amniotic cord blood, urine, vaginal fluid and semen). “Biological sample” further refers to a homogenate, lysate, extract, cell culture or tissue culture prepared from a whole organism or a subset of its cells, tissues or component parts, or a fraction or portion thereof. Lastly, “biological sample” refers to a medium, such as a nutrient broth or gel in which an organism has been propagated, which contains cellular components, such as proteins or polynucleotides.

The term “polynucleotide” and “oligonucleotide” are used interchangeably herein unless otherwise specifically indicated and are referred to by their commonly accepted single-letter codes. The terms apply to nucleic acid (nucleotide) polymers in which one or more nucleic acids are linked by ester bonding. The polynucleotide or oligonucleotide may be composed of DNA, RNA or a combination thereof.

As use herein, the term “isolated double-stranded nucleic acid molecule” refers to a nucleic acid molecule that inhibits expression of a target gene including, for example, short interfering RNA (siRNA; e.g., double-stranded ribonucleic acid (dsRNA) or small hairpin RNA (shRNA)) and short interfering DNA/RNA (siD/R-NA; e.g. double-stranded chimera of DNA and RNA (dsD/R-NA) or small hairpin chimera of DNA and RNA (shD/R-NA)).

As use herein, the term “siRNA” refers to a double-stranded RNA molecule which prevents translation of a target mRNA. Standard techniques of introducing siRNA into the cell are used, including those in which DNA is a template from which RNA is transcribed. The siRNA includes a CX sense nucleic acid sequence (also referred to as “sense strand”), a CX antisense nucleic acid sequence (also referred to as “antisense strand”) or both. The siRNA may be constructed such that a single transcript has both the sense and complementary antisense nucleic acid sequences of the target gene, e.g., a hairpin. The siRNA may either be a dsRNA or shRNA.

As used herein, the term “dsRNA” refers to a construct of two RNA molecules comprising complementary sequences to one another and that have annealed together via the complementary sequences to form a double-stranded RNA molecule. The nucleotide sequence of two strands may comprise not only the “sense” or “antisense” RNAs selected from a protein coding sequence of target gene sequence, but also RNA molecule having a nucleotide sequence selected from non-coding region of the target gene.

The term “shRNA”, as used herein, refers to an siRNA having a stem-loop structure, comprising a first and second regions complementary to one another, i.e., sense and antisense strands. The degree of complementarity and orientation of the regions being sufficient such that base pairing occurs between the regions, the first and second regions being joined by a loop region, the loop resulting from a lack of base pairing between nucleotides (or nucleotide analogs) within the loop region. The loop region of an shRNA is a single-stranded region intervening between the sense and antisense strands and may also be referred to as “intervening single-strand”.

As use herein, the term “siD/R-NA” refers to a double-stranded polynucleotide molecule which is composed of both RNA and DNA, and includes hybrids and chimeras of RNA and DNA and prevents translation of a target mRNA. Herein, a hybrid indicates a molecule wherein a polynucleotide composed of DNA and a polynucleotide composed of RNA hybridize to each other to form the double-stranded nucleic acid molecule; whereas a chimera indicates that one or both of the strands composing the double stranded molecule may contain RNA and DNA. Standard techniques of introducing siD/R-NA into the cell are used. In the present invention, such double-stranded nucleic acid molecule may refer to double-stranded molecule. The siD/R-NA includes a CX sense nucleic acid sequence (also referred to as “sense strand”), a CX antisense nucleic acid sequence (also referred to as “antisense strand”) or both. The siD/R-NA may be constructed such that a single transcript has both the sense and complementary antisense nucleic acid sequences from the target gene, e.g., a hairpin. The siD/R-NA may either be a dsD/R-NA or shD/R-NA.

As used herein, the term “dsD/R-NA” refers to a construct of two molecules comprising complementary sequences to one another and that have annealed together via the complementary sequences to form a double-stranded polynucleotide molecule. The nucleotide sequence of two strands may comprise not only the “sense” or “antisense” polynucleotides sequence selected from a protein coding sequence of target gene sequence, but also polynucleotide having a nucleotide sequence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, comprising a first and second regions complementary to one another, i.e., sense and antisense strands. The degree of complementarity and orientation of the regions being sufficient such that base pairing occurs between the regions, the first and second regions being joined by a loop region, the loop resulting from a lack of base pairing between nucleotides (or nucleotide analogs) within the loop region. The loop region of an shD/R-NA is a single-stranded region intervening between the sense and antisense strands and may also be referred to as “intervening single-strand”.

As used herein, an “isolated nucleic acid” is a nucleic acid removed from its original environment (e.g., the natural environment if naturally occurring) and thus, synthetically altered from its natural state. In the present invention, isolated nucleic acid includes DNA, RNA, and derivatives thereof.

The term “CX gene related disease”, as used herein, refers to a disease characterized by the over-expression of CX gene(s) compared with corresponding normal tissue, including, e.g. pancreatic cancer, lung cancer, breast cancer, bladder cancer, esophagus cancer, prostate cancer, testicular seminoma, colon cancer and cholangiocellular carcinoma.

Herein, inhibiting cell growth indicates that a cell naturally expressing a target gene proliferates at a lower rate or has decreased viability than an untreated cell. Cell growth can be measured by proliferation assays known in the art, for example, the assay using cell analyzer 1000.

Overview

In non-mammalian cells, double-stranded RNA (dsRNA) has been shown to exert strong and specific silencing effect on gene expression, which is referred to as RNA interference (RNAi) (Sharp P A, Genes Dev 1999 Jan. 15, 13(2): 139-41). A dsRNA is processed into 20 to 23 nucleotides, called small interfering RNA (siRNA), by an enzyme containing RNase III motif. The siRNA specifically targets complementary mRNA with a multicomponent nuclease complex (Hammond S M et al., Nature 2000 Mar. 16, 404(6775): 293-6; Hannon G J, Nature 2002 Jul. 11, 418(6894): 244-51). In mammalian cells, siRNA composed of 20 or 21-mer dsRNA with 19 complementary nucleotides and 3′ terminal non-complementary dimmers of thymidine or uridine, have been shown to possess gene specific knock-down effect without inducing global changes in gene expression (Elbashir S M et al., Nature 2001 May 24, 411(6836): 494-8). In addition, plasmids containing small nuclear RNA (snRNA) U6 or polymerase III H1-RNA promoter effectively produce such short RNA recruiting type III class of RNA polymerase III and thus can constitutively suppress its target mRNA (Miyagishi M & Taira K, Nat Biotechnol 2002 May, 20(5): 497-500; Brummelkamp T R et al., Science 2002 Apr. 19, 296(5567): 550-3, Epub 2002 Mar. 21).

The invention features methods of inhibiting cell growth. Cell growth is inhibited by contacting a cell with a double-stranded nucleic acid molecule against CX gene. Among the CX genes, C14orf78 was over-expressed (T/N ratio>=5) in 11 of 18 clinical pancreatic cancers, 14 of 25 clinical cholangiocellular carcinomas, and 10 of 37 non-small cell lung cancers; MYBL2 was revealed to be over-expressed in diverse spectrum of cancers, i.e., up-regulated (ratio>=5) in 18 of 34 clinical bladder cancers, 29 of 64 esophagus cancers, 18 of 37 non-small cell lung cancers (NSCLC), 6 of 18 clinical pancreatic cancers, and 14 of 15 small cell lung cancers (SCLC); UBE2S was over-expressed in all cases of SCLCs, 29 of 34 bladder cancers, 27 of 81 breast cancers, 18 of 59 prostate cancers, 11 of 48 colon cancers, 9 of 25 cholangiocellular carcinomas and 12 of 18 pancreatic cancers; and UBE2T showed also increased expression in various types of tumors, i.e., in 12 of 25 cholangiocellular carcinoma, 12 of 25 SCLCs, 23 of 34 bladder cancers, 28 of 81 breast cancers, 13 of 37 NSCLCs, 14 of 64 esophagus cancers, and 15 of 59 prostate cancers (Table 2). Growth of cells expressing the CX gene(s) can be inhibited by using double-stranded nucleic acid molecules of the present invention against respective target genes.

The method is used to alter gene expression in a cell in which expression of CX gene is up-regulated, e.g., as a result of malignant transformation of the cells. Binding of the double-stranded nucleic acid molecule to a transcript of CX gene in the target cell results in a reduction in CX protein production by the cell and inhibition of the cell growth.

Double-Stranded Nucleic Acid Molecule

A double-stranded nucleic acid molecule against a CX gene, which molecule hybridizes to target mRNA, decreases or inhibits production of the CX protein encoded by the CX gene by associating with the normally single-stranded mRNA transcript of the gene, thereby interfering with translation and thus, inhibiting expression of the protein. The expression of C14orf78 in PK-1 and Panc.02.03 pancreatic cancer cell lines, was inhibited by 4 different dsRNA (FIGS. 2a, b); the expression of MYBL2 in NSCLC (H358) and esophagus cancer (TE-9) cell lines was inhibited by 4 different dsRNA (FIGS. 3a, b); the expression of UBE2S in breast cancer (MCF7), pancreatic cancer (PK-1) and bladder cancer (SW780) cell lines was inhibited by 2 different dsRNA (FIG. 4a-c); and the expression of UBE2T in breast cancer (MCF7), NSCLC (A549), bladder cancer (SW780), and prostate cancer (DU-145) cell lines was inhibited by one dsRNA (FIG. 5a-d).

Therefore the present invention provides isolated double-stranded nucleic acid molecules having the property to inhibit expression of the CX gene when introduced into a cell expressing the gene. The target sequence of double-stranded nucleic acid molecule is designed by siRNA design algorithm mentioned below.

C14orf78 target sequence includes, for example, nucleotides

### 13846-13864 (SEQ ID NO: 47),

### 13909-13927 (SEQ ID NO: 48),

### 14001-14019 (SEQ ID NO: 49) or

### 14647-14665 (SEQ ID NO: 50) of SEQ ID NO: 1;

MYBL2 target sequence includes, for example, nucleotides

### 977-995 (SEQ ID NO: 51),

### 1938-1956 (SEQ ID NO: 52),

### 1940-1958 (SEQ ID NO: 53) or

### 1995-2013 (SEQ ID NO: 54) of SEQ ID NO: 3;

UBE2S target sequence includes, for example, nucleotides

### 706-724 (SEQ ID NO: 55) or

### 528-546 (SEQ ID NO: 56) of SEQ ID NO: 5; and

UBE2T target sequence includes, for example, nucleotides

### 148-166 (SEQ ID NO: 57) of SEQ ID NO: 7.

Specifically, the present invention provides the following double-stranded nucleic acid molecules [1] to [17]:

[1] An isolated double-stranded nucleic acid molecule, when introduced into a cell, inhibits expression of a CX gene and cell growth expressing the CX gene, wherein the CX gene is selected from the group consisting of C14orf78, MYBL2, UBE2S and UBE2T, which molecule comprises a sense strand and an antisense strand complementary thereto, hybridized to each other to form the double-stranded nucleic acid molecule and targets to a sequence selected from the group consisting of SEQ ID NOs: 47 to 57;

[2] The isolated double-stranded nucleic acid molecule of [1], wherein the sense strand comprises a sequence corresponding to a target sequence selected from the group consisting of SEQ ID NOs: 47 to 57;

[3] The double-stranded nucleic acid molecule of [2], which has a length of less than about 100 nucleotides;

[4] The double-stranded nucleic acid molecule of [3], which has a length of less than about 75 nucleotides;

[5] The double-stranded nucleic acid molecule of [4], which has a length of less than about 50 nucleotides;

[6] The double-stranded nucleic acid molecule of [5] which has a length of less than about 25 nucleotides;

[7] The double-stranded nucleic acid molecule of [6], which has a length of between about 19 and about 25 nucleotides;

[8] The double-stranded nucleic acid molecule of [1], which consists of a single polynucleotide comprising both the sense and antisense strands linked by an intervening single-strand;

[9] The double-stranded nucleic acid molecule of [8], which has the general formula 5′-[A]-[B]-[A′]-3′, wherein [A] is the sense strand comprising a sequence corresponding to a target sequence selected from the group consisting of SEQ ID NOs: 47 to 57, [B] is the intervening single-strand consisting of 3 to 23 nucleotides, and [A′] is the antisense strand comprising a complementary sequence to [A];

[10] The double-stranded nucleic acid molecule of [1], which comprises RNA;

[11] The double-stranded nucleic acid molecule of [1], which comprises both DNA and RNA;

[12] The double-stranded nucleic acid molecule of [11], which is a hybrid of a DNA polynucleotide and an RNA polynucleotide;

[13] The double-stranded nucleic acid molecule of [12] wherein the sense and the antisense strands consist of DNA and RNA, respectively;

[14] The double-stranded nucleic acid molecule of [11], which is a chimera of DNA and RNA;

[15] The double-stranded nucleic acid molecule of [14], wherein a region flanking to the 5′-end of the target sequence or the complementary sequence in the sense strand, and/or a region flanking to the 3′-end of the target sequence or the complementary sequence in the antisense strand consists of RNA;

[16] The double-stranded nucleic acid molecule of [15], wherein the flanking region consists of 9 to 13 nucleotides; and

[17] The double-stranded nucleic acid molecule of [1], which contains 3′ overhang. The double-stranded nucleic acid molecule of the present invention will be described in more detail below.

Methods for designing double-stranded nucleic acid molecules having the ability to inhibit target gene expression in cells are known. (See, for example, U.S. Pat. No. 6,506,559, herein incorporated by reference in its entirety). For example, a computer program for designing siRNAs is available from the Ambion website (http://www.ambion.com/techlib/misc/siRNA_finder.html).

The computer program selects target nucleotide sequences for double-stranded nucleic acid molecules based on the following protocol.

Selection of Target Sites

1. Beginning with the AUG start codon of the transcript, scan downstream for AA di-nucleotide sequences. Record the occurrence of each AA and the 3′ adjacent 19 nucleotides as potential siRNA target sites. Tuschl et al. recommend to avoid designing siRNA to the 5′ and 3′ untranslated regions (UTRs) and regions near the start codon (within 75 bases) as these may be richer in regulatory protein binding sites, and UTR-binding proteins and/or translation initiation complexes may interfere with binding of the siRNA endonuclease complex.

2. Compare the potential target sites to the appropriate genome database (human, mouse, rat, etc.) and eliminate from consideration any target sequences with significant homology to other coding sequences. Basically, BLAST, which can be found on the NCBI server at: www.ncbi.nlm.nih.gov/BLAST/, is used (Altschul SF et al., Nucleic Acids Res 1997 Sep. 1, 25(17): 3389-402).

3. Select qualifying target sequences for synthesis. Selecting several target sequences along the length of the gene to evaluate is typical.

By the protocol, the target sequence of the isolated double-stranded nucleic acid molecules of the present invention were designed as

nucleotides

13846-13864 (SEQ ID NO: 47),

13909-13927 (SEQ ID NO: 48),

14001-14019 (SEQ ID NO: 49) and

14647-14665 (SEQ ID NO: 50) of SEQ ID NO: 1 for C14orf78 gene;

nucleotides

977-995 (SEQ ID NO: 51),

1938-1956 (SEQ ID NO: 52),

1940-1958 (SEQ ID NO: 53) and

1995-2013 (SEQ ID NO: 54) of SEQ ID NO: 3 for MYBL2 gene;

nucleotides

706-724 (SEQ ID NO: 55) and

528-546 (SEQ ID NO: 56) of SEQ ID NO: 5 for UBE2S gene; and

nucleotides

148-166 (SEQ ID NO: 57) of SEQ ID NO: 7 for UBE2T gene.

Double-stranded nucleic acid molecules targeting the above-mentioned target sequences were respectively examined for their ability to suppress the growth of cells expressing the target genes. Therefore, the present invention provides double-stranded nucleic acid molecules targeting any of the sequences selected from the group of

nucleotides

13846-13864 (SEQ ID NO: 47),

13909-13927 (SEQ ID NO: 48),

14001-14019 (SEQ ID NO: 49) and

14647-14665 (SEQ ID NO: 50) of SEQ ID NO: 1 for C14orf78 gene;

nucleotides

977-995 (SEQ ID NO: 51),

1938-1956 (SEQ ID NO: 52),

1940-1958 (SEQ ID NO: 53) and

1995-2013 (SEQ ID NO: 54) of SEQ ID NO: 3 for MYBL2 gene;

nucleotides

706-724 (SEQ ID NO: 55) and

528-546 (SEQ ID NO: 56) of SEQ ID NO: 5 for UBE2S gene; and

nucleotides

148-166 (SEQ ID NO: 57) of SEQ ID NO: 7 for UBE2T gene.

The double-stranded nucleic acid molecule of the present invention is directed to a single target CX gene sequence or may be directed to a plurality of target CX gene sequences.

A double-stranded nucleic acid molecule of the present invention targeting one of the above-mentioned targeting sequences of a CX gene includes isolated polynucleotides that comprise any one of the sequences corresponding to the nucleic acid sequences of target sequences and/or complementary sequences to the target sequences. For instance, double-stranded nucleic acid molecules that targets the above-mentioned targeting sequences comprise the nucleotide sequence corresponding to the target sequence and complement thereof. In the present invention, when the double-stranded nucleic acid molecules comprises, or consists of RNA, nucleotide t (thymine) in the target sequence is replaced with u (uracil). Examples of oligonucleotides targeting C14orf78 gene include those comprising the sequence corresponding to the sequence of nucleotides 13846-13864 (SEQ ID NO: 47), 13909-13927 (SEQ ID NO: 48), 14001-14019 (SEQ ID NO: 49) or 14647-14665 (SEQ ID NO: 50) of SEQ ID NO: 1 and complementary sequences to these nucleotides; polynucleotides targeting MYBL2 gene include those comprising the sequence corresponding to the sequence of nucleotides 977-995 (SEQ ID NO: 51), 1938-1956 (SEQ ID NO: 52), 1940-1958 (SEQ ID NO: 53) or 1995-2013 (SEQ ID NO: 54) of SEQ ID NO: 3 and complementary sequences to these nucleotides; polynucleotides targeting UBE2S gene include those comprising the sequence corresponding to the sequence of nucleotides 706-724 (SEQ ID NO: 55) or 528-546 (SEQ ID NO: 56) of SEQ ID NO: 5 and complementary sequences to these nucleotides; and polynucleotides targeting UBE2T gene include those comprising the sequence corresponding to the sequence of nucleotides 148-166 (SEQ ID NO: 57) of SEQ ID NO: 7 and complementary sequences to these nucleotides. However, the present invention is not limited to these examples, and minor modifications in the aforementioned nucleic acid sequences are acceptable so long as the modified molecule retains the ability to suppress the expression of the CX gene. Herein, “minor modification” in a nucleic acid sequence indicates one, two or several substitution, deletion, addition or insertion of nucleic acids to the sequence.

According to the present invention, a double-stranded nucleic acid molecule of the present invention can be tested for its ability using the methods utilized in the Examples. In the Examples, the double-stranded nucleic acid molecules comprising sense strands or antisense strands complementary thereto of various portions of mRNA of the CX genes were tested in vitro for their ability to decrease production of the CX gene product in cancer cells (e.g., using the PK-1 cell line and Panc. 02. 03 cell line for pancreatic cancer cells, H358 cell line and A549 cell line for lung cancer cells, TE-9 cell line for esophagus cancer cells, MCF-7 cell line for breast cancer cell, SW780 cell line for bladder cancer cell and DU145 cell line for prostate cancer cell) according to standard methods. Furthermore, for example, reduction in a CX gene product in cells contacted with the candidate double-stranded nucleic acid molecule compared to cells cultured in the absence of the candidate molecule can be detected by, e.g., western blot analysis using antibodies against the CX protein or RT-PCR using primers for CX mRNA mentioned under Example 1, item “Semi-quantitative RT-PCR”. Sequences which decrease the production of a CX gene product in in vitro cell-based assays can then be tested for there inhibitory effects on cell growth. Sequences which inhibit cell growth in in vitro cell-based assay can then be tested for their in vivo ability using animals with cancer, e.g. nude mouse xenograft models, to confirm decreased production of the CX product and decreased cancer cell growth.

When the isolated polynucleotide is RNA or derivatives thereof, base “t” should be replaced with “u” in the nucleotide sequences. As used herein, the term “complementary” refers to Watson-Crick or Hoogsteen base pairing between nucleotides units of a polynucleotide, and the term “binding” means the physical or chemical interaction between two polynucleotides. When the polynucleotide comprises modified nucleotides and/or non-phosphodiester linkages, these polynucleotides may also bind each other as same manner. Generally, complementary polynucleotide sequences hybridize under appropriate conditions to form stable duplexes containing few or no mismatches. Furthermore, the sense strand and antisense strand of the isolated polynucleotide of the present invention can form double-stranded nucleic acid molecule or hairpin loop structure by the hybridization. In a preferred embodiment, such duplexes contain no more than 1 mismatch for every 10 matches. In an especially preferred embodiment, where the strands of the duplex are fully complementary, such duplexes contain no mismatches.

The polynucleotide is less than 15958 nucleotides in length for C14orf78, less than 2731 nucleotides in length for MYBL2, less than 1207 nucleotides in length for UBE2S, and less than 927 nucleotides in length for UBE2T. For example, the polynucleotide is less than 500, 200, 100, 75, 50, or 25 nucleotides in length for all of the genes. The isolated polynucleotides of the present invention are useful for forming double-stranded nucleic acid molecules against CX gene or preparing template DNAs encoding the double-stranded nucleic acid molecules. When the polynucleotides are used for forming double-stranded nucleic acid molecules, the polynucleotide may be longer than 19 nucleotides, preferably longer than 21 nucleotides, and more preferably has a length of between about 19 and 25 nucleotides.

The double-stranded nucleic acid molecules of the invention may contain one or more modified nucleotides and/or non-phosphodiester linkages. Chemical modifications well known in the art are capable of increasing stability, availability, and/or cell uptake of the double-stranded nucleic acid molecule. The skilled person will be aware of other types of chemical modification which may be incorporated into the present molecules (WO03/070744; WO2005/045037). In one embodiment, modifications can be used to provide improved resistance to degradation or improved uptake. Examples of such modifications include phosphorothioate linkages, 2′-O-methyl ribonucleotides (especially on the sense strand of a double-stranded nucleic acid molecule), 2′-deoxy-fluoro ribonucleotides, 2′-deoxy ribonucleotides, “universal base” nucleotides, 5′-C-methyl nucleotides, and inverted deoxyabasic residue incorporation (US20060122137). In another embodiment, modifications can be used to enhance the stability or to increase targeting efficiency of the double-stranded nucleic acid molecule. Modifications include chemical cross linking between the two complementary strands of a double-stranded nucleic acid molecule, chemical modification of a 3′ or 5′ terminus of a strand of a double-stranded nucleic acid molecule, sugar modifications, nucleobase modifications and/or backbone modifications, 2-fluoro modified ribonucleotides and 2′-deoxy ribonucleotides (WO2004/029212). In another embodiment, modifications can be used to increased or decreased affinity for the complementary nucleotides in the target mRNA and/or in the complementary double-stranded nucleic acid molecule strand (WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deza, 7-alkyi, or 7-alkenyi purine. In another embodiment, when the double-stranded nucleic acid molecule is a double-stranded nucleic acid molecule with a 3′ overhang, the 3′-terminal nucleotide overhanging nucleotides may be replaced by deoxyribonucleotides (Elbashir S M et al., Genes Dev 2001 Jan. 15, 15(2): 188-200). For further details, published documents such as US20060234970 are available. The present invention is not limited to these examples and any known chemical modifications may be employed for the double-stranded nucleic acid molecules of the present invention so long as the resulting molecule retains the ability to inhibit the expression of the target gene.

Furthermore, the double-stranded nucleic acid molecules of the invention may comprise both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type double-stranded nucleic acid molecule consisting of a DNA strand (polynucleotide) and an RNA strand (polynucleotide), a chimera type double-stranded nucleic acid molecule comprising both DNA and RNA on any or both of the single strands (polynucleotides), or the like may be formed for enhancing stability of the double-stranded nucleic acid molecule. The hybrid of a DNA strand and an RNA strand may be the hybrid in which either the sense strand is DNA and the antisense strand is RNA, or the opposite so long as it has an activity to inhibit expression of the target gene when introduced into a cell expressing the gene. Preferably, the sense strand polynucleotide is DNA and the antisense strand polynucleotide is RNA. Also, the chimera type double-stranded nucleic acid molecule may be either where both of the sense and antisense strands are composed of DNA and RNA, or where any one of the sense and antisense strands is composed of DNA and RNA so long as it has an activity to inhibit expression of the target gene when introduced into a cell expressing the gene. In order to enhance stability of the double-stranded nucleic acid molecule, the molecule preferably contains as much DNA as possible, whereas to induce inhibition of the target gene expression, the molecule is required to be RNA within a range to induce sufficient inhibition of the expression. As a preferred example of the chimera type double-stranded nucleic acid molecule, an upstream partial region (i.e., a region flanking to the target sequence or complementary sequence thereof within the sense or antisense strands) of the double-stranded nucleic acid molecule is RNA. Preferably, the upstream partial region indicates the 5′ side (5′-end) of the sense strand and the 3′ side (3′-end) of the antisense strand.

That is, in some embodiments, a region flanking to the 3′-end of the antisense strand, or both of a region flanking to the 5′-end of sense strand and a region flanking to the 3′-end of antisense strand consists of RNA. For instance, the chimera or hybrid type double-stranded nucleic acid molecule of the present invention comprise following combinations.

sense strand:
5′-[---DNA---]-3′
3′-(RNA)-[DNA]-5′
:antisense strand,
sense strand:
5′-(RNA)-[DNA]-3′
3′-(RNA)-[DNA]-5′
:antisense strand,
and
sense strand:
5′-(RNA)-[DNA]-3′
3′-(---RNA---)-5′
:antisense strand.

The upstream partial region preferably is a domain consisting of 9 to 13 nucleotides counted from the terminus of the target sequence or complementary sequence thereto within the sense or antisense strands of the double-stranded nucleic acid molecules. Moreover, preferred examples of such chimera type double-stranded nucleic acid molecules include those having a strand length of 19 to 21 nucleotides in which at least the upstream half region (5′ side region for the sense strand and 3′ side region for the antisense strand) of the polynucleotide is RNA and the other half is DNA. In such a chimera type double-stranded nucleic acid molecule, the effect to inhibit expression of the target gene is much higher when the entire antisense strand is RNA (US20050004064).

In the present invention, the double-stranded nucleic acid molecule may form a hairpin, such as a short hairpin RNA (shRNA) and short hairpin consisting of DNA and RNA (shD/R-NA). The shRNA or shD/R-NA is a sequence of RNA or mixture of RNA and DNA making a tight hairpin turn that can be used to silence gene expression via RNA interference. The shRNA or shD/R-NA comprises the sense target sequence and the antisense target sequence on a single strand wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

A loop sequence consisting of an arbitrary nucleotide sequence can be located between the sense and antisense sequence in order to form the hairpin loop structure. Thus, the present invention also provides a double-stranded nucleic acid molecule having the general formula 5′-[A]-[B]-[A′]-3′, wherein [A] is the sense strand comprising a sequence corresponding to a target sequence, [B] is an intervening single-strand and [A′] is the antisense strand comprising a complementary sequence to [A]. The target sequence may be selected from the group consisting of, for example, nucleotides

13846-13864 (SEQ ID NO: 47),

13909-13927 (SEQ ID NO: 48),

14001-14019 (SEQ ID NO: 49) or

14647-14665 (SEQ ID NO: 50) of SEQ ID NO: 1 for C14orf78;

nucleotides

977-995 (SEQ ID NO: 51),

1938-1956 (SEQ ID NO: 52),

1940-1958 (SEQ ID NO: 53) or

1995-2013 (SEQ ID NO: 54) of SEQ ID NO: 3 for MYBL2;

nucleotides

706-724 (SEQ ID NO: 55) or

528-546 (SEQ ID NO: 56) of SEQ ID NO: 5 for UBE2S; and

nucleotides

148-166 (SEQ ID NO: 57) of SEQ ID NO: 7 for UBE2T.

The present invention is not limited to these examples, and the target sequence in [A] may be modified sequences from these examples so long as the double-stranded nucleic acid molecule retains the ability to suppress the expression of the targeted CX gene. The region [A] hybridizes to [A′] to form a loop consisting of the region [B]. The intervening single-stranded portion [B], i.e., loop sequence may be preferably 3 to 23 nucleotides in length. The loop sequence, for example, can be selected from group consisting of following sequences (http://www.ambion.com/techlib/tb/tb_{—}506.html). Furthermore, loop sequence consisting of 23 nucleotides also provides active siRNA (Jacque J M et al., Nature 2002 Jul. 25, 418(6896): 435-8, Epub 2002 Jun. 26):

CCC, CCACC, or CCACACC: Jacque J M et al., Nature 2002 Jul. 25, 418(6896): 435-8, Epub 2002 Jun. 26;

UUCG: Lee NS et al., Nat Biotechnol 2002 May, 20(5): 500-5; Fruscoloni P et al., Proc Natl Acad Sci USA 2003 Feb. 18, 100(4): 1639-44, Epub 2003 Feb. 10; and

UUCAAGAGA: Dykxhoorn DM et al., Nat Rev Mol Cell Biol 2003 June, 4(6): 457-67.

Exemplary, preferable double-stranded nucleic acid molecules having hairpin loop structure of the present invention are shown below. In the following structure, the loop sequence can be selected from group consisting of AUG, CCC, UUCG, CCACC, CTCGAG, AAGCUU, CCACACC, and UUCAAGAGA; however, the present invention is not limited thereto:

gauaugccaucccagauuu-[B]-aaaucugggauggcauauc
(for target sequence SEQ ID NO: 47);
gucaaauuccccaaauuaa-[B]-uuaauuuggggaauuugac
(for target sequence SEQ ID NO: 48);
guguccagaggccaauauu-[B]-aauauuggccucuggacac
(for target sequence SEQ ID NO: 49);
ggcagggcuccaaaagaca-[B]-ugucuuuuggagcccugcc
(for target sequence SEQ ID NO: 50);
ggagcccaucgguacagau-[B]-aucuguaccgaugggcucc
(for target sequence SEQ ID NO: 51);
cggcggagccccaucaaga-[B]-ucuugauggggcuccgccg
(for target sequence SEQ ID NO: 52);
gcggagccccaucaagaaa-[B]-uuucuugauggggcuccgc
(for target sequence SEQ ID NO: 53);
gaugugaagcugaugaugu-[B]-acaucaucagcuucacauc
(for target sequence SEQ ID NO: 54);
ugcugaccaucaagugccu-[B]-aggcacuugauggucagca
(for target sequence SEQ ID NO: 55);
ccauaugcuggaggucugu-[B]-acagaccuccagcauaugg
(for target sequence SEQ ID NO: 56);
and
agagagagcugcacauguu-[B]-aacaugugcagcucucucu
(for target sequence SEQ ID NO: 57).

Furthermore, in order to enhance the inhibition activity of the double-stranded nucleic acid molecules, nucleotide “u” can be added to 3′end of the antisense strand of the target sequence, as 3′ overhangs. The number of “u”s to be added is at least 2, generally 2 to 10, preferably 2 to 5. The added “u”s form single strand at the 3′end of the antisense strand of the double-stranded nucleic acid molecule.

The method of preparing the double-stranded nucleic acid molecule is not particularly limited but it is preferable to use a chemical synthetic method known in the art. According to the chemical synthesis method, sense and antisense single-stranded polynucleotides are separately synthesized and then annealed together via an appropriate method to obtain a double-stranded nucleic acid molecule. Specific example for the annealing includes wherein the synthesized single-stranded polynucleotides are mixed in a molar ratio of preferably at least about 3:7, more preferably about 4:6, and most preferably substantially equimolar amount (i.e., a molar ratio of about 5:5). Next, the mixture is heated to a temperature at which double-stranded nucleic acid molecules dissociate and then is gradually cooled down. The annealed double-stranded polynucleotide can be purified by usually employed methods known in the art. Example of purification methods include methods utilizing agarose gel electrophoresis or wherein remaining single-stranded polynucleotides are optionally removed by, e.g., degradation with appropriate enzyme.

The regulatory sequences flanking the CX sequences may be identical or different, such that their expression can be modulated independently, or in a temporal or spatial manner. The double-stranded nucleic acid molecules can be transcribed intracellularly by cloning the CX gene templates into a vector containing, e.g., a RNA pol III transcription unit from the small nuclear RNA (snRNA) U6 or the human H1 RNA promoter.

Vector

Also included in the invention is a vector containing one or more of the double-stranded nucleic acid molecules described herein, and a cell containing the vector. A vector of the present invention preferably encodes a double-stranded nucleic acid molecule of the present invention in an expressible form. Herein, the phrase “in an expressible form” indicates that the vector, when introduced into a cell, will express the molecule. In a preferred embodiment, the vector includes regulatory elements necessary for expression of the double-stranded nucleic acid molecule. Such vectors of the present invention may be used for producing the present double-stranded nucleic acid molecules, or directly as an active ingredient for treating cancer.

Vectors of the present invention can be produced, for example, by cloning a CX sequence into an expression vector so that regulatory sequences are operatively-linked to the CX sequence in a manner to allow expression (by transcription of the DNA molecule) of both strands (Lee NS et al., Nat Biotechnol 2002 May, 20(5): 500-5). For example, RNA molecule that is the antisense to mRNA is transcribed by a first promoter (e.g., a promoter sequence flanking to the 3′ end of the cloned DNA) and RNA molecule that is the sense strand to the mRNA is transcribed by a second promoter (e.g., a promoter sequence flanking to the 5′ end of the cloned DNA). The sense and antisense strands hybridize in vivo to generate a double-stranded nucleic acid molecule constructs for silencing of the gene. Alternatively, two vectors construct respectively encoding the sense and antisense strands of the double-stranded nucleic acid molecule are utilized to respectively express the sense and anti-sense strands and then forming a double-stranded nucleic acid molecule construct. Furthermore, the cloned sequence may encode a construct having a secondary structure (e.g., hairpin); namely, a single transcript of a vector contains both the sense and complementary antisense sequences of the target gene.

The vectors of the present invention may also be equipped so to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687).

The vectors of the present invention may be, for example, viral or bacterial vectors. Examples of expression vectors include attenuated viral hosts, such as vaccinia or fowlpox (see, e.g., U.S. Pat. No. 4,722,848). This approach involves the use of vaccinia virus, e.g., as a vector to express nucleotide sequences that encode the double-stranded nucleic acid molecule. Upon introduction into a cell expressing the target gene, the recombinant vaccinia virus expresses the molecule and thereby suppresses the proliferation of the cell. Another example of useable vector includes Bacille Calmette Guerin (BCG). BCG vectors are described in Stover et al., Nature 1991, 351: 456-60. A wide variety of other vectors are useful for therapeutic administration and production of the double-stranded nucleic acid molecules; examples include adeno and adeno-associated virus vectors, retroviral vectors, Salmonella typhi vectors, detoxified anthrax toxin vectors, and the like. See, e.g., Shata et al., Mol Med Today 2000, 6: 66-71; Shedlock et al., J Leukoc Biol 2000, 68: 793-806; and Hipp et al., In Vivo 2000, 14: 571-85.

Methods of Treating Cancer

In present invention, 4 different dsRNA for C14orf78, 4 different dsRNA for MYBL2, 2 different dsRNA for UBE2S and one dsRNA for UBE2T were constructed to test for their ability to inhibit cell growth. The four dsRNA for C14orf78 all effectively knocked down the expression of the gene in the cell expressing the gene, e.g. PK-1 and Panc.02.03, coincided with suppression of cell proliferation (FIGS. 2a, b), while no significant alteration was observed with these dsRNAs in SK-BR-3, a C14orf78 non-expressing cell line (FIG. 2c). The four dsRNA for MYBL2 all significantly decreased the expression level and cell growth activity in the cell expressing the gene, e.g. NSCLC (H358) and esophagus cancer (TE-9) cell lines (FIGS. 3a, b), while no detectable growth inhibition was observed in normal small airway epithelial cell (SAEC), a MYBL2 non-expressing cell line (FIG. 3c). The two dsRNA for UBE2S significantly decreased the expression level and cell viability in the cell expressing the gene, e.g. breast cancer (MCF7), pancreatic cancer (PK-1) and bladder cancer (SW780) cell lines (FIG. 4a-c) and one dsRNA for UBE2T effectively suppressed expression of the gene in the cell expressing the gene, e.g. breast cancer (MCF7), NSCLC (A549), bladder cancer (SW780), and prostate cancer (DU-145) cell lines (FIG. 5a-d); while no detectable growth inhibition was observed in HMEC (normal mammary epithelial cell), a non-expressing cell line of both UBE2S and UBE2T (FIG. 4d, 5e). Therefore, treatment with all of dsRNAs against a CX gene effectively inhibited the development of cancer in vivo (FIGS. 6a and b).

Such ability of the present double-stranded nucleic acid molecules and vectors to inhibit cell growth of cancerous cell indicates that they can be used for methods for treating cancer. Thus, the present invention provides methods to treat patients with cancers characterized as over-expressing a CX gene by administering a double-stranded nucleic acid molecule against the CX gene or a vector expressing the molecule.

In fact, it was confirmed that the CX genes were over-expression in cancer tissues with comparing to in corresponding normal tissues. For example, C14orf78 was overexpressed (T/N ratio>=5) in clinical samples; 11 of 18 pancreatic cancer, 14 of 25 cholangiocellular carcinomas and 10 of 37 non-small cell lung cancers; MYBL2 was revealed to be over-expressed in diverse spectrum of cancers, i.e., up-regulated (ratio>=5) in 6 of 18 pancreatic cancers, 18 of 34 clinical bladder cancers, 29 of 64 esophagus cancers, 18 of 37 non-small cell lung cancers (NSCLC), and 14 of 15 small cell lung cancers (SCLC); UBE2S was over-expressed in clinical samples; all cases of SCLCs, 29 of 34 bladder cancers, 27 of 81 breast cancers, 9 of 25 cholangiocellular carcinomas, 18 of 59 prostate cancers, 11 of 48 colon cancers, and 12 of 18 pancreatic cancers; and a similar protein to UBE2S, ubiquitin E2 ligase like UBE2T gene also showed increased expression in various type of cancers, i.e., in 12 of 25 cholangiocellular carcinoma, 12 of 15 SCLCs, 23 of 34 bladder cancers, 28 of 81 breast cancers, 13 of 37 NSCLCs, 14 of 64 esophagus cancer and 15 of 59 prostate cancers (Table 2).

In the present invention, CX genes that an inhibition effect of cell growth or cell proliferation was induced by suppression the expression level thereof are identified. Cell growth of cells expressing such genes may be inhibited by suppressing the expression of these genes. It was reported that CX genes according to the present invention are up-regulated in some cancers as follows:

C14orf78

pancreatic cancer (WO2004/31412)

MYBL2

bladder cancer (WO2006/085684)

esophagus cancer (WO2007/013671)

NSCLC (WO2004/031413)

pancreatic cancer (WO2004/31412)

SCLC (WO2007/013665)

testicular seminoma (WO2004/031410)

UBE2S

bladder cancer (WO2006/085684)

breast cancer (WO2005/028676)

pancreatic cancer (WO2004/31412)

prostate cancer (WO2004/031414)

SCLC (WO2007/013665)

UBE2T

bladder cancer (WO2006/085684)

breast cancer (WO2005/028676)

esophagus cancer (WO2007/013671)

NSCLC (WO2004/031413)

SCLC (WO2007/031413)

Accordingly, in preferable embodiments, the present invention provides a method for treating or preventing these cancers by inhibiting CX genes selected from group consisting of C14orf78, MYB2L, UBE2S, and UBE2T.

For example, the present invention provides a method for treating a cancer selected from the group consisting of pancreatic cancer, cholangiocellular carcinoma, and non-small cell lung cancer comprising the step of administering at least one isolated double-stranded nucleic acid molecule comprising a sense strand and antisense strand complementary thereto, hybridized to each other to form the double-stranded nucleic acid molecule and, wherein the sense strand comprises a sequence corresponding to a target sequence selected from the group consisting of SEQ ID Nos: 47 to 50 (for C14orf78).

The present invention further provides a method for treating a cancer selected from the group consisting of pancreatic cancer, non-small cell lung cancer, small cell lung cancer, bladder cancer, esophagus cancer and testicular siminoma, comprising the step of administering at least one isolated double-stranded nucleic acid molecule comprising a sense strand and antisense strand complementary thereto, hybridized to each other to form the double-stranded nucleic acid molecule and, wherein the sense strand comprising a sequence corresponding to a target sequence selected from the group consisting of SEQ ID NOs: 51 to 54 (for MYBL2).

Alternatively, the present invention also provides a method for treating a cancer selected from group consisting of pancreatic cancer, breast cancer, small cell lung cancer, bladder cancer, cholangiocellular carcinoma, colon cancer and prostate cancer, comprising the step of administering at least one isolated double-stranded nucleic acid molecule comprising a sense strand and an antisense strand complementary thereto, hybridized to each other to form the double-stranded nucleic acid molecule and, wherein the sense strand comprises a target sequence selected from the group consisting of SEQ ID NOs: 55 to 56 (for UBE2S).

Further, the present invention also provides a method for treating a cancer selected from the group consisting of breast cancer, non-small cell lung cancer, small cell lung cancer, bladder cancer, cholangiocellular carcinoma, prostate cancer and esophagus cancer, comprising the step of administering at least one isolated double-stranded nucleic acid molecule comprising a sense strand and an antisense strand complementary thereto, hybridized to each other to form the double-stranded nucleic acid molecule and, wherein the sense strand comprises a target sequence SEQ ID NO: 57 (for UBE2T).

Therefore, the method of the present invention may be used to inhibit expression of a CX gene in patients suffering from or at risk of developing CX gene related disease, for example, pancreatic cancer, non-small cell lung cancer, small cell lung cancer, breast cancer, bladder cancer, esophagus cancer, prostate cancer, colon cancer and/or cholangiocellular carcinoma. Preferably, double-stranded nucleic acid molecules against C14orf78 and vectors expressing them can be used for the treatment of pancreatic cancer, cholangiocellular carcinoma and/or non-small cell lung cancer; those against MYBL2 and vectors expressing them can be used for the treatment of bladder cancer, esophagus cancer, testicular seminoma, non-small cell lung cancer, pancreatic cancer and/or small cell lung cancer; those against UBES2 and vectors expressing them can be used for the treatment of small cell lung cancer, bladder cancer, breast cancer, cholangiocellular carcinoma, prostate cancer, colon cancer and/or pancreatic cancer; and those against UBE2T and vectors expressing them can be used for the treatment of cholangiocellular carcinoma, non-small cell lung cancer, small cell lung cancer, bladder cancer, breast cancer, esophagus cancer and/or prostate cancer.

Specifically, the present invention provides the following methods [1] to [29]:

[1] A method for treating cancer comprising the step of administering at least one isolated double-stranded nucleic acid molecule inhibiting the expression of a CX gene in a cell, which over-expresses the gene, wherein the CX gene is selected from the group consisting of C14orf78, MYBL2, UBE2S and UBE2T, which molecule comprises a sense strand and an antisense strand complementary thereto, hybridized to each other to form the double-stranded nucleic acid molecule and targets to a sequence selected from the group consisting of SEQ ID NOs: 47 to 57;

[2] The method of [1], wherein the sense strand comprises a sequence corresponding to a target sequence selected from the group consisting of SEQ ID NOs: 47 to 57;

[3] The method of [1], wherein the cell is a cancer cell;

[4] The method of [1], wherein the cancer to be treated is selected from the group of pancreatic cancer, lung cancer, breast cancer, bladder cancer, esophagus cancer, testicular seminoma, prostate cancer, colon cancer or cholangiocellular carcinoma;

[5] The method of [4], wherein the lung cancer is non-small lung cancer or small lung cancer;

[6] The method of [1], wherein the cancer to be treated is selected from the group of pancreatic cancer, cholangiocellular carcinoma or non-small cell lung cancer, when the selected CX gene is C14orf78;

[7] The method of [1], wherein the cancer to be treated is selected from the group of pancreatic cancer, non-small lung cancer, small lung cancer, bladder cancer, esophagus cancer or testicular seminoma, when the selected CX gene is MYBL2;

[8] The method of [1], wherein the cancer to be treated is selected from the group of pancreatic cancer, breast cancer, small lung cancer, bladder cancer, cholangiocellular carcinoma, prostate cancer or colon cancer, when the selected CX gene is UBE2S;

[9] The method of [1], wherein the cancer to be treated is selected from the group of breast cancer, non-small lung cancer, small lung cancer, bladder cancer, cholangiocellular carcinoma, prostate cancer or esophagus cancer, when the selected CX gene is UBE2T;

[10] The method of [1], wherein plural kinds of the double-stranded nucleic acid molecules are administered;

[11] The method of [10], wherein the plural kinds of the double-stranded nucleic acid molecules target the same gene;

[12] The method of [2], wherein the double-stranded nucleic acid molecule has a length of less than about 100 nucleotides;

[13] The method of [12], wherein the double-stranded nucleic acid molecule has a length of less than about 75 nucleotides;

[14] The method of [13], wherein the double-stranded nucleic acid molecule has a length of less than about 50 nucleotides;

[15] The method of [14], wherein the double-stranded nucleic acid molecule has a length of less than about 25 nucleotides;

[16] The method of [15], wherein the double-stranded nucleic acid molecule has a length of between about 19 and about 25 nucleotides in length;

[17] The method of [1], wherein the double-stranded nucleic acid molecule consists of a single polynucleotide comprising both the sense strand and the antisense strand linked by an intervening single-strand;

[18] The method of [17], wherein the double-stranded nucleic acid molecule has the general formula 5′-[A]-[B]-[A′]-3′, wherein [A] is the sense strand comprising a sequence corresponding to a target sequence selected from the group consisting of SEQ ID NOs: 47 to 57, [B] is the intervening single strand consisting of 3 to 23 nucleotides, and [A′] is the antisense strand comprising a complementary sequence to [A];

[19] The method of [1], wherein the double-stranded nucleic acid molecule comprises RNA;

[20] The method of [1], wherein the double-stranded nucleic acid molecule comprises both DNA and RNA;

[21] The method of [20], wherein the double-stranded nucleic acid molecule is a hybrid of a DNA polynucleotide and an RNA polynucleotide;

[22] The method of [21] wherein the sense and antisense strand polynucleotides consist of DNA and RNA, respectively;

[23] The method of [20], wherein the double-stranded nucleic acid molecule is a chimera of DNA and RNA;

[24] The method of [23], wherein a region flanking to the 5′-end of one or both of the sense and antisense polynucleotides consist of RNA;

[25] The method of [24], wherein the flanking region consists of 9 to 13 nucleotides;

[26] The method of [1], wherein the double-stranded nucleic acid molecule contains 3′ overhangs;

[27] The method of [1], wherein the double-stranded nucleic acid molecule is encoded by a vector;

[28] The method of [27], wherein the double-stranded nucleic acid molecule encoded by the vector has the general formula 5′-[A]-[B]-[A′]-3′, wherein [A] is the sense strand comprising a sequence corresponding to a target sequence selected from the group consisting of SEQ ID NOs: 47 to 57, [B] is a intervening single-strand consisting of 3 to 23 nucleotides, and [A′] is the antisense strand comprising a complementary sequence to [A]; and

[29] The method of [1], wherein the double-stranded nucleic acid molecule is contained in a composition which comprises in addition to the molecule a transfection-enhancing agent and pharmaceutically acceptable carrier.

The method of the present invention will be described in more detail below.

The growth of cells expressing a CX gene is inhibited by contacting the cells with a double-stranded nucleic acid molecule against the CX gene, a vector expressing the molecule or a composition comprising the same. The cell is further contacted with a transfection agent. Suitable transfection agents are known in the art. The phrase “inhibition of cell growth” indicates that the cell proliferates at a lower rate or has decreased viability compared to a cell not exposed to the molecule. Cell growth may be measured by methods known in the art, e.g., using Cell Analyzer 1000 and the MTT cell proliferation assay.

The growth of any kind of cell may be suppressed according to the present method so long as the cell expresses or over-expresses the target gene of the double-stranded nucleic acid molecule of the present invention. Exemplary cells include cancer cells, more specifically pancreatic cancer cells, lung cancer cells, breast cancer cells, bladder cancer cells, esophagus cancer cells, prostate cancer cells, testicular seminoma cells, colon cancer cells and cholangiocellular carcinoma cells.

Thus, patients suffering from or at risk of developing disease related to C14orf78, MYBL2, UBE2S or UBE2T may be treated by administering at least one of the present double-stranded nucleic acid molecules, at least one vector expressing at least one of the molecules or at least one composition comprising at least one of the molecules. For example, patients of cancer, specifically pancreatic cancer, lung cancer, breast cancer, bladder cancer, esophagus cancer, prostate cancer, testicular seminoma, colon cancer and/or cholangiocellular carcinoma may be treated according to the present methods. The type of cancer may be identified by standard methods according to the particular type of tumor to be diagnosed. Pancreatic cancer may be diagnosed, for example, by magnetic resonance imaging, computerized axial tomography ultrasound or biopsy. Lung cancer may be diagnosed, for example, by Chest radiograph, computed tomography, magnetic resonance imaging, bronchoscopy, needle biopsy or bone scan. Breast cancer may be diagnosed, for example, by clinical examination, imaging procedures (e.g., mammogram, breast ultrasound, magnetic resonance imaging) or biopsy. Bladder cancer may be diagnosed, for example, NMP22(registered trademark) BladderChek(registered trademark), urinalysis, urine cytology or urine culture. Esophagus cancer may be diagnosed, for example, by needle aspiration, biopsy, blood tests or imaging tests esophagoscopy. Testicular seminoma or prostate cancer may be diagnosed, for example, by Digital rectal examination, transrectal ultrasound, prostate specific antigen (PSA) and prostate acid phosphatase (PAP) Tests, tumor Biopsy or bone scan. Cholangiocellular carcinoma may be diagnosed, for example, by enlargement of the liver, tomography, ultrasound or biopsy. Colon cancer may be diagnosed, for example, by blood in stool, colonoscopy, flexible sigmoidoscopy, CEA Assay, double contrast barium enema CT Scan, tomography or biopsy. More preferably, patients treated by the methods of the present invention are selected by detecting the expression of CX genes in a biopsy from the patient by RT-PCR or immunoassay. Preferably, before the treatment of the present invention, the biopsy specimen from the subject is confirmed for CX gene over-expression by methods known in the art, for example, immunohistochemical analysis or RT-PCR.

According to the present method to inhibit cell growth and thereby treating cancer, when administering plural kinds of the double-stranded nucleic acid molecules (or vectors expressing or compositions containing the same), each of the molecules may be directed to the same target sequence, or different target sequences within the same CX gene or on different CX genes. For example, the method may utilize double-stranded nucleic acid molecules directed to one, two, three or four of the CX genes. Alternatively, for example, the method may utilize double-stranded nucleic acid molecules directed to one, two, three, four, five or more target sequences within the same CX gene.

For inhibiting cell growth, a double-stranded nucleic acid molecule of present invention may be directly introduced into the cells in a form to achieve binding of the molecule with corresponding mRNA transcripts. Alternatively, as described above, a DNA encoding the double-stranded nucleic acid molecule may be introduced into cells as a vector. For introducing the double-stranded nucleic acid molecules and vectors into the cells, transfection-enhancing agent, such as FuGENE (Roche diagnostics), Lipofectamine 2000 (Invitrogen), Oligofectamine (Invitrogen), and Nucleofector (Wako pure Chemical), may be employed.

A treatment is determined efficacious if it leads to clinical benefit such as, reduction in expression of a CX gene, a decrease in size or inhibition of an expansion, prevalence, or metastatic potential of the cancer in the subject. When the treatment is applied prophylactically, “efficacious” means that it retards or prevents cancers from forming or prevents or alleviates a clinical symptom of cancer. Efficaciousness is determined in association with any known method for diagnosing or treating the particular tumor type.

It is understood that the double-stranded nucleic acid molecule of the invention degrades the target mRNA (of C14orf78, MYBL2, UBE2S or UBE2T) in substoichiometric amounts. Without wishing to be bound by any theory, it is believed that the double-stranded nucleic acid molecule of the invention causes degradation of the target mRNA in a catalytic manner. Thus, compared to standard cancer therapies, significantly less a double-stranded nucleic acid molecule needs to be delivered at or near the site of cancer to exert therapeutic effect.

One skilled in the art can readily determine an effective amount of the double-stranded nucleic acid molecule of the invention to be administered to a given subject, by taking into account factors such as body weight, age, sex, type of disease, symptoms and other conditions of the subject; the route of administration; and whether the administration is regional or systemic. Generally, an effective amount of the double-stranded nucleic acid molecule of the invention comprises an intercellular concentration at or near the cancer site of from about 1 nano-molar (nM) to about 100 nM, preferably from about 2 nM to about 50 nM, more preferably from about 2.5 nM to about 10 nM. It is contemplated that greater or smaller amounts of the double-stranded nucleic acid molecule can be administered.

The present methods can be used to inhibit the growth or metastasis of cancer; for example pancreatic cancer, lung cancer, breast cancer, bladder cancer, esophagus cancer, prostate cancer, testicular seminoma, colon cancer and cholangiocellular carcinoma. In particular, a double-stranded nucleic acid molecule comprising a target sequence of C14orf78 (i.e., SEQ ID NOs: 47 to 50) is particularly preferred for the treatment of pancreatic cancer, cholangiocellular carcinoma and non-small cell lung cancer; those comprising a target sequence of MYBL2 (i.e., SEQ ID NOs: 51 to 54) is particularly preferred for the treatment of pancreatic cancer, non-small lung cancer, small lung cancer, bladder cancer, esophagus cancer and testicular seminoma; those comprising a target sequence of UBE2S (i.e., SEQ ID NOs: 55 and 56) is particularly preferred for the treatment of pancreatic cancer, breast cancer, small lung cancer, bladder cancer, cholangiocellular carcinoma, prostate cancer and colon cancer; and those comprising a target sequence of UBE2T (i.e., SEQ ID NO: 55) is particularly preferred for the treatment of breast cancer, cholangiocellular carcinoma, non-small lung cancer, small lung cancer, bladder cancer, prostate cancer and esophagus cancer.

For treating cancer, the double-stranded nucleic acid molecule of the invention can also be administered to a subject in combination with a pharmaceutical agent different from the double-stranded nucleic acid molecule. Alternatively, the double-stranded nucleic acid molecule of the invention can be administered to a subject in combination with another therapeutic method designed to treat cancer. For example, the double-stranded nucleic acid molecule of the invention can be administered in combination with therapeutic methods currently employed for treating cancer or preventing cancer metastasis (e.g., radiation therapy, surgery and treatment using chemotherapeutic agents, such as cisplatin, carboplatin, cyclophosphamide, 5-fluorouracil, adriamycin, daunorubicin or tamoxifen).

In the present methods, the double-stranded nucleic acid molecule can be administered to the subject either as a naked double-stranded nucleic acid molecule, in conjunction with a delivery reagent, or as a recombinant plasmid or viral vector which expresses the double-stranded nucleic acid molecule.

Suitable delivery reagents for administration in conjunction with the present a double-stranded nucleic acid molecule include the Minis Transit TKO lipophilic reagent; LipoTrust™SR; lipofectin; lipofectamine; cellfectin; or polycations (e.g., polylysine); or liposomes; or collagen; atelocollagen. A preferred delivery reagent is a liposome.

Liposomes can aid in the delivery of the double-stranded nucleic acid molecule to a particular tissue, such as retinal or tumor tissue, and can also increase the blood half-life of the double-stranded nucleic acid molecule. Liposomes suitable for use in the invention are formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al., Ann Rev Biophys Bioeng 1980, 9: 467; and U.S. Pat. Nos. 4,235,871; 4,501,728; 4,837,028; and 5,019,369, the entire disclosures of which are herein incorporated by reference.

Preferably, the liposomes encapsulating the present double-stranded nucleic acid molecule comprises a ligand molecule that can deliver the liposome to the cancer site. Ligands which bind to receptors prevalent in tumor or vascular endothelial cells, such as monoclonal antibodies that bind to tumor antigens or endothelial cell surface antigens, are preferred.

Particularly preferably, the liposomes encapsulating the present double-stranded nucleic acid molecule are modified so as to avoid clearance by the mononuclear macrophage and reticuloendothelial systems, for example, by having opsonization-inhibition moieties bound to the surface of the structure. In one embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example, solid tumors, will efficiently accumulate these liposomes; see Gabizon et al., Proc Natl Acad Sci USA 1988, 18: 6949-53. In addition, the reduced uptake by the RES lowers the toxicity of stealth liposomes by preventing significant accumulation in liver and spleen. Thus, liposomes of the invention that are modified with opsonization-inhibition moieties can deliver the present double-stranded nucleic acid molecule to tumor cells.

Opsonization inhibiting moieties suitable for modifying liposomes are preferably water-soluble polymers with a molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM.sub.1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

Preferably, the opsonization-inhibiting moiety is a PEG, PPG, or derivatives thereof. Liposomes modified with PEG or PEG-derivatives are sometimes called “PEGylated liposomes”.

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a step-arylamine lipid-soluble anchor via reductive amination using Na(CN)BH. sub. 3 and a solvent mixture such as tetrahydrofuran and water in a 30:12 ratio at 60.degrees C.

Vectors expressing a double-stranded nucleic acid molecule of the invention are discussed above. Such vectors expressing at least one double-stranded nucleic acid molecule of the invention can also be administered directly or in conjunction with a suitable delivery reagent, including the Mirus Transit LT1 lipophilic reagent; LipoTrust™SR; lipofectin; lipofectamine; cellfectin; polycations (e.g., polylysine) or liposomes; or collagen; atelocollagen. Methods for delivering recombinant viral vectors, which express a double-stranded nucleic acid molecule of the invention, to an area of cancer in a patient are within the skill of the art.

The double-stranded nucleic acid molecule of the invention can be administered to the subject by any means suitable for delivering the double-stranded nucleic acid molecule into cancer sites. For example, the double-stranded nucleic acid molecule can be administered by gene gun, electroporation, or by other suitable parenteral or enteral administration routes.

Suitable enteral administration routes include oral, rectal, or intranasal delivery. Suitable parenteral administration routes include intravascular administration (e.g., intravenous bolus injection, intravenous infusion, intra-arterial bolus injection, intra-arterial infusion and catheter instillation into the vasculature); peri-tissue and intra-tissue injection (e.g., peri-tumoral and intra-tumoral injection, intra-retinal injection, or subretinal injection); subcutaneous injection or deposition including subcutaneous infusion (such as by osmotic pumps); direct application to the area at or near the site of cancer, for example by a catheter or other placement device (e.g., a retinal pellet or a suppository or an implant comprising a porous, non-porous, or gelatinous material); and inhalation. It is preferred that injections or infusions of the double-stranded nucleic acid molecule or vector be given at or near the site of cancer.

The double-stranded nucleic acid molecule of the invention can be administered in a single dose or in multiple doses. Where the administration of the double-stranded nucleic acid molecule of the invention is by infusion, the infusion can be a single sustained dose or can be delivered by multiple infusions. Injection of the agent directly into the tissue is at or near the site of cancer preferred. Multiple injections of the agent into the tissue at or near the site of cancer are particularly preferred.

One skilled in the art can also readily determine an appropriate dosage regimen for administering the double-stranded nucleic acid molecule of the invention to a given subject. For example, the double-stranded nucleic acid molecule can be administered to the subject once, for example, as a single injection or deposition at or near the cancer site. Alternatively, the double-stranded nucleic acid molecule can be administered once or twice daily to a subject for a period of from about three to about twenty-eight days, more preferably from about seven to about ten days. In a preferred dosage regimen, the double-stranded nucleic acid molecule is injected at or near the site of cancer once a day for seven days. Where a dosage regimen comprises multiple administrations, it is understood that the effective amount of a double-stranded nucleic acid molecule administered to the subject can comprise the total amount of a double-stranded nucleic acid molecule administered over the entire dosage regimen.

Compositions

Furthermore, the present invention provides pharmaceutical compositions comprising at least one of the present double-stranded nucleic acid molecules or the vectors coding for the molecules. Specifically, the present invention provides the following compositions [1] to [29]:

[1] A composition for treating cancer, comprising at least one isolated double-stranded nucleic acid molecule inhibiting the expression of a CX gene in a cell, which over-expresses the gene, wherein the CX gene is selected from the group consisting of C14orf78, MYBL2, UBE2S and UBE2T, which molecule comprises a sense strand and an antisense strand complementary thereto, hybridized to each other to form the double-stranded nucleic acid molecule and targets to a sequence selected from the group consisting of SEQ ID NOs: 47 to 57;

[2] The composition for treating cancer of [1], wherein the sense strand comprises a sequence corresponding to a target sequence selected from the group consisting of SEQ ID NOs: 47 to 57;

[3] The composition of [1], wherein the cell is a cancer cell; [4] The composition of [1], wherein the cancer to be treated is selected from the group of pancreatic cancer, lung cancer, breast cancer, bladder cancer, esophagus cancer, prostate cancer, testicular seminoma, colon cancer and cholangiocellular carcinoma;

[5] The composition of [4], wherein the lung cancer is non-small lung cancer or small lung cancer;

[6] The composition of [1], wherein the cancer to be treated is selected from the group of pancreatic cancer, cholangiocellular carcinoma or non-small cell lung cancer, when the selected CX gene is C14orf78;

[7] The composition of [1], wherein the cancer to be treated is selected from the group of pancreatic cancer, non-small lung cancer, small lung cancer, bladder cancer, esophagus cancer or testicular seminoma, when the selected CX gene is MYBL2;

[8] The composition of [1], wherein the cancer to be treated is selected from the group of pancreatic cancer, breast cancer, small lung cancer, bladder cancer, colon cancer, cholangiocellular carcinoma or prostate cancer, when the selected CX gene is UBE2S;

[9] The composition of [1], wherein the cancer to be treated is selected from the group of breast cancer, cholangiocellular carcinoma, non-small lung cancer, small lung cancer, bladder cancer, prostate cancer or esophagus cancer, when the selected CX gene is UBE2T;

[10] The composition of [1], wherein the composition contains plural kinds of the double-stranded nucleic acid molecules;

[11] The composition of [10], wherein the plural kinds of the double-stranded nucleic acid molecules target the same gene;

[12] The composition of [2], wherein the double-stranded nucleic acid molecule has a length of less than about 100 nucleotides;

[13] The composition of [12], wherein the double-stranded nucleic acid molecule has a length of less than about 75 nucleotides;

[14] The composition of [13], wherein the double-stranded nucleic acid molecule has a length of less than about 50 nucleotides;

[15] The composition of [14], wherein the double-stranded nucleic acid molecule has a length of less than about 25 nucleotides;

[16] The composition of [15], wherein the double-stranded nucleic acid molecule has a length of between about 19 and about 25 nucleotides;

[17] The composition of [2], wherein the double-stranded nucleic acid molecule consists of a single polynucleotide comprising the sense strand and the antisense strand linked by an intervening single-strand;

[18] The composition of [17], wherein the double-stranded nucleic acid molecule has the general formula 5′-[A]-[B]-[A′]-3′, wherein [A] is the sense strand sequence comprising a sequence corresponding to a target sequence selected from the group consisting of SEQ ID NOs: 47 to 57, [B] is the intervening single-strand consisting of 3 to 23 nucleotides, and [A′] is the antisense strand comprising a complementary sequence to [A];

[19] The composition of [2], wherein the double-stranded nucleic acid molecule comprises RNA;

[20] The composition of [2], wherein the double-stranded nucleic acid molecule comprises DNA and RNA;

[21] The composition of [20], wherein the double-stranded nucleic acid molecule is a hybrid of a DNA polynucleotide and an RNA polynucleotide;

[22] The composition of [21], wherein the sense and antisense strand polynucleotides consist of DNA and RNA, respectively;

[23] The composition of [20], wherein the double-stranded nucleic acid molecule is a chimera of DNA and RNA;

[24] The composition of [23], wherein at least a region flanking to the 5′-end of one or both of the sense and antisense polynucleotides consists of RNA;

[25] The composition of [24], wherein the flanking region consists of 9 to 13 nucleotides;

[26] The composition of [2], wherein the double-stranded nucleic acid molecule contains 3′ overhangs;

[27] The composition of [2], wherein the double-stranded nucleic acid molecule is encoded by a vector and contained in the composition;

[28] The composition of [27], wherein the double-stranded nucleic acid molecule has the general formula 5′-[A]-[B]-[A′]-3′, wherein [A] is the sense strand comprising a sequence corresponding to a target sequence selected from the group consisting of SEQ ID NOs: 47 to 57, [B] is a intervening single-strand consisting of 3 to 23 nucleotides, and [A′] is the antisense strand comprising a complementary sequence to [A]; and

[29] The composition of [2], wherein the composition comprises a transfection-enhancing agent and pharmaceutically acceptable carrier.

The double-stranded nucleic acid molecules of the invention are preferably formulated as pharmaceutical compositions prior to administering to a subject, according to techniques known in the art. Pharmaceutical compositions of the present invention are characterized as being at least sterile and pyrogen-free. As used herein, “pharmaceutical formulations” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is herein incorporated by reference.

The present pharmaceutical formulations comprise at least one of the double-stranded nucleic acid molecules or vectors encoding them of the present invention (e.g., 0.1 to 90% by weight), or a physiologically acceptable salt of the molecule, mixed with a physiologically acceptable carrier medium. Preferred physiologically acceptable carrier media are water, buffered water, normal saline, 0.4% saline, 0.3% glycine, hyaluronic acid and the like.

According to the present invention, the composition may contain plural kinds of the double-stranded nucleic acid molecule, each of the molecules may be directed to the same target sequence, or different target sequences within the same CX gene or on different CX genes. For example, the composition may contain double-stranded nucleic acid molecules directed to one, two, three or four of the CX genes. Alternatively, for example, the composition may contain double-stranded nucleic acid molecules directed to one, two, three, four, five or more target sequences within the same CX gene.

Furthermore, the present composition may contain a vector coding for one or plural double-stranded nucleic acid molecules. For example, the vector may encode one, two or several kinds of the present double-stranded nucleic acid molecules. Alternatively, the present composition may contain plural kinds of vectors, each of the vectors coding for a different double-stranded nucleic acid molecule.

Moreover, the present double-stranded nucleic acid molecules may be contained as liposomes in the present composition. See under the item of “Methods of treating cancer” for details of liposomes.

Pharmaceutical compositions of the invention can also comprise conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (for example calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can comprise any of the carriers and excipients listed above and 10-95%, preferably 25-75%, of one or more double-stranded nucleic acid molecule of the invention. A pharmaceutical composition for aerosol (inhalational) administration can comprise 0.01-20% by weight, preferably 1-10% by weight, of one or more double-stranded nucleic acid molecule of the invention encapsulated in a liposome as described above, and propellant. A carrier can also be included as desired; e.g., lecithin for intranasal delivery.

In addition to the above, the present composition may contain other pharmaceutical active ingredients so long as they do not inhibit the in vivo function of the present double-stranded nucleic acid molecules. For example, the composition may contain chemotherapeutic agents conventionally used for treating cancers.

In another embodiment, the present invention also provides the use of the double-stranded nucleic acid molecules of the present invention in manufacturing a pharmaceutical composition for treating a cancer expressing the CX gene. For example, the present invention relates to a use of double-stranded nucleic acid molecule inhibiting the expression of a CX gene in a cell, which over-expresses the gene, wherein the CX gene is selected from the group consisting of C14orf78, MYBL2, UBE2S and UBE2T, which molecule comprises a sense strand and an antisense strand complementary thereto, hybridized to each other to form the double-stranded nucleic acid molecule and targets to a sequence selected from the group consisting of SEQ ID NOs: 47 to 57, for manufacturing a pharmaceutical composition for treating a cancer expressing the CX gene.

Alternatively, the present invention further provides a method or process for manufacturing a pharmaceutical composition for treating a cancer expressing the CX gene, wherein the method or process comprises step for formulating a pharmaceutically or physiologically acceptable carrier with a double-stranded nucleic acid molecule inhibiting the expression of a CX gene in a cell, which over-expresses the gene, wherein the CX gene is selected from the group consisting of C14orf78, MYBL2, UBE2S and UBE2T, which molecule comprises a sense strand and an antisense strand complementary thereto, hybridized to each other to form the double-stranded nucleic acid molecule and targets to a sequence selected from the group consisting of SEQ ID NOs: 47 to 57 as active ingredients.

In another embodiment, the present invention also provides a method or process for manufacturing a pharmaceutical composition for treating a cancer expressing the CX gene, wherein the method or process comprises step for administrating an active ingredient with a pharmaceutically or physiologically acceptable carrier, wherein the active ingredient is a double-stranded nucleic acid molecule inhibiting the expression of a CX gene in a cell, which over-expresses the gene, wherein the CX gene is selected from the group consisting of C14orf78, MYBL2, UBE2S and UBE2T, which molecule comprises a sense strand and an antisense strand complementary thereto, hybridized to each other to form the double-stranded nucleic acid molecule and targets to a sequence selected from the group consisting of SEQ ID NOs: 47 to 57.

### BRIEF DESCRIPTION OF THE DRAWINGS

FIG. 1 Profiles of the four genes that were screened as candidates of therapeutic targets. Screening was performed for detecting cells expressing the target genes by RT-PCR analysis. (a):C14orf78, (b):MYBL2, (c):UBE2S and (d):UBE2T.

FIG. 2 Measurement on RNAi activity of optimized siRNA sequences against C14orf78 gene. Gene silencing activity, growth suppression effect and non-specific cell death inducing ability of siRNAs were evaluated, using cells endogenously expressing C14orf78 gene, PK-1 (a) and Panc.02.03 (b). (c) Specificity of RNAi reaction was assessed using SK-BR3 (a cell line expressing low level or no C14orf78 gene).

FIG. 3 Measurement on RNAi activity of optimized siRNA sequences against MYBL2 gene. Gene silencing activity, growth suppression effect and non-specific cell death inducing ability of siRNAs were evaluated using cells endogenously expressing MYBL2 gene, H358 (a) and TE-9 (b). (c) Specificity of RNAi reaction was assessed using SAEC (a cell line expressing low level or no MYBL2 gene).

FIG. 4 Measurement on RNAi activity of optimized siRNA sequences against UBE2S gene. Gene silencing activity, growth suppression effect and non-specific cell death inducing ability of siRNAs were evaluated using cells endogenously expressing the UBE2S gene, MCF-7 (a), PK-1 (b) and SW780 (c). (d) Specificity of RNAi reaction was assessed using HMEC (a cell line expressing low level or no UBE2S gene).

FIG. 5-1 Measurement on RNAi activity of optimized siRNA sequences against UBE2T gene. Gene silencing activity, growth suppression effect and non-specific cell death inducing ability of siRNAs were evaluated using cells endogenously expressing the UBE2T gene, MCF-7 (a), A549 (b).

FIG. 5-2 Measurement on RNAi activity of optimized siRNA sequences against UBE2T gene. Gene silencing activity, growth suppression effect and non-specific cell death inducing ability of siRNAs were evaluated using cells endogenously expressing the UBE2T gene, SW780 (c) and DU145 (d). (e) Specificity of RNAi reaction was assessed using HMEC (a cell line expressing low level or no UBE2T gene).

FIG. 6-1 In vivo antitumor activity of each siRNA against four target genes. (a) The xenograft mice were administered with LipoTrust™SR-entrapped each MYBL2 siRNA (C7, C13 and C15) or luciferase siRNA as a control by intratumoral injection. The relative tumor size at day 7 was significantly suppressed by each MYBL2 siRNA. These experiments were carried out in quintuple. The error bars represent means+/−SD. * and ** mean p<0.05 and p<0.01, respectively (Student's t-test).

FIG. 6-2 In vivo antitumor activity of each siRNA against four target genes. (b) The xenograft mice were administered with complex of atelocollagen and each siRNA against MYBL2 (C16), C14orf78 (C8, C10, C11 and C24), UBE2S (C8 and C9), UBE2T (C10) and luciferase (control) by intratumoral injection. The relative tumor size or tumor volume at day 7 was significantly suppressed by each siRNA against MYBL2, C14orf78, UBE2S and UBE2T. The error bars represent means +/−SD. * and ** mean p<0.05 and p<0.01, respectively (Student's t-test).

### EXAMPLE

The invention will be further described in the following examples, which do not limit the scope of the invention described in the claims.

### Example 1

### General Methods

Tissue Preparation

Clinical bladder cancer, cholangiocellular carcinoma, colon cancer, esophagus cancer, prostate cancer, small cell lung cancer (SCLC), pancreatic cancer, non-small cell lung cancer (NSCLC), and breast cancer samples were obtained after informed preoperative consent from 34 patients (bladder cancer), 25 patients (cholangiocellular carcinoma), 48 patients (colon cancer), 64 patients (esophagus cancer), 59 patients (prostate cancer), 15 patients (SCLC), 18 patients (pancreatic cancer), 37 patients (NSCLC), 81 patients (breast cancer) who underwent surgical resection.

cDNA Microarrays

Fabrication of the cDNA microarray slides has been described elsewhere (Zembutsu H et al., Cancer Res 2002 Jan. 15, 62(2): 518-2; Nishidate T et al., Int J Oncol 2004 October, 25(4): 797-819). For analysis of various cancer expression profiles, the present inventors prepared duplicate sets of slides containing 23,040 (colon cancer, soft tissue sarcoma, and testicular seminoma, prostate cancer) or 27,648 (breast cancer and bladder cancer) or 36,864 (pancreas cancer, NSCLC, SCLC, and esophagus cancer) cDNA spots, to reduce experimental fluctuation. Briefly, for cancer expression analysis, total RNAs were extracted from patients with tumors and from corresponding normal tissues. T7-based RNA amplification was carried out to obtain adequate quantities of RNA for microarray experiments. Aliquots of amplified RNA were labeled by reverse transcription with adequate amounts of Cy5-dCTP or Cy3-dCTP (Amersham Biosciences, Buckinghamshire, United Kingdom).

Hybridization, washing, and detection were carried out as described previously (Zembutsu H et al., Cancer Res 2002 Jan. 15, 62(2): 518-27; Nishidate T et al., Int J Oncol 2004 October, 25(4): 797-819). To detect genes that were commonly up-regulated in cancers (pancreatic cancer, NSCLC and breast cancer), overall expression patterns of all genes on the microarray were first screened to select those with expression ratios of >5.0 that were present in >20% of the cancer cases examined. Finally, to obtain therapeutic targets highly specific to target cancers, the present inventors selected genes that were not expressed in normal tissues, by reference to in-house expression database of normal human tissues.

Cell Line and Cell Culture

The present inventors prepared lung, breast, pancreatic cancer, and normal epithelial cell lines, and maintained them in adequate culture media for in vitro assay and extraction of mRNA to evaluate the target gene expression level. Lung cancer lines: A549, EBC-1, H1373, H1435, H1650, H1666, H1781, H1793, H2170, H226, H358, H520, H522, H596, PC-14, SK-LU-1, SW900, and SBC5; breast cancer lines: BT-20, BT-474, BT-549, HCC1143, HCC1500, HCC1599, HCC1937, MCF-7, MDA-MB-453, MDA-MB-453S, SK-BR-3, T47D, and ZR-75-1; pancreatic cancer lines: capan-1, capan-2, HPAF-II, KLM-1, KP-1N, MiaPaCa-2, Panc.02.03, PK-1, PK-45P, PK-59, PK-9, SUIT-2, and Panc-1; and normal epithelial lines: small airway epithelial cell (SAEC) and mammary epithelial cell (HMEC).

Semi-Quantitative RT-PCR

Selected genes were evaluated for their expression levels in normal organs (heart, liver, lung and kidney), cancer cell lines, corresponding normal tissues and normal epithelial cell lines using semi-quantitative RT-PCR experiments. Specifically, a 3-mc g aliquot of mRNA from each cell lines, normal organs and siRNA infected cells was reverse-transcribed for single-stranded cDNAs using oligo d(T)16 primer (Roche) and Superscript II (Invitrogen). Expression of alpha-actin (ACTB), beta 2 microglobulin (beta 2MG) and tubulin alpha 3 (TUBA3) served as an internal control for lung cancer, breast cancer and pancreatic cancer, respectively. Interferon induced transmembrane protein 1 (IFITM1) was used as an index of off-targeting activity of each siRNAs. PCR reactions were optimized for the number of cycles to ensure product intensity within the linear phase of amplification. Each cDNA mixture was diluted for subsequent PCR amplification with primer sets as follows:

| C14orf78: | |
|---|---|
| forward primer: | |
| 5′-GAGAAGGAAGAGGGTGAACTGAT-3′; | (SEQ ID NO: 9) |
| reverse primer: | |
| 5′-CAGTGGACATGGATAGATGAGAA-3′; | (SEQ ID NO: 10) |
| MYBL2: | |
| forward primer: | |
| 5′-GAAGCCACTTCACGACACCT-3′; | (SEQ ID NO: 11) |
| reverse primer: | |
| 5′-ATCCTAAGCAGGGTCTGAGATG-3′; | (SEQ ID NO: 12) |
| UBE2S: | |
| forward primer: | |
| 5′-TACTTCCTGACCAAGATCTTCCA-3′; | (SEQ ID NO: 13) |
| reverse primer: | |
| 5′-TTAGAGACAGAGTTGGAGGGAGG-3′; | (SEQ ID NO: 14) |
| UBE2T: | |
| forward primer: | |
| 5′-CAAATATTAGGTGGAGCCAACAC-3′; | (SEQ ID NO: 15) |
| reverse primer: | |
| 5′-TAGATCACCTTGGCAAAGAACAC-3′; | (SEQ ID NO: 16) |
| ACTB: | |
| forward primer: | |
| 5′-AGGATGCAGAAGGAGATCAC-3′; | (SEQ ID NO: 17) |
| reverse primer: | |
| 5′-AGAAAGGGTGTAACGCAACT-3′; | (SEQ ID NO: 18) |
| beta 2MG: | |
| forward primer: | |
| 5′-CACCCCCACTGAAAAAGATGA-3′; | (SEQ ID NO: 19) |
| reverse primer: | |
| 5′-TACCTGTGGAGCAACCTGC-3′; | (SEQ ID NO: 20) |
| TUBA3: | |
| forward primer: | |
| 5′-AAGGATTATGAGGAGGTTGGTGT-3′; | (SEQ ID NO: 21) |
| reverse primer: | |
| 5′-CTTGGGTCTGTAACAAAGCATTC-3′; | (SEQ ID NO: 22) |
| IFITM1: | |
| forward primer: | |
| 5′-GATCAACATCCACAGCGAGA-3′; | (SEQ ID NO: 23) |
| reverse primer: | |
| 5′-TGTCACAGAGCCGAATACCA-3′. | (SEQ ID NO: 24) |

RNAi Experiments

10 pmol/well dsRNA oligo against four candidate genes (C14orf78, MYBL2, UBE2S and UBE2T) were transfected, using Lipofectamine2000™ (Invitrogen), into cancer cells expressing the target genes and control cells on 96-well microtiter plate (Becton Dickinson). The initial concentration of cultured cells varied for each cell line. For example, PK-1 (3,000-4,000 cells/well), SK-BR-3 (4,000 cells/well), H358 (5,000-6,000 cells/well), SAEC (9,000 cells/well), MCF-7 (2,500-3,500 cells/well) and HMEC (7,000 cells/well). SiControl I (Dharmacon) was used as a negative control to avoid misinterpretation of cell death which was induced independently of siRNA specificity. SiTox (Dharmacon) was used as positive control for confirming transfection efficiency. Various sequences of gene-specific siRNAs for each candidate target sequence were tested to optimize the sequences as therapeutic drugs. After transfection, each siRNA was examined for its' growth preventing effect on cancer cells. The ability of siRNAs to knock down target genes was analyzed by RT-PCR; and the off-targeting activity of siRNAs was confirmed by monitoring up-regulation of IFITM1 which is index for interferon response elicited by common double-stranded RNA infection.

In Vivo siRNA Treatment

Screened four siRNAs (C7, C13, or C15) against MYBL2 gene were enclosed into a lipid structure of LipoTrust™SR (Hokkaido System Science) and injected intratumorally every three days into H358 xenograft mice. Briefly, 50 mc g/mL of each siRNA was mixed with 0.5 mc mol/mL of LipoTrust™SR and sonicated gently to form liposome encapsulated desired siRNA. 400 mc L of the liposome/siRNA was used for cancer treatment of mice transplanted human lung cancer cells subcutaneously. Decreased tumor development was monitored every day. Alternatively, screened siRNAs sequence against C14orf78 (C8, C10, C11 and C24); MYBL2 (C16); UBE2S (C8 and C9) and UBE2T (C10) were evaluated its therapeutic potential using atelocollagen (AteloGene™, KOKEN) as a carrier. Equal volume of AteloGene™ and 10 mc M of siRNA were mixed each other quite gently using a rotator (4 rpm) at 4 degrees C. for 20 min. Next the mixture was centrifuged (10,000 rpm) at 4 degrees C. for 1 min to defoam. 200 mc L of the mixture was injected intratumorally every three days into the tumors on shoulder of the mice. The anticancer effect of siRNAs was evaluated at 7 days after first injection in both cases.

Cell Proliferation Assay

The concentration of living cells visualized with calcein was evaluated by using IN Cell Analyzer 1000 (GE Healthcare Bio-Science KK) after 48 h, 72 h, 96 h or 120 h from transfection of siRNA.

### Example 2

### Screening of Up-Regulated Genes in Clinical Cancer Samples with No or Low Expression in Normal Organs

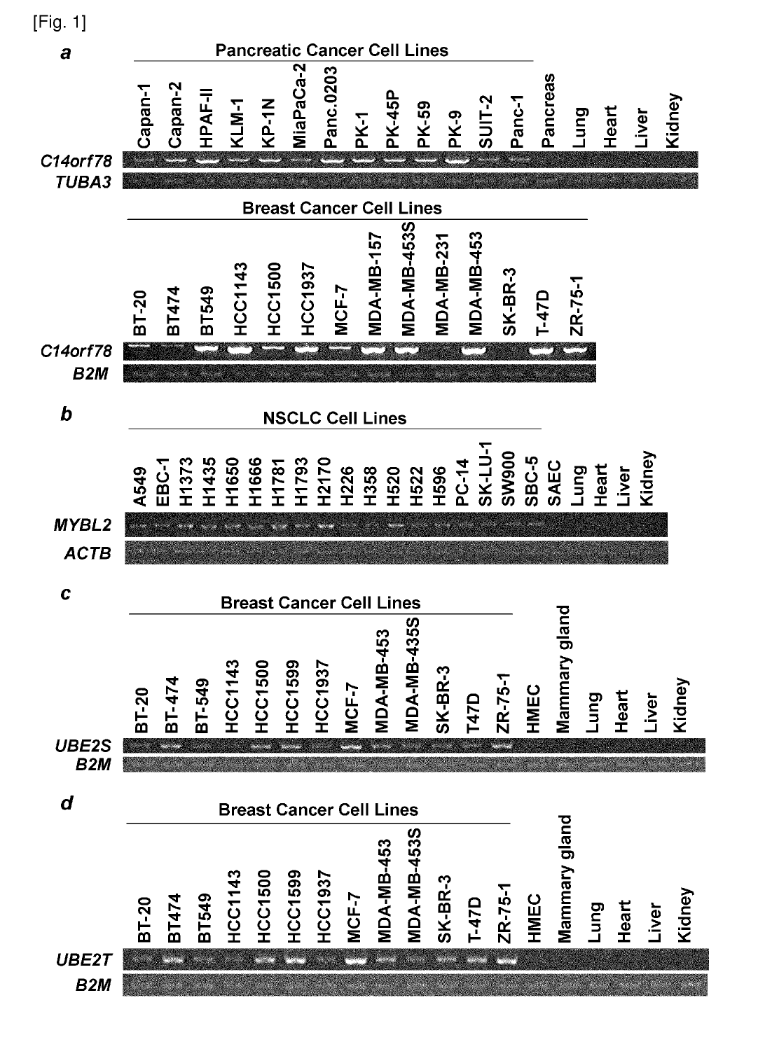

cDNA microarray analyses was carried out as described previously (Zembutsu H et al., Cancer Res 2002 Jan. 15, 62(2): 518-27; Nishidate T et al., Int J Oncol 2004 October, 25(4): 797-819). By comparing expression patterns between cancer tissues and corresponding normal epithelia, genes commonly up-regulated in the clinical cancer tissues were selected. Next, semi-quantitative RT-PCR analysis was performed to select cancer-specific genes which were detected to be highly expressed in cancer cell lines but not in corresponding normal organs and normal vital organ (FIG. 1). Genes highly expressed in normal organs were eliminated to avoid suppositious induction of fatal side effects when used as target genes to be inhibited in therapy.

### Example 3

### Design of Customized siRNA for Candidates

SiRNA sequences for each candidate genes were designed using siRNA design tool available on Ambion, Inc. website (http://www.ambion.com/techlib/misc/siRNA_finder.html) (Tuschl T et al., Genes Dev 1999 Dec. 15, 13(24): 3191-7) to select the candidate sequences of the siRNAs. Each of the siRNAs were introduced into cancer cells and control cells, and evaluated for their relative cell viability to obtain sequences that is most effective in suppressing cell growth (Table 1).

TABLE 1
Designed siRNA sequences against
the four candidate genes

| | | | | SEQ |
|---|---|---|---|---|
| Target | siRNA | | | ID |
| Gene | Name | Strand | Sequence | NO |
| C14 | C8 | target | 5′-GATATGCCATCCCAGATTT-3′ | 47 |
| orf78 | | Sense | 5′-GAUAUGCCAUCCCAGAUUUUU-3′ | 25 |
| | | Anti- | 5′-AAAUCUGGGAUGGCAUAUCUU-3′ | 26 |
| | | sense | | |
| | C10 | target | 5′-GTCAAATTCCCCAAATTAA-3′ | 48 |
| | | Sense | 5′-GUCAAAUUCCCCAAAUUAAUU-3′ | 27 |
| | | Anti- | 5′-UUAAUUUGGGGAAUUUGACUU-3′ | 28 |
| | | sense | | |
| | C11 | target | 5′-GTGTCCAGAGGCCAATATT-3′ | 49 |
| | | Sense | 5′-GUGUCCAGAGGCCAAUAUUUU-3′ | 29 |
| | | Anti- | 5′-AAUAUUGGCCUCUGGACACUU-3′ | 30 |
| | | sense | | |
| | C24 | target | 5′-GGCAGGGCTCCAAAAGACA-3′ | 50 |
| | | Sense | 5′-GGCAGGGCUCCAAAAGACAUU-3′ | 31 |
| | | Anti- | 5′-UGUCUUUUGGAGCCCUGCCUU-3′ | 32 |
| | | sense | | |
| MYBL2 | C7 | target | 5′-GGAGCCCATCGGTACAGAT-3′ | 51 |
| | | Sense | 5′-GGAGCCCAUCGGUACAGAUUU-3′ | 33 |
| | | Anti- | 5′-AUCUGUACCGAUGGGCUCCUU-3 | 34 |
| | | sense | | |
| | C13 | target | 5′-CGGCGGAGCCCCATCAAGA-3′ | 52 |
| | | Sense | 5′-CGGCGGAGCCCCAUCAAGAUU-3′ | 35 |
| | | Anti- | 5′-UCUUGAUGGGGCUCCGCCGUU-3′ | 36 |
| | | sense | | |
| | C15 | target | 5′-GCGGAGCCCCATCAAGAAA-3′ | 53 |
| | | Sense | 5′-GCGGAGCCCCAUCAAGAAAUU-3′ | 37 |
| | | Anti- | 5′-UUUCUUGAUGGGGCUCCGCUU-3′ | 38 |
| | | sense | | |
| | C16 | target | 5′-GATGTGAAGCTGATGATGT-3′ | 54 |
| | | Sense | 5′-GAUGUGAAGCUGAUGAUGUUU-3′ | 39 |
| | | Anti- | 5′-ACAUCAUCAGCUUCACAUCUU-3′ | 40 |
| | | sense | | |
| UBE2S | C8 | target | 5′-TGCTGACCATCAAGTGCCT-3′ | 55 |
| | | Sense | 5′-UGCUGACCAUCAAGUGCCUUU-3′ | 41 |
| | | Anti- | 5′-AGGCACUUGAUGGUCAGCAUU-3 | 42 |
| | | sense | | |
| | C9 | target | 5′-CCATATGCTGGAGGTCTGT-3′ | 56 |
| | | Sense | 5′-CCAUAUGCUGGAGGUCUGUUU-3′ | 43 |
| | | Anti- | 5′-ACAGACCUCCAGCAUAUGGUU-3′ | 44 |
| | | sense | | |
| UBE2T | C10 | target | 5′-AGAGAGAGCTGCACATGTT-3′ | 57 |
| | | Sense | 5′-AGAGAGAGCUGCACAUGUUUU-3′ | 45 |
| | | Anti- | 5′-AACAUGUGCAGCUCUCUCUUU-3′ | 46 |
| | | sense | | |

### Example 4

### Optimization of Gene-Specific siRNAs and Evaluation of their Silencing Specificity

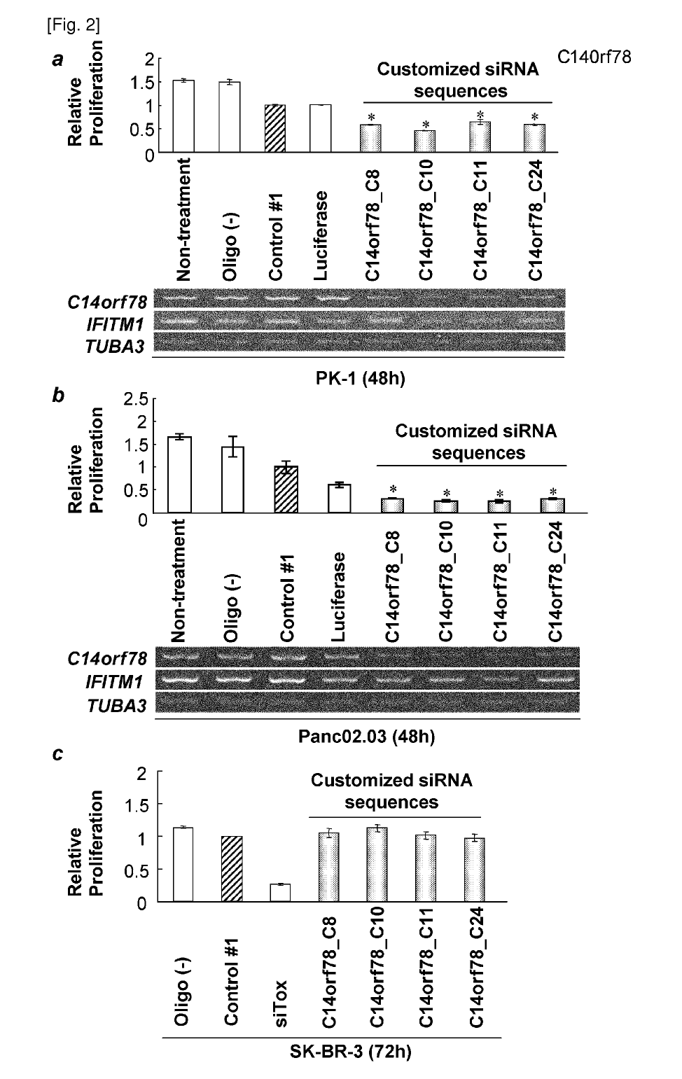

C14orf78 is a therapeutic target for pancreatic cancer because it is over-expressed (T/N ratio>=5) in clinical samples; 11 of 18 pancreatic cancers, 14 of 25 cholangiocellular carcinomas, and 10 of 37 non-small cell lung cancers (Table 2). All of the optimized siRNAs for C14orf78 (C8, C10, C11 and C24) effectively knocked down gene expression in PK-1 and Panc.02.03 coincided with suppression of cell proliferation (FIGS. 2a, b). The present inventors further examined the activation of interferon pathway by double-stranded RNA (dsRNA) against the gene. Interferon induced transmembrane protein 1 (IFITM1) is an index of interferon response resulting in undesired non-specific cell death by the infection of double-stranded RNAs. In this invention, the expression of IFITM1 was almost concordantly unchanged (FIGS. 2a, b). Furthermore, the proliferation of SK-BR-3, which is a cell line expressing low level or no C14orf78 gene, displayed no significant alteration by the infection of the siRNAs (FIG. 2c). Thus, the specificity of the present siRNAs against C14orf78 was confirmed.

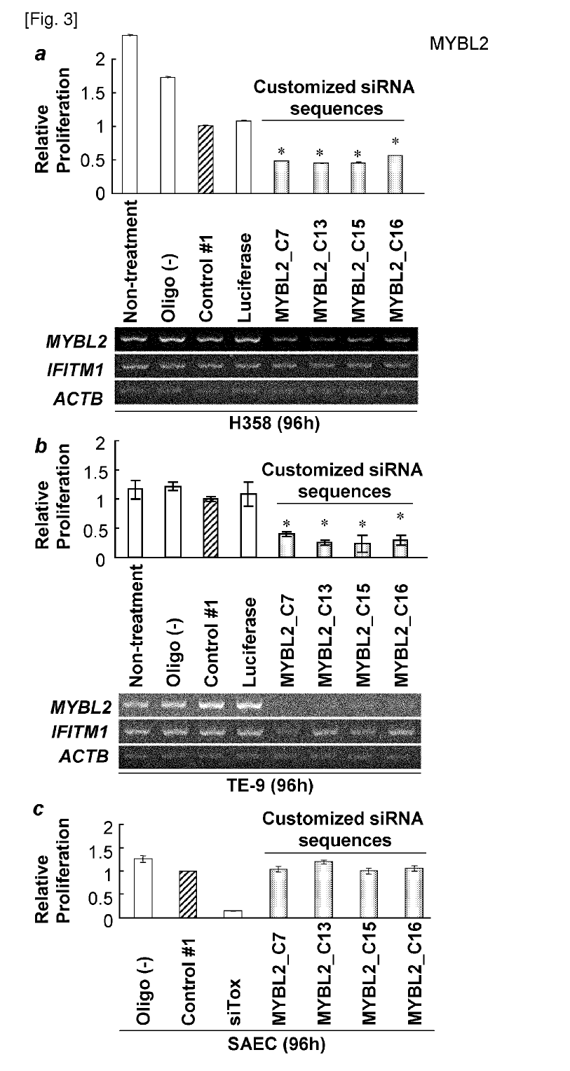

MYBL2 gene was revealed to be over-expressed in various cancers. Specifically, the gene was up-regulated (ratio>=5) in clinical samples; 18 of 34 bladder cancers, in 29 of 64 esophagus cancers, in 18 of 37 non-small cell lung cancers (NSCLC), 6 of 18 pancreatic cancers and in 14 of 15 small cell lung cancers (SCLC) (Table 2). In addition, it was reported that MYBL2 gene was also up-regulated in testicular seminoma (WO2004/031410). A recent report shows that MYBL2 protein functions as a transcription factor involved in cell cycle progression (Garcia P & Frampton J, J Cell Sci 2006 Apr. 15, 119(Pt 8): 1483-93, Epub 2006 Mar. 21). The expression profile obtained by cDNA microarray and previous reports of MYBL2 suggest that over-expression of the gene stimulates cell proliferation, and contributes to carcinogenesis or tumor development for various types of cancers. All of the screened siRNAs for MYBL2 (C7, C13, C15, and C16) significantly decreased the expression level of the gene and cell growth in NSCLC (H358) and esophagus cancer (TE-9) cell lines (FIGS. 3a, b), whereas the growth suppression induced by the siRNAs was quite stringent and limited to specific siRNAs. Actually, no activation of interferon response could be observed (FIGS. 3a, b). Moreover, no detectable growth inhibition could also be observed in normal small airway epithelial cell (SAEC), which is a MYBL2 non-expressing cell line (FIG. 3c). Thus, MYBL2 gene is an excellent target for siRNA therapy not only for NSCLC, but also SCLC, esophagus cancer, bladder cancer, testicular seminoma and pancreatic cancer. Therefore, the MYBL2-specific siRNAs of the present invention serve as powerful tools for the treatment of these cancers.

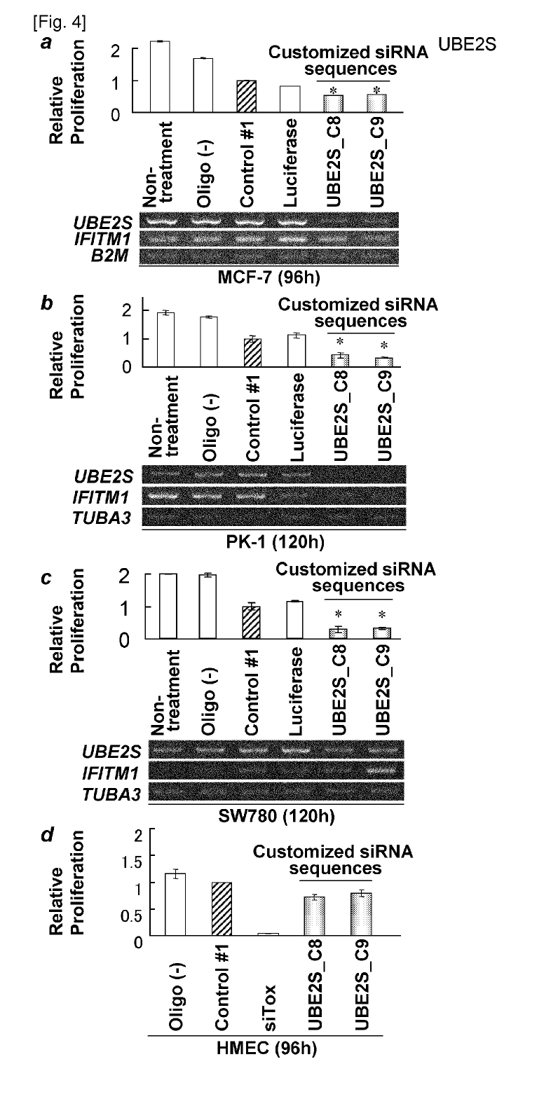

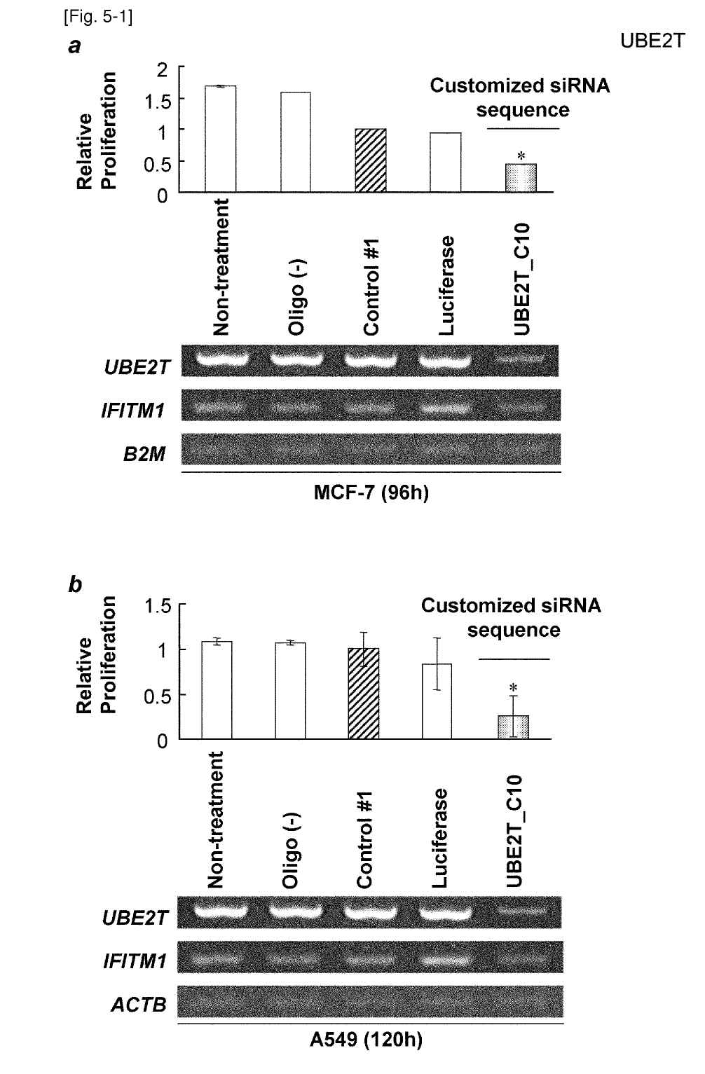

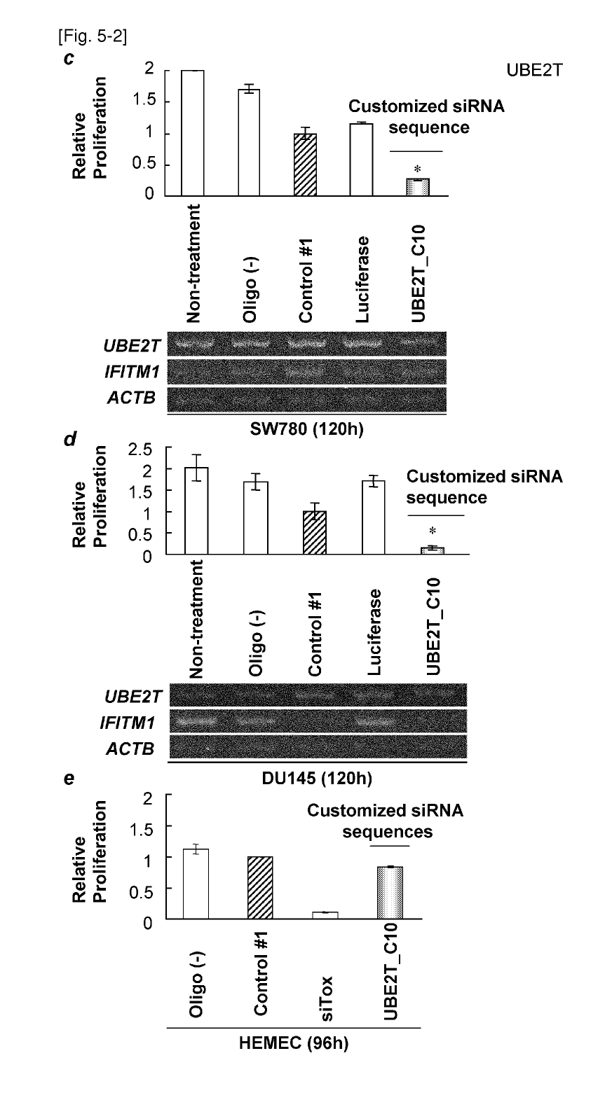

UBE2S gene was over-expressed in clinical samples; all cases of SCLCs, 29 of 34 bladder cancers, 27 of 81 breast cancers, 9 of 25 cholangiocellular carcinomas, 18 of 59 prostate cancers, 11 of 48 colon cancers, and 12 of 18 pancreatic cancers (Table 2). As is the case with the UBE2S gene encoding an ubiquitin E2 ligase like protein, UBE2T gene also showed increased expression in various type of cancers, i.e., in 12 of 25 cholangiocellular carcinoma, 12 of 15 SCLCs, in 23 of 34 bladder cancers, in 28 of 81 breast cancers, in 13 of 37 NSCLCs, 14 of 64 esophagus cancers and in 15 of 59 prostate cancers (Table 2). Selected siRNAs for UBE2S (C8 and C9) significantly decreased the expression level of the gene and cell viability in breast cancer (MCF7), pancreatic cancer (PK-1) and bladder cancer (SW780) cell lines (FIG. 4a-c). No activation of interferon response could be observed (FIG. 4a-c). Thus, undesired non-specific cell death due to double-stranded RNA infection seems not to be induced by the present siRNA. Likewise, siRNA for UBE2T (C10) effectively suppressed gene expression in breast cancer (MCF7), NSCLC (A549), bladder cancer (SW780), and prostate cancer (DU-145) (FIG. 5-1a-b, 5-2c-d). Moreover, no detectable growth inhibition could also be observed for HMEC (normal mammary epithelial cell), a cell line expressing neither UBE2S nor UBE2T (FIG. 4d, 5-2e). Accordingly, UBE2S is a therapeutic target for a wide variety of cancers including SCLC, breast, pancreas, bladder, colon, cholangiocellular and prostate cancers; UBE2T, a target for lung, bladder, breast, cholangiocellular, esophagus and prostate cancers.

TABLE 2
Over-expression (T/N ratio >=5) frequencies of screened genes in clinical cancer tissues
from cDNA microarray database

| | Bladder | Breast | Cholangiocellular | Colon | Esophagus | | Pancreatic | Prostate | |
|---|---|---|---|---|---|---|---|---|---|
| Gene | Cancer | Cancer | Cancer | Cancer | Cancer | NSCLC | Cancer | Cancer | SCLC |
| C14orf78 | 0/34 | 1/81 | 14/25 | 1/48 | 0/19 | 10/37 | 11/18 | 3/59 | 1/15 |
| MYBL2 | 18/34 | 11/81 | 5/25 | 9/48 | 29/64 | 18/37 | 6/18 | 6/59 | 14/15 |
| UBE2S | 29/34 | 27/81 | 9/25 | 11/48 | 2/64 | 1/37 | 12/18 | 18/59 | 15/15 |
| UBE2T | 23/34 | 28/81 | 12/25 | 8/48 | 14/64 | 13/37 | 3/18 | 15/59 | 12/15 |

### Example 5

### In Vivo Therapeutic Effect of Screened siRNAs Against Target Genes

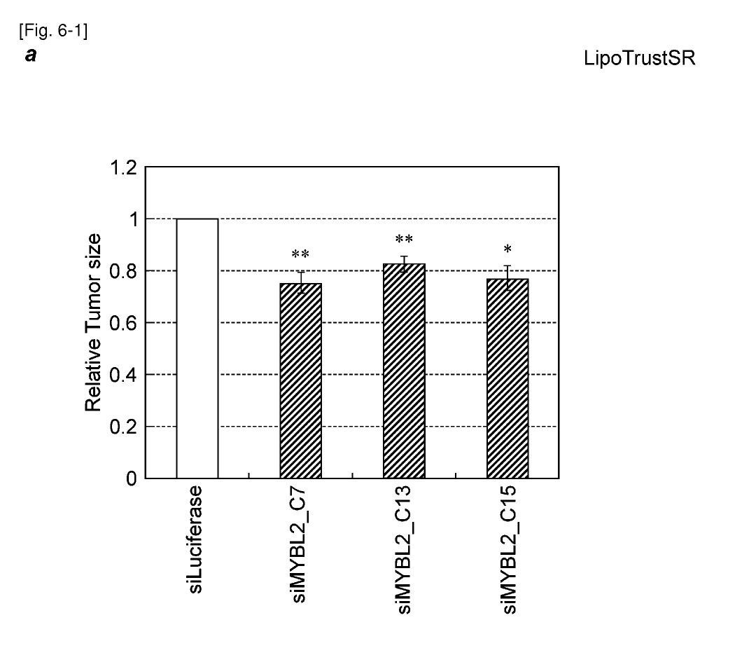

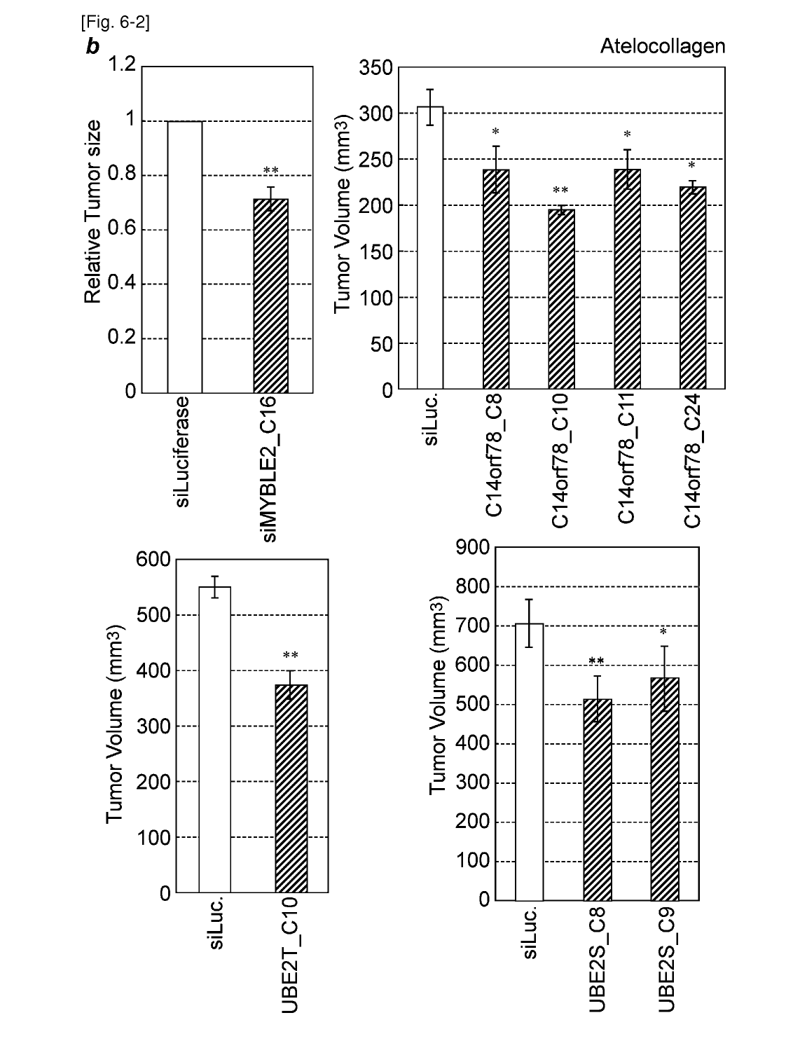

The screened siRNAs were evaluated for their therapeutic availability using in vivo model. MYBL2 siRNAs (C7, C13, C15 and C16) were enclosed into commercial liposome or atelocollagen, and injected intratumorally into nude mice transplanted H358 cells. The therapeutic efficacy by those siRNAs was evaluated by monitoring the transition of tumor size every day. The tumor size treated with LipoTrust™SR—entrapped MYBL2 siRNAs (C7, C13 and C15) at day 7 was significantly suppressed comparing with control (* p<0.05, ** p<0.01: Student's t-test)(FIG. 6-1a). On the other hand, complex of atelocollagen with siRNAs against MYBL2 (C16), C14orf78 (C8, C10, C11 and C24), UBE2S (C8 and C9) and UBE2T (C10) exerted remarkable abrogation of tumor growth compared with control siRNA when it was injected intratumorally to tumor model mice. Significant differences and +/−SD were also calculated with Student's t-test (* p<0.05; ** p<0.01) (FIG. 6-2b). Therefore screened all siRNAs against C14orf78, MYBL2, UBE2S and UBE2T could be a promising therapeutic agent for various cancers.

Discussion

In recent years, a new approach of cancer therapy using gene-specific siRNA is being used in clinical trials (Bumcrot D et al., Nat Chem Biol 2006 December, 2(12): 711-9). RNAi seems to have already earned a place among the major technology platforms (Putral LN et al., Drug News Perspect 2006 July-August, 19(6): 317-24; Frantz S, Nat Rev Drug Discov 2006 July, 5(7): 528-9; Dykxhoorn DM et al., Gene Ther 2006 March, 13(6): 541-52).

As described previously (see General Methods), the present inventors identified genes exclusively expressed in cancers and not in normal organs. In case where the double-stranded nucleic acid molecules of the present invention are used for therapy, no serious side-effects may be caused since the expression pattern of the target genes are highly specific to cancer in a quite exclusive manner. Therefore, the double-stranded nucleic acid molecules targeting cancer-specific genes of the present invention are powerful tools for the development of anticancer drugs without any adverse side-effects.

C14orf78 protein is a giant membranous protein consisting of 6,287 amino acid residues and has a PDZ domain. The PDZ domain of AHNAK1 protein, a family protein of C14orf78 protein, was bound to subunits of the L-type voltage-regulated calcium channel. Therefore, the PDZ domain of C14orf78 protein has been predicted to interact with C-terminal residues of a number of channel proteins, including those involved in calcium transport (Komuro A et al., Proc Natl Acad Sci USA 2004 Mar. 23, 101(12): 4053-8, Epub 2004 Mar. 8). Already mentioned above, AHNAK1 null mice displayed no abnormality in their phenotype and thus, AHNAK1 protein is determined not to be essential for the development or proliferation of cells. However, there is no report on the phenotype of C14orf78 knockout mice (Komuro A et al., Proc Natl Acad Sci USA 2004 Mar. 23, 101(12): 4053-8, Epub 2004 Mar. 8). Therefore, it had been unclear whether C14orf78 protein plays an important role in the development and growth of cells. In the present invention, C14orf78 protein was demonstrated as a crucial factor for cell growth or survival of pancreatic cancer cell lines. To treat malignant PDAC, the present invention provides a therapeutic agent comprising siRNAs which target C14orf78 gene.

Among a number of over-expressed genes identified by genome-wide cDNA microarray (Kikuchi T et al., Oncogene 2003 Apr. 10, 22(14): 2192-205), MYBL2 gene was selected for further detailed analysis due to obvious signal intensity in cancer cells detected by cDNA microarray (more than 5 times compared to that in normal lung). Restricted expression in normal adult tissue is an important factor for a molecule to be used as a target of siRNA for treating cancer, considering the side effect of the treatment. Furthermore, in-house database of gene expression profile of various clinical cancers revealed significant over-expression of MYBL2 gene (ratio>=5) in bladder cancers, esophagus cancers, NSCLC, SCLC, pancreatic cancer (see Result), and soft tissue sarcomas (data not shown) and testicular tumors as described (see Results). Previous study of MYBL2 null (−/−) mice proved MYBL2 protein essential for embryonic development; the mice being dead at about E4.5 (Tanaka Y et al., J Biol Chem 1999 Oct. 1, 274(40): 28067-70). Almost no MYBL2 gene expression was detected in normal adult tissues, whereas abundant expression was detected in embryonic tissues and cancers. Therefore, MYBL2 gene might be involved in carcinogenesis and tumor development, and may serve as an excellent molecular target for treating a wide variety of cancers with low risk of adverse side-effects.

SMART program (http://smart.embl-heidelberg.de/) predicted that both UBE2T and UBE2S proteins contain an UBCc domain (Ubiquitin-conjugating enzyme E2, catalytic domain homologues), suggesting the two proteins to have a potential E2 ubiquitin enzyme activity via mono-ubiquitination and being involved in tumorigenesis of breast cancer. Many previous studies reported that deregulation of E3 ligase results in cancer development (Yen L et al., Cancer Res 2006 December 1, 66(23): 11279-86; Ohh M, Neoplasia 2006 August, 8(8): 623-9; Lisztwan J et al., Genes Dev 1999 Jul. 15, 13(14): 1822-33), only a few reports indicated that E2 ligase might be involved in cancer development (Jung C R et al., Nat Med 2006 July, 12(7): 809-16, Epub 2006 Jul. 2; Okamoto Y et al., Cancer Res 2003 Jul. 15, 63(14): 4167-73). Previous study reported that UBE2 family proteins (UBE2s) are putative ubiquitin-conjugating enzymes (E2 ligase) which contribute to the proteolytic pathway. However, details of the function of UBE2s in cancers are still unknown and research revealing whether they only have an E2 ligase activity in the proteolytic pathway or have other in vivo properties is being awaited.

### INDUSTRIAL APPLICABILITY

The present inventors have shown that the cell growth is suppressed by double-stranded nucleic acid molecules that specifically target the C14orf78, MYBL2, UBE2S and UBE2T gene. Thus, these novel double-stranded nucleic acid molecules are useful candidates for the development of anti-cancer pharmaceuticals. For example, agents that block the expression of C14orf78, MYBL2, UBE2S or UBE2T protein or prevent its activity may find therapeutic utility as anti-cancer agents, particularly anti-cancer agents for the treatment of lung cancers, breast cancers, bladder cancers, cholangiocellular carcinoma, esophagus cancers, prostate cancer, prostate cancer or testicular seminomas.

While the invention has been described in detail and with reference to specific embodiments thereof, it will be apparent to one skilled in the art that various changes and modifications can be made therein without departing from the spirit and scope of the invention. All publications, patent applications, patents, and other references mentioned herein are incorporated by reference in their entirety. In case of conflict, the present specification, including definitions, will control. In addition, the materials, methods, and examples are illustrative only and not intended to be limiting.

1. An isolated double-stranded nucleic acid molecule, which, when introduced into a cell, inhibits expression of a CX gene and cell growth of cells expressing the CX-gene, wherein the CX gene is selected from the group consisting of C14orf78, MYBL2, UBE2S and UBE2T, which double-stranded nucleic acid molecule targets a sequence selected from the group consisting of SEQ ID NOs: 47 to 57.

2. The isolated double-stranded nucleic acid molecule of claim 1, which has a sense strand which comprises a sequence corresponding to a target sequence selected from the group consisting of SEQ ID NOs: 47 to 57.

3. The double-stranded nucleic acid molecule of claim 2, which has a length of between about 19 and about 25 nucleotides.

4. The double-stranded nucleic acid molecule of claim 1, which consists of a single polynucleotide comprising both a sense strand and an antisense strand linked by an intervening single-strand.

5. The double-stranded nucleic acid molecule of claim 4, which has the general formula 5′-[A]-[B]-[A′]-3′, wherein [A] is the sense strand comprising a sequence corresponding to a target sequence selected from the group consisting of SEQ ID NOs: 47 to 57, [B] is the intervening single-strand consisting of 3 to 23 nucleotides, and [A′] is the antisense strand comprising a complementary sequence to [A].

6. The double-stranded nucleic acid molecule of claim 1, which contains a 3′ overhang.

7. A vector expressing the double-stranded nucleic acid molecule of claim 1.

8. The vector of claim 7, wherein the double-stranded nucleic acid molecule has the general formula 5′-[A]-[B]-[A′]-3′, wherein [A] is a sense strand comprising a sequence corresponding to a target sequence selected from the group consisting of SEQ ID NO: 47 to SEQ ID NO: 57, [B] is an intervening single-strand consisting of 3 to 23 nucleotides, and [A′] is an antisense strand comprising a complementary sequence to [A].

9. A method for treating cancer comprising the step of administering at least one isolated double-stranded nucleic acid molecule which inhibits the expression of a CX gene in a cell, which over-expresses the gene, wherein the CX gene is selected from the group consisting of C14orf78, MYBL2, UBE2S and UBE2T, which double-stranded nucleic acid molecule targets a sequence selected from the group consisting of SEQ ID NOs: 47 to 57.

10. The method of claim 9, wherein the sense strand comprises a sequence corresponding to a target sequence selected from the group consisting of SEQ ID NOs: 47 to 57.

11. The method of claim 9, wherein the cancer to be treated is selected from the group consisting of:
(i) pancreatic cancer, cholangiocellular carcinoma and non-small cell lung cancer, when the selected CX gene is C14orf78;
(ii) pancreatic cancer, non-small cell lung cancer, small cell lung cancer, bladder cancer, esophagus cancer and testicular seminoma, when the selected CX gene is MYBL2;
(iii) pancreatic cancer, breast cancer, prostate cancer, small cell lung cancer, bladder cancer, cholangiocellular carcinoma and colon cancer, when the selected CX gene is UBE2S; and
(iv) breast cancer, non-small cell lung cancer, small cell lung cancer, bladder cancer, cholangiocellular carcinoma, prostate cancer and esophagus cancer, when the selected CX gene is UBE2T.

12. The method of claim 9, wherein more than one of the double-stranded nucleic acid molecules are administered.

13. The method of claim 10, wherein the double-stranded nucleic acid molecule has a length of between about 19 and about 25 nucleotides in length.

14. The method of claim 9, wherein the double-stranded nucleic acid molecule consists of a single polynucleotide comprising a sense strand and a antisense strand linked by an intervening single-strand.

15. The method of claim 14, wherein the double-stranded nucleic acid molecule has the general formula 5′-[A]-[B]-[A′]-3′, wherein [A] is the sense strand comprising a sequence corresponding to a target sequence selected from the group consisting of SEQ ID NOs: 47 to 57, [B] is the intervening single strand consisting of 3 to 23 nucleotides, and [A′] is the antisense strand comprising a complementary sequence to [A].

16. The method of claim 9, wherein the double-stranded nucleic acid molecule contains 3′ overhangs.

17. The method of claim 9, wherein the double-stranded nucleic acid molecule is encoded by a vector.

18. The method of claim 17, wherein the double-stranded nucleic acid molecule encoded by the vector has the general formula 5′-[A]-[B]-[A′]-3′, wherein [A] is a sense strand comprising a sequence corresponding to a target sequence selected from the group consisting of SEQ ID NOs: 47 to 57, [B] is an intervening single strand consisting of 3 to 23 nucleotides, and [A′] is a antisense strand comprising a complementary sequence to [A].

19. The method of claim 9, wherein the double-stranded nucleic acid molecule is contained in a composition which comprises in addition to the molecule a transfection-enhancing agent and pharmaceutically acceptable carrier.

20. A composition for treating cancer, comprising at least one isolated double-stranded nucleic acid molecule, which inhibits the expression of a CX gene in a cell, which over-expresses the gene, wherein the CX gene is selected from the group consisting of C14orf78, MYBL2, UBE2S and UBE2T, which double-stranded nucleic acid molecule targets a sequence selected from the group consisting of SEQ ID NOs: 47 to 57.

21. The composition of claim 20, wherein the double-stranded nucleic acid molecule has a sense strand which comprises a sequence corresponding to a target sequence selected from the group consisting of SEQ ID NOs: 47 to 57.

22. The composition of claim 20, wherein the cancer to be treated is selected from the group consisting of:
(i) pancreatic cancer, cholangiocellular carcinoma and non-small cell lung cancer, when the selected CX gene is C14orf78;
(ii) pancreatic cancer, non-small cell lung cancer, small cell lung cancer, bladder cancer, esophagus cancer and testicular seminoma, the selected CX gene is MYBL2;
(iii) pancreatic cancer, breast cancer, cholangiocellular carcinoma, prostate cancer, small cell lung cancer, bladder cancer and colon cancer, when the selected CX gene is UBE2S; and
(iv) breast cancer, non-small cell lung cancer, small cell lung cancer, bladder cancer, cholangiocellular carcinoma, prostate cancer and esophagus cancer, when the selected CX gene is UBE2T.

23. The composition of claim 20, wherein the composition contains more than one of the double-stranded nucleic acid molecules.

24. The composition of claim 21, wherein the double-stranded nucleic acid molecule has a length of between about 19 and about 25 nucleotides.

25. The composition of claim 20, wherein the double-stranded nucleic acid molecule consists of a single polynucleotide comprising a sense strand and an antisense strand linked by an intervening single-strand.

26. The composition of claim 25, wherein the double-stranded nucleic acid molecule has the general formula 5′-[A]-[B]-[A′]-3′, wherein [A] is the sense strand sequence comprising a sequence corresponding to a target sequence selected from the group consisting of SEQ ID NOs: 47 to 57, [B] is the intervening single-strand consisting of 3 to 23 nucleotides, and [A] is the antisense strand comprising a complementary sequence to [A].

27. The composition of claim 20, wherein the double-stranded nucleic acid molecule contains a 3′ overhang.

28. The composition of claim 20, wherein the double-stranded nucleic acid molecule is encoded by a vector and contained in the composition.

29. The composition of claim 28, wherein the double-stranded nucleic acid molecule has the general formula 5′-[A]-[B]-[A′]-3′, wherein [A] is a sense strand comprising a sequence corresponding to a target sequence selected from the group consisting of SEQ ID NOs: 47 to 57, [B] is an intervening single-strand consisting of 3 to 23 nucleotides, and [A′] is an antisense strand comprising a complementary sequence to [A].

30. The composition of claim 20, wherein the composition comprises a transfection enhancing agent and pharmaceutically acceptable carrier.
